(19) United States
(12) Patent Application Publication
(10) Pub. No.: US 2015/0240264 A1
(43) Pub. Date: Aug. 27, 2015

(54) METHODS FOR CO-PRODUCTION OF A TERPENE, SUCCINATE AND HYDROGEN

(71) Applicant: BRASKEM S.A., CAMACARI (BR)

(72) Inventors: Mateus Schreiner Garcez Lopes, Camacari (BR); Debora Noma Okamoto, Camacari (BR); Iuri Estrada Gouvea, Camacari (BR)

(21) Appl. No.: 14/627,205

(22) Filed: Feb. 20, 2015

Related U.S. Application Data
(60) Provisional application No. 61/942,933, filed on Feb. 21, 2014.

(51) Int. Cl. C12P 5/00; C12P 3/00; C12N 9/90; C12N 9/04; C12N 9/02; C12N 9/10; C12P 7/46; C12N 9/88

(52) U.S. Cl. CPC C12P 5/007; C12P 7/46; C12P 3/00; C12N 9/88; C12N 9/0006; C12N 9/001; C12N 9/1029; C12N 9/0008; C12N 9/0067; C12N 9/1025; C12N 9/90; C12N 9/1085

(57) ABSTRACT

The present disclosure generally relates to microorganisms (e.g., non-naturally occurring microorganisms) that comprise one or more polynucleotides coding for enzymes in a pathway that catalyze a conversion of a carbon source (e.g., a fermentable carbon source) to terpene, succinate, and hydrogen and the use of such microorganisms for the production of terpene, succinate, and hydrogen.

### FIELD

The present disclosure generally relates to microorganisms (e.g., non-naturally occurring microorganisms) that comprise one or more polynucleotides coding for enzymes in a pathway that catalyze the conversion of a carbon source to one or more terpenes, succinate and hydrogen.

### BACKGROUND

For many years, the chemical industry has been using coal, gas, and oil to produce the vast majority of its industrial products. However, with diminishing supplies of these resources and the looming dangers of excessive carbon dioxide emissions, there is a dire need to develop a sustainable and renewable chemical that can produce the same products in a safe and cost effective way.

Terpenes are derived biosynthetically from units of isoprene, which has the molecular formula C₅H₈. The basic molecular formulae of terpenes are multiples of that, (C₅H₈)ₙ where n is the number of linked isoprene units. The isoprene units may be linked together “head to tail” to form linear chains or they may be arranged to form rings. One can consider the isoprene unit as one of nature's common building blocks. Isoprene itself does not undergo the building process, but rather activated forms, isopentenyl pyrophosphate (IPP or also isopentenyl diphosphate) and dimethylallyl pyrophosphate (DMAPP or also dimethylallyl diphosphate), are the components in the biosynthetic pathway. IPP is isomerized to DMAPP by the enzyme isopentenyl pyrophosphate isomerase. As chains of isoprene units are built up, the resulting terpenes are classified sequentially by size as hemiterpenes, monoterpenes, sesquiterpenes, diterpenes, sesterterpenes, triterpenes, and tetraterpenes. Essentially, they are all synthesized by terpene synthase. Terpenes may be classified by the number of isoprene units in the molecule; a prefix in the name indicates the number of terpene units needed to assemble the molecule.

Given the world-wide demand for terpenes, more economical methods for producing terpenes are needed. In particular, methods that produce terpenes at rates, titers, and purity that are sufficient to meet the demands of a robust commercial process are desirable. Also desired are systems for producing terpenes from inexpensive starting materials without current production drawbacks including the use of toxic and/or expensive catalysts, and highly flammable and/or gaseous carbon sources.

### SUMMARY

The present disclosure generally relates to microorganisms (e.g., non-naturally occurring microorganisms) that comprise one or more polynucleotides coding for enzymes in a pathway that catalyze the conversion of a carbon source to one or more terpenes, having the formula (C₅H₈)ₙ such as isoprene and succinate. In an embodiment the pathways that catalyze the production of a terpene and succinate in a microorganism occur under anaerobic conditions.

The present disclosure also provides a method of co-producing farnesene, succinate, and hydrogen from a glucose source (e.g., sugarcane juice, sugarcane molasses, hydrolyzed starch, hydrolyzed lignocellulosic materials, glucose, sucrose, fructose, lactate, lactose, xylose, pyruvate, or glycerol in any form, or any combination thereof) comprising: providing a glucose source; expressing one or more exogenous polynucleotides in a microorganism that encode one or more enzymes in a pathway that catalyze a conversion of glucose to phosphoenolpyruvate, one or more polynucleotides coding for enzymes in a pathway that catalyze a conversion of phosphoenolpyruvate to oxaloacetate, one or more polynucleotides coding for enzymes in a pathway that catalyze a conversion of oxaloacetate to malate, one or more polynucleotides coding for enzymes in a pathway that catalyze a conversion of malate to fumarate, one or more polynucleotides coding for enzymes in a pathway that catalyze a conversion of fumarate to succinate, one or more polynucleotides coding for enzymes in a pathway that catalyze a conversion of pyruvate to acetyl-CoA and formate, one or more polynucleotides coding for enzymes in a pathway that catalyze a conversion of formate to CO₂ or formate to CO₂ and hydrogen, one or more polynucleotides coding for enzymes in a pathway that catalyze a conversion of NAD(P)H to NAD(P)⁺ and hydrogen, or a conversion of a reduced acceptor to CO₂ and hydrogen, one or more polynucleotides coding for enzymes in a pathway that catalyze a conversion of acetoacetyl-CoA and acetyl-CoA to 3-hydroxy-3-methylglutaryl-CoA, one or more polynucleotides coding for enzymes in a pathway that catalyze a conversion of 3-hydroxy-3-methylglutaryl-CoA to mevalonate, one or more polynucleotides coding for enzymes in a pathway that catalyze a conversion of mevalonate to phosphomevalonate, one or more polynucleotides coding for enzymes in a pathway that catalyze a conversion of phosphomevalonate to diphosphomevalonate, one or more polynucleotides coding for enzymes in a pathway that catalyze a conversion of diphosphomevalonate to isopentenyl diphosphate, one or more polynucleotides coding for enzymes in a pathway that catalyze a conversion of isopentenyl diphosphate to dimethylallyl diphosphate, one or more polynucleotides coding for enzymes in a pathway that catalyze a conversion of dimethylallyl diphosphate to isoprene, one or more polynucleotides coding for enzymes in a pathway that catalyze a conversion of dimethylallyl diphosphate and isopentenyl diphosphate to diphosphate and geranyl diphosphate, one or more polynucleotides coding for enzymes in a pathway that catalyze a conversion of geranyl diphosphate and isopentenyl diphosphate to diphosphate and farnesyl diphosphate, and/or one or more polynucleotides coding for enzymes in a pathway that catalyze a conversion of farnesyl diphosphate to farnesene; and contacting the glucose source with the microorganism, wherein the co-production method is anaerobic.

In some embodiments of each or any of the above or below mentioned embodiments, the enzymes in the pathway that catalyze a conversion of phosphoenolpyruvate to oxaloacetate include a PEP carboxykinase, wherein the enzymes in the pathway that catalyze a conversion of oxaloacetate to malate include a malate dehydrogenase, wherein the enzymes in the pathway that catalyze a conversion of malate to fumarate include a fumarate hydratase; wherein the enzymes in the pathway that catalyze a conversion of fumarate and a reduced acceptor to succinate and an acceptor include a fumarate dehydrogenase or a succinate dehydrogenase, wherein the enzymes in the pathway that catalyze a conversion of pyruvate to acetyl-CoA and formate include a pyruvate formate lyase; wherein the enzymes in the pathway that catalyze a conversion of formate to CO₂, or formate to CO₂ and H₂ include a formate dehydrogenase or a formate hydrogen lyase; wherein the enzymes in the pathway that catalyze a conversion of NAD(P)H to NAD(P)⁺ and hydrogen, or a conversion of a reduced acceptor to CO₂ and hydrogen include a hydrogen dehydrogenase or a hydrogen dehydrogenase (NADP+); wherein the enzymes in the pathway that catalyze a conversion of acetoacetyl-CoA and acetyl-CoA to 3-hydroxy-3-methylglutaryl-CoA include a hydroxymethylglutaryl-CoA synthase, wherein the one or more polynucleotides coding for enzymes in the pathway that catalyze a conversion of 3-hydroxy-3-methylglutaryl-CoA to mevalonate include a hydroxymethylglutaryl-CoA reductase; wherein the enzymes in the pathway that catalyze a conversion of mevalonate to phosphomevalonate include a mevalonate kinase; wherein the enzymes in the pathway that catalyze a conversion of phosphomevalonate to diphosphomevalonate include a phosphomevalonate kinase; wherein the enzymes in the pathway that catalyze a conversion of diphosphomevalonate to isopentenyl diphosphate include a diphosphomevalonate decarboxylase; wherein the enzymes in the pathway that catalyze a conversion of isopentenyl diphosphate to dimethylallyl diphosphate include an isopentenyl diphosphate delta-isomerase; wherein the enzymes in the pathway that catalyze a conversion of dimethylallyl diphosphate to isoprene include an isoprene synthase; wherein the enzymes in the pathway that catalyze a conversion of dimethylallyl diphosphate and isopentenyl diphosphate to diphosphate and geranyl diphosphate include a geranyl-diphosphate synthase; wherein the enzymes in the pathway that catalyze a conversion of geranyl diphosphate and isopentenyl diphosphate to diphosphate and farnesyl diphosphate include a farnesyl pyrophosphate synthase; and wherein the enzymes in the pathway that catalyze a conversion of farnesyl diphosphate to farnesene include a farnesene synthase.

In some embodiments of each or any of the above or below mentioned embodiments, the microorganism is a bacteria selected from the genera consisting essentially of: Propionibacterium, Propionispira, Clostridium, Bacillus, Escherichia, Pelobacter, or Lactobacillus.

In some embodiments of each or any of the above or below mentioned embodiments, the microorganism is a eukaryote selected from the group consisting essentially of a yeast, filamentous fungi, protozoa, or algae.

In some embodiments of each or any of the above or below mentioned embodiments, the microorganism is from a genus selected from the group consisting of: Saccharomyces, Yarrowia, Hansenula, Pichia, Ashbya, and Candida.

In some embodiments of each or any of the above or below mentioned embodiments, farnesene, succinate, and hydrogen are secreted by the microorganism into the fermentation media.

In some embodiments of each or any of the above or below mentioned embodiments, the methods further comprise recovering farnesene, succinate, and hydrogen from the fermentation media.

In some embodiments of each or any of the above or below mentioned embodiments, the glucose source (e.g., sugarcane juice, sugarcane molasses, hydrolyzed starch, hydrolyzed lignocellulosic materials, glucose, sucrose, fructose, lactate, lactose, xylose, pyruvate, or glycerol in any form, or any combination thereof) is contacted with the microorganism prior to expressing in the microorganism the one or more exogenous polynucleotides in a microorganism that encode one or more enzymes in a pathway that catalyze a conversion of glucose to phosphoenolpyruvate, one or more polynucleotides coding for enzymes in a pathway that catalyze a conversion of phosphoenolpyruvate to oxaloacetate, one or more polynucleotides coding for enzymes in a pathway that catalyze a conversion of oxaloacetate to malate, one or more polynucleotides coding for enzymes in a pathway that catalyze a conversion of malate to fumarate, one or more polynucleotides coding for enzymes in a pathway that catalyze a conversion of fumarate to succinate, one or more polynucleotides coding for enzymes in a pathway that catalyze a conversion of pyruvate to acetyl-CoA and formate, one or more polynucleotides coding for enzymes in a pathway that catalyze a conversion of formate to CO₂ or formate to CO₂ and hydrogen, one or more polynucleotides coding for enzymes in a pathway that catalyze a conversion of NAD(P)H to NAD(P)⁺ and hydrogen, or a conversion of a reduced acceptor to CO₂ and hydrogen, one or more polynucleotides coding for enzymes in a pathway that catalyze a conversion of acetoacetyl-CoA and acetyl-CoA to 3-hydroxy-3-methylglutaryl-CoA, one or more polynucleotides coding for enzymes in a pathway that catalyze a conversion of 3-hydroxy-3-methylglutaryl-CoA to mevalonate, one or more polynucleotides coding for enzymes in a pathway that catalyze a conversion of mevalonate to phosphomevalonate, one or more polynucleotides coding for enzymes in a pathway that catalyze a conversion of phosphomevalonate to diphosphomevalonate, one or more polynucleotides coding for enzymes in a pathway that catalyze a conversion of diphosphomevalonate to isopentenyl diphosphate, one or more polynucleotides coding for enzymes in a pathway that catalyze a conversion of isopentenyl diphosphate to dimethylallyl diphosphate, one or more polynucleotides coding for enzymes in a pathway that catalyze a conversion of dimethylallyl diphosphate to isoprene, one or more polynucleotides coding for enzymes in a pathway that catalyze a conversion of dimethylallyl diphosphate and isopentenyl diphosphate to diphosphate and geranyl diphosphate, one or more polynucleotides coding for enzymes in a pathway that catalyze a conversion of geranyl diphosphate and isopentenyl diphosphate to diphosphate and farnesyl diphosphate, and one or more polynucleotides coding for enzymes in a pathway that catalyze a conversion of farnesyl diphosphate to farnesene.

In some embodiments of each or any of the above or below mentioned embodiments, the glucose source (e.g., sugarcane juice, sugarcane molasses, hydrolyzed starch, hydrolyzed lignocellulosic materials, glucose, sucrose, fructose, lactate, lactose, xylose, pyruvate, or glycerol in any form, or any combination thereof) is contacted with the microorganism after expressing in the microorganism the one or more exogenous polynucleotides in a microorganism that encode one or more enzymes in a pathway that catalyze a conversion of glucose to phosphoenolpyruvate, one or more polynucleotides coding for enzymes in a pathway that catalyze a conversion of phosphoenolpyruvate to oxaloacetate, one or more polynucleotides coding for enzymes in a pathway that catalyze a conversion of oxaloacetate to malate, one or more polynucleotides coding for enzymes in a pathway that catalyze a conversion of malate to fumarate, one or more polynucleotides coding for enzymes in a pathway that catalyze a conversion of fumarate to succinate, one or more polynucleotides coding for enzymes in a pathway that catalyze a conversion of pyruvate to acetyl-CoA and formate, one or more polynucleotides coding for enzymes in a pathway that catalyze a conversion of formate to CO₂ or formate to CO₂ and hydrogen, one or more polynucleotides coding for enzymes in a pathway that catalyze a conversion of NAD(P)H to NAD(P)⁺ and hydrogen, or a conversion of a reduced acceptor to CO₂ and hydrogen, one or more polynucleotides coding for enzymes in a pathway that catalyze a conversion of acetoacetyl-CoA and acetyl-CoA to 3-hydroxy-3-methylglutaryl-CoA, one or more polynucleotides coding for enzymes in a pathway that catalyze a conversion of 3-hydroxy-3-methylglutaryl-CoA to mevalonate, one or more polynucleotides coding for enzymes in a pathway that catalyze a conversion of mevalonate to phosphomevalonate, one or more polynucleotides coding for enzymes in a pathway that catalyze a conversion of phosphomevalonate to diphosphomevalonate, one or more polynucleotides coding for enzymes in a pathway that catalyze a conversion of diphosphomevalonate to isopentenyl diphosphate, one or more polynucleotides coding for enzymes in a pathway that catalyze a conversion of isopentenyl diphosphate to dimethylallyl diphosphate, one or more polynucleotides coding for enzymes in a pathway that catalyze a conversion of dimethylallyl diphosphate to isoprene, one or more polynucleotides coding for enzymes in a pathway that catalyze a conversion of dimethylallyl diphosphate and isopentenyl diphosphate to diphosphate and geranyl diphosphate, one or more polynucleotides coding for enzymes in a pathway that catalyze a conversion of geranyl diphosphate and isopentenyl diphosphate to diphosphate and farnesyl diphosphate, and one or more polynucleotides coding for enzymes in a pathway that catalyze a conversion of farnesyl diphosphate to farnesene.

The present disclosure also provides a microorganism comprising one or more polynucleotides coding for enzymes in a pathway that catalyze a conversion of phosphoenolpyruvate to oxaloacetate, one or more polynucleotides coding for enzymes in a pathway that catalyze a conversion of oxaloacetate to malate, one or more polynucleotides coding for enzymes in a pathway that catalyze a conversion of malate to fumarate, one or more polynucleotides coding for enzymes in a pathway that catalyze a conversion of fumarate to succinate, one or more polynucleotides coding for enzymes in a pathway that catalyze a conversion of pyruvate to acetyl-CoA and formate, one or more polynucleotides coding for enzymes in a pathway that catalyze a conversion of formate to CO₂ or formate to CO₂ and hydrogen, one or more polynucleotides coding for enzymes in a pathway that catalyze a conversion of NAD(P)H to NAD(P)⁺ and hydrogen, or a conversion of a reduced acceptor to CO₂ and hydrogen, one or more polynucleotides coding for enzymes in a pathway that catalyze a conversion of acetoacetyl-CoA and acetyl-CoA to 3-hydroxy-3-methylglutaryl-CoA, one or more polynucleotides coding for enzymes in a pathway that catalyze a conversion of 3-hydroxy-3-methylglutaryl-CoA to mevalonate, one or more polynucleotides coding for enzymes in a pathway that catalyze a conversion of mevalonate to phosphomevalonate, one or more polynucleotides coding for enzymes in a pathway that catalyze a conversion of phosphomevalonate to diphosphomevalonate, one or more polynucleotides coding for enzymes in a pathway that catalyze a conversion of diphosphomevalonate to isopentenyl diphosphate, one or more polynucleotides coding for enzymes in a pathway that catalyze a conversion of isopentenyl diphosphate to dimethylallyl diphosphate, one or more polynucleotides coding for enzymes in a pathway that catalyze a conversion of dimethylallyl diphosphate to isoprene, one or more polynucleotides coding for enzymes in a pathway that catalyze a conversion of dimethylallyl diphosphate and isopentenyl diphosphate to diphosphate and geranyl diphosphate, one or more polynucleotides coding for enzymes in a pathway that catalyze a conversion of geranyl diphosphate and isopentenyl diphosphate to diphosphate and farnesyl diphosphate, and/or one or more polynucleotides coding for enzymes in a pathway that catalyze a conversion of farnesyl diphosphate to farnesene.

In some embodiments of each or any of the above or below mentioned embodiments, the microorganism is a bacteria selected from the genera consisting essentially of: Propionibacterium, Propionispira, Clostridium, Bacillus, Escherichia, Pelobacter, or Lactobacillus.

In some embodiments of each or any of the above or below mentioned embodiments, the microorganism is a eukaryote selected from the group consisting essentially of a yeast, filamentous fungi, protozoa, or algae.

In some embodiments of each or any of the above or below mentioned embodiments, the microorganism is from a genus selected from the group consisting of: Saccharomyces, Yarrowia, Hansenula, Pichia, Ashbya, and Candida.

The present disclosure provides methods of co-producing a terpene, succinate, and hydrogen from a fermentable carbon source comprising: providing a fermentable carbon source; expressing one or more exogenous polynucleotides in a microorganism that encode one or more enzymes in a pathway that catalyze a conversion of the fermentable carbon source to one or more intermediates in a pathway for production of terpene, succinate, and hydrogen; expressing one or more polynucleotides in a microorganism that encode one or more enzymes in a pathway that catalyze a conversion of one or more intermediates to terpene, succinate, and hydrogen; and contacting the fermentable carbon source with the microorganism, wherein the one or more intermediates in the pathway for the production of terpene include one or more intermediates in a mevalonate pathway, wherein the one or more intermediates in the pathway for the production of succinate include oxaloacetate, and wherein the co-production method is anaerobic.

In some embodiments of each or any of the above or below mentioned embodiments, the terpene is isoprene, farnesene, squalene, and/or bisabolene.

In some embodiments of each or any of the above or below mentioned embodiments, the one or more enzymes that catalyze the conversion of the fermentable carbon source to one or more intermediates in the pathway for the production of terpene, succinate, and hydrogen are set forth in Table 1.

In some embodiments of each or any of the above or below mentioned embodiments, the one or more enzymes that catalyze the conversion of the one or more intermediates to terpene, succinate, and hydrogen are set forth in Table 1.

In some embodiments of each or any of the above or below mentioned embodiments, the microorganism is a bacteria selected from the genera consisting essentially of: Propionibacterium, Propionispira, Clostridium, Bacillus, Escherichia, Pelobacter, or Lactobacillus.

In some embodiments of each or any of the above or below mentioned embodiments, the microorganism is a eukaryote selected from the group consisting essentially of a yeast, filamentous fungi, protozoa, or algae.

In some embodiments of each or any of the above or below mentioned embodiments, the microorganism is from a genus selected from the group consisting of: Saccharomyces, Yarrowia, Hansenula, Pichia, Ashbya, and Candida.

In some embodiments of each or any of the above or below mentioned embodiments, the fermentable carbon source is comprises sugarcane juice, sugarcane molasses, hydrolyzed starch, hydrolyzed lignocellulosic materials, glucose, sucrose, fructose, lactate, lactose, xylose, pyruvate, or glycerol in any form or mixture thereof.

In some embodiments of each or any of the above or below mentioned embodiments, the fermentable carbon source is a monosaccharide, oligosaccharide, or polysaccharide.

In some embodiments of each or any of the above or below mentioned embodiments, the terpene and at least one co-product are secreted by the microorganism into the fermentation media.

In some embodiments of each or any of the above or below mentioned embodiments, the methods may further comprise recovering terpene and at least a co-product from the fermentation media.

In some embodiments of each or any of the above or below mentioned embodiments, the fermentable carbon source is contacted with the microorganism prior to expressing in the microorganism the one or more polynucleotides that encode one or more enzymes in a pathway that catalyze a conversion of the fermentable carbon source to one or more intermediates in a pathway for the production of isoprene, succinate, and hydrogen and the one or more polynucleotides that encode one or more enzymes in a pathway that catalyze a conversion of the one or more intermediates to isoprene, succinate, and hydrogen.

In some embodiments of each or any of the above or below mentioned embodiments, isoprene, farnesene, succinate, and hydrogen are produced.

In some embodiments of each or any of the above or below mentioned embodiments, the fermentable carbon source is contacted with the microorganism after expressing in the microorganism the one or more polynucleotides that encode one or more enzymes in a pathway that catalyze a conversion of the fermentable carbon source to one or more intermediates in a pathway for the production of isoprene, succinate, and hydrogen and the one or more polynucleotides that encode one or more enzymes in a pathway that catalyze a conversion of the one or more intermediates to isoprene, succinate, and hydrogen.

The present disclosure also provides microorganisms comprising one or more polynucleotides that encode one or more enzymes in a pathway that catalyze a conversion of the fermentable carbon source to one or more intermediates in a pathway for the production of isoprene, succinate, and hydrogen and one or more polynucleotides that encode one or more enzymes in a pathway that catalyze a conversion of the one or more intermediates to isoprene, succinate, and hydrogen, wherein the one or more intermediates in the pathway for the production of terpene include one or more intermediates in a mevalonate pathway, and wherein the one or more intermediates in the pathway for the production of succinate include oxaloacetate.

In some embodiments of each or any of the above or below mentioned embodiments, the one or more enzymes that catalyze the conversion of the fermentable carbon source to one or more intermediates in the pathway for the production of terpene, succinate, and hydrogen are set forth in Table 1.

In some embodiments of each or any of the above or below mentioned embodiments, the one or more enzymes that catalyze the conversion of the one or more intermediates to terpene, succinate, and hydrogen are set forth in Table 1.

In some embodiments of each or any of the above or below mentioned embodiments, the microorganism is a bacteria selected from the genera consisting of: Burkholderia, Propionibacterium, Propionispira, Clostridium, Bacillus, Escherichia, Pelobacter, or Lactobacillus.

In some embodiments of each or any of the above or below mentioned embodiments, the microorganism is a eukaryote selected from the group consisting of yeast, filamentous fungi, protozoa, and algae.

In some embodiments of each or any of the above or below mentioned embodiments, the yeast is Saccharomyces cerevisiae, Zymomonas mobilis, or Pichia pastoris.

In some embodiments of each or any of the above or below mentioned embodiments, the microorganism has been genetically modified to express the one or more polynucleotides coding for enzymes in a pathway that catalyze a conversion of the fermentable carbon source to one or more intermediates in a pathway for the production of isoprene, succinate, and hydrogen and one or more polynucleotides coding for enzymes in a pathway that catalyze a conversion of the one or more intermediates to isoprene, succinate, and hydrogen.

These and other embodiments of the present disclosure will be disclosed in further detail herein below.

### BRIEF DESCRIPTION OF THE DRAWINGS

The foregoing summary, as well as the following detailed description of the disclosure, will be better understood when read in conjunction with the appended FIGURES. For the purpose of illustrating the disclosure, shown in the figures are embodiments which are presently preferred. It should be understood, however, that the disclosure is not limited to the precise arrangements, examples and instrumentalities shown.

FIG. 1 depicts an exemplary pathway for the co-production of a terpene (via a mevalonate pathway) and succinate via an oxaloacetate intermediate.

### DETAILED DESCRIPTION

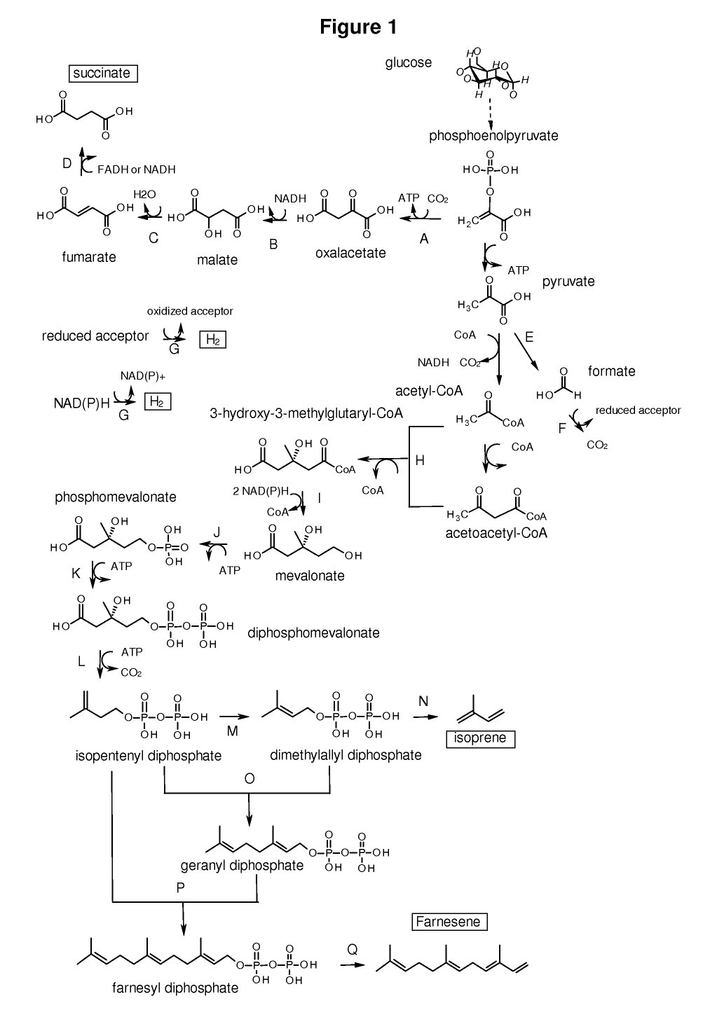

The present disclosure generally relates to microorganisms (e.g., non-naturally occurring microorganisms) that comprise a genetically modified pathway and uses of the microorganisms for the conversion of a fermentable carbon source to a terpene such as isoprene and succinate (see, FIG. 1). Such microorganisms may comprise one or more polynucleotides coding for enzymes that catalyze a conversion of a fermentable carbon source to a terpene, succinate, and hydrogen. In particular, the mevalonate (MVA) pathway (FIG. 1) has been modified for the biosynthesis of a terpene. For example, the microorganism may be modified to comprise one or more polynucleotides coding for enzymes that catalyze a conversion of one or more intermediates of the mevalonate pathway to one or more terpenes such as isoprene and/or farnesene. Optionally, the microorganism may be further modified to comprise one or more polynucleotides coding for enzymes that catalyze a conversion of an intermediate of Table 1 such as oxaloacetate to succinate.

The present disclosure provides methods of co-producing a terpene, succinate, and hydrogen from a fermentable carbon source comprising: providing a fermentable carbon source; expressing one or more exogenous polynucleotides in a microorganism that encode one or more enzymes in a pathway that catalyze a conversion of the fermentable carbon source to one or more intermediates in a pathway for production of terpene, succinate, and hydrogen; expressing one or more polynucleotides in a microorganism that encode one or more enzymes in a pathway that catalyze a conversion of one or more intermediates to terpene, succinate, and hydrogen; and contacting the fermentable carbon source with the microorganism, wherein the one or more intermediates in the pathway for the production of terpene include one or more intermediates in a mevalonate pathway, wherein the one or more intermediates in the pathway for the production of succinate include oxaloacetate, and wherein the co-production method is anaerobic. Such methods may comprise: providing a glucose source; expressing one or more exogenous polynucleotides in a microorganism that encode one or more enzymes in a pathway that catalyze a conversion of glucose to phosphoenolpyruvate, one or more polynucleotides coding for enzymes in a pathway that catalyze a conversion of phosphoenolpyruvate to oxaloacetate, one or more polynucleotides coding for enzymes in a pathway that catalyze a conversion of oxaloacetate to malate, one or more polynucleotides coding for enzymes in a pathway that catalyze a conversion of malate to fumarate, one or more polynucleotides coding for enzymes in a pathway that catalyze a conversion of fumarate to succinate, one or more polynucleotides coding for enzymes in a pathway that catalyze a conversion of pyruvate to acetyl-CoA and formate, one or more polynucleotides coding for enzymes in a pathway that catalyze a conversion of formate to CO₂ or formate to CO₂ and hydrogen, one or more polynucleotides coding for enzymes in a pathway that catalyze a conversion of NAD(P)H to NAD(P)⁺ and hydrogen, or a conversion of a reduced acceptor to CO₂ and hydrogen, one or more polynucleotides coding for enzymes in a pathway that catalyze a conversion of acetoacetyl-CoA and acetyl-CoA to 3-hydroxy-3-methylglutaryl-CoA, one or more polynucleotides coding for enzymes in a pathway that catalyze a conversion of 3-hydroxy-3-methylglutaryl-CoA to mevalonate, one or more polynucleotides coding for enzymes in a pathway that catalyze a conversion of mevalonate to phosphomevalonate, one or more polynucleotides coding for enzymes in a pathway that catalyze a conversion of phosphomevalonate to diphosphomevalonate, one or more polynucleotides coding for enzymes in a pathway that catalyze a conversion of diphosphomevalonate to isopentenyl diphosphate, one or more polynucleotides coding for enzymes in a pathway that catalyze a conversion of isopentenyl diphosphate to dimethylallyl diphosphate, one or more polynucleotides coding for enzymes in a pathway that catalyze a conversion of dimethylallyl diphosphate to isoprene, one or more polynucleotides coding for enzymes in a pathway that catalyze a conversion of dimethylallyl diphosphate and isopentenyl diphosphate to diphosphate and geranyl diphosphate, one or more polynucleotides coding for enzymes in a pathway that catalyze a conversion of geranyl diphosphate and isopentenyl diphosphate to diphosphate and farnesyl diphosphate, and/or one or more polynucleotides coding for enzymes in a pathway that catalyze a conversion of farnesyl diphosphate to farnesene; and contacting the glucose source with the microorganism, wherein the co-production method is anaerobic.

The present disclosure also provides microorganisms comprising one or more polynucleotides that encode one or more enzymes in a pathway that catalyze a conversion of the fermentable carbon source to one or more intermediates in a pathway for the production of isoprene, succinate, and hydrogen and one or more polynucleotides that encode one or more enzymes in a pathway that catalyze a conversion of the one or more intermediates to isoprene, succinate, and hydrogen, wherein the one or more intermediates in the pathway for the production of terpene include one or more intermediates in a mevalonate pathway, and wherein the one or more intermediates in the pathway for the production of succinate include oxaloacetate. Such microorganisms may comprise one or more polynucleotides coding for enzymes in a pathway that catalyze a conversion of phosphoenolpyruvate to oxaloacetate, one or more polynucleotides coding for enzymes in a pathway that catalyze a conversion of oxaloacetate to malate, one or more polynucleotides coding for enzymes in a pathway that catalyze a conversion of malate to fumarate, one or more polynucleotides coding for enzymes in a pathway that catalyze a conversion of fumarate to succinate, one or more polynucleotides coding for enzymes in a pathway that catalyze a conversion of pyruvate to acetyl-CoA and formate, one or more polynucleotides coding for enzymes in a pathway that catalyze a conversion of formate to CO₂ or formate to CO₂ and hydrogen, one or more polynucleotides coding for enzymes in a pathway that catalyze a conversion of NAD(P)H to NAD(P)⁺ and hydrogen, or a conversion of a reduced acceptor to CO₂ and hydrogen, one or more polynucleotides coding for enzymes in a pathway that catalyze a conversion of acetoacetyl-CoA and acetyl-CoA to 3-hydroxy-3-methylglutaryl-CoA, one or more polynucleotides coding for enzymes in a pathway that catalyze a conversion of 3-hydroxy-3-methylglutaryl-CoA to mevalonate, one or more polynucleotides coding for enzymes in a pathway that catalyze a conversion of mevalonate to phosphomevalonate, one or more polynucleotides coding for enzymes in a pathway that catalyze a conversion of phosphomevalonate to diphosphomevalonate, one or more polynucleotides coding for enzymes in a pathway that catalyze a conversion of diphosphomevalonate to isopentenyl diphosphate, one or more polynucleotides coding for enzymes in a pathway that catalyze a conversion of isopentenyl diphosphate to dimethylallyl diphosphate, one or more polynucleotides coding for enzymes in a pathway that catalyze a conversion of dimethylallyl diphosphate to isoprene, one or more polynucleotides coding for enzymes in a pathway that catalyze a conversion of dimethylallyl diphosphate and isopentenyl diphosphate to diphosphate and geranyl diphosphate, one or more polynucleotides coding for enzymes in a pathway that catalyze a conversion of geranyl diphosphate and isopentenyl diphosphate to diphosphate and farnesyl diphosphate, and/or one or more polynucleotides coding for enzymes in a pathway that catalyze a conversion of farnesyl diphosphate to farnesene.

This disclosure provides, in part, the discovery of novel enzymatic pathways including, for example, novel combinations of enzymatic pathways, for the production of a terpene such as isoprene, succinate, and hydrogen from a carbon source (e.g., a fermentable carbon source).

By coupling pathways for the co-production of a terpene such as isoprene and succinate it is possible to achieve a redox neutral and ATP positive balance, allowing for an anaerobic process as indicated below:

1.5 glucose+4NAD(P)+→Isoprene+4NAD(P)H+4CO₂

0.5 Glucose+1CO₂+1ADP→Succinate+1NAD++1ATP(×4)

Overall: 3.5 Glucose→Isoprene+4 Succinate+4ATP

This stoichiometry yields a maximum theoretical yield of 11% (g/g) of isoprene and 75% of succinate. Thus, the production of isoprene is almost seven times higher than isoprene. However, the market for synthetic isoprene is higher than 1 million ton per year in comparison to no less than 50 thousand tons per year of succinate. Even though the market of succinic acid is expect to grow in 20% per year, a pathway with a suitable proportion of isoprene and succinate is required. In this regard, pyruvate formate lyase, formate hydrogen lyase, and/or dehydrogenases may be expressed in order to increase isoprene yields and allow hydrogen co-production (e.g., hydrogen may be formed from the reversible conversion of two protons and two electrons catalyzed by a hydrogenase). Accordingly, by regulating the expression of those genes (reactions E and/or F of FIG. 1) for the conversion of hydrogen, different yields of isoprene, succinate and hydrogen could be reached including, for example, as shown below:

3 glucose→Isoprene+3 Succinate+1H₂+1CO₂+3ATP i)
(This stoichiometry yields a maximum theoretical yield of 13% (g/g) of isoprene and 66% of succinate).

2.5 glucose→Isoprene+2 Succinate+2H₂+2CO₂+2ATP ii)
(This stoichiometry yields a maximum theoretical yield of 15% (g/g) of isoprene and 52% of succinate).

2 glucose→Isoprene+1 Succinate+2H₂+2 CO₂+1 ATP iii)
(This stoichiometry yields a maximum theoretical yield of 19% (g/g) of isoprene and 33% of succinate).

In some embodiments, hydrogen production could be used as energy source or used for the hydrogenation of succinic acid to 1,4-butanediol.

The pathways disclosed herein are advantageous over prior known enzymatic pathways for the production of a terpene such as isoprene and succinate in that the enzymatic pathways disclosed herein are anaerobic thereby reducing the risk of an explosion during the manufacture of the terpene. Additionally, the terpene and the co-product produced by the processes disclosed herein are not diluted by O₂ and N₂ thus preventing both costly and time-consuming purification of the produced terpene and succinate.

While it is possible to use aerobic processes to produce a terpene such as isoprene, anaerobic processes are preferred due to the risk incurred when olefins (which are explosive by nature) are mixed with oxygen during the fermentation process. Moreover, the supplementation of oxygen and nitrogen in the fermenter requires additional investment for aerobic process and another additional investment for the purification from the nitrogen from the isoprene. The presence of oxygen can also catalyze the polymerization of a terpene, and promotes the growth of aerobic contaminants in the fermenter broth as well. Aerobic fermentation processes for the production of terpene present several drawbacks at industrial scale, such as the facts that: (i) greater biomass is obtained reducing overall yields on carbon; (ii) the presence and oxygen favors the growth of contaminants (Weusthuis et al. (2010) Trends in Biotechnology, 29 (4): 153-158) and (iii) the mixture of oxygen and gaseous compounds such as isoprene poses serious risks of explosion, (iv) the oxygen can catalyze the unwanted polymerization of the olefin, and (v) fermentation and purification in aerobic conditions are more expensive.

In addition to an anaerobic process for production of a terpene, the method disclosed provides a method by which a genetically modified microorganism can produce two products simultaneously (co-production): in this case, a terpene along with succinate.

In some embodiments, the ratio of grams of the produced terpene and succinate to grams of the fermentable carbon source is 0.01-0.98.

As used herein, the term “acceptor” includes but is not limited to NAD+ or NADP+ or quinone, or oxidized cytochrome c. Additionally, as used herein the term “reduced acceptor” includes but is not limited to NADH or NADPH or quinol or reduced cytochrome c.

As used herein, the term “biological activity” or “functional activity,” when referring to a protein, polypeptide or peptide, may mean that the protein, polypeptide or peptide exhibits a functionality or property that is useful as relating to some biological process, pathway or reaction. Biological or functional activity can refer to, for example, an ability to interact or associate with (e.g., bind to) another polypeptide or molecule, or it can refer to an ability to catalyze or regulate the interaction of other proteins or molecules (e.g., enzymatic reactions).

As used herein, the term “culturing” may refer to growing a population of cells, e.g., microbial cells, under suitable conditions for growth, in a liquid or on solid medium.

As used herein, the term “derived from” may encompass the terms originated from, obtained from, obtainable from, isolated from, and created from, and generally indicates that one specified material finds its origin in another specified material or has features that can be described with reference to the another specified material.

As used herein, “exogenous polynucleotide” refers to any deoxyribonucleic acid that originates outside of the microorganism.

As used herein, the term “expression vector” may refer to a DNA construct containing a polynucleotide or nucleic acid sequence encoding a polypeptide or protein, such as a DNA coding sequence (e.g., gene sequence) that is operably linked to one or more suitable control sequence(s) capable of affecting expression of the coding sequence in a host. Such control sequences include a promoter to affect transcription, an optional operator sequence to control such transcription, a sequence encoding suitable mRNA ribosome binding sites, and sequences which control termination of transcription and translation. The vector may be a plasmid, a phage particle, or simply a potential genomic insert. Once transformed into a suitable host, the vector may replicate and function independently of the host genome (e.g., independent vector or plasmid), or may, in some instances, integrate into the genome itself (e.g., integrated vector). The plasmid is the most commonly used form of expression vector. However, the disclosure is intended to include such other forms of expression vectors that serve equivalent functions and which are, or become, known in the art.

As used herein, the term “expression” may refer to the process by which a polypeptide is produced based on a nucleic acid sequence encoding the polypeptides (e.g., a gene). The process includes both transcription and translation.

As used herein, the term “gene” may refer to a DNA segment that is involved in producing a polypeptide or protein (e.g., fusion protein) and includes regions preceding and following the coding regions as well as intervening sequences (introns) between individual coding segments (exons).

As used herein, the term “heterologous,” with reference to a nucleic acid, polynucleotide, protein or peptide, may refer to a nucleic acid, polynucleotide, protein or peptide that does not naturally occur in a specified cell, e.g., a host cell. It is intended that the term encompass proteins that are encoded by naturally occurring genes, mutated genes, and/or synthetic genes. In contrast, the term homologous, with reference to a nucleic acid, polynucleotide, protein or peptide, refers to a nucleic acid, polynucleotide, protein or peptide that occurs naturally in the cell.

As used herein, the term a “host cell” may refer to a cell or cell line, including a cell such as a microorganism which a recombinant expression vector may be transfected for expression of a polypeptide or protein (e.g., fusion protein). Host cells include progeny of a single host cell, and the progeny may not necessarily be completely identical (in morphology or in total genomic DNA complement) to the original parent cell due to natural, accidental, or deliberate mutation. A host cell may include cells transfected or transformed in vivo with an expression vector.

As used herein, the term “introduced,” in the context of inserting a nucleic acid sequence or a polynucleotide sequence into a cell, may include transfection, transformation, or transduction and refers to the incorporation of a nucleic acid sequence or polynucleotide sequence into a eukaryotic or prokaryotic cell wherein the nucleic acid sequence or polynucleotide sequence may be incorporated into the genome of the cell (e.g., chromosome, plasmid, plastid, or mitochondrial DNA), converted into an autonomous replicon, or transiently expressed.

As used herein, “isoprene” is intended to mean 2-methyl-1,3-butadiene, is a common organic compound with molecular formula C5H8, a general formula CH₂═C(CH₃)CH═CH₂ and a molecular mass of 68.12 g/mol.

As used herein, the term “non-naturally occurring” when used in reference to a microbial organism or microorganism of the invention is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. A non-naturally occurring microbial organisms of the disclosure can contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely. Those skilled in the art will understand that the genetic alterations, including metabolic modifications exemplified herein, are described with reference to a suitable host organism such as E. coli and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or nonorthologous gene displacements.

As used herein, the term “operably linked” may refer to a juxtaposition or arrangement of specified elements that allows them to perform in concert to bring about an effect. For example, a promoter may be operably linked to a coding sequence if it controls the transcription of the coding sequence.

As used herein, the term “a promoter” may refer to a regulatory sequence that is involved in binding RNA polymerase to initiate transcription of a gene. A promoter may be an inducible promoter or a constitutive promoter. An inducible promoter is a promoter that is active under environmental or developmental regulatory conditions.

As used herein, the term “a polynucleotide” or “nucleic acid sequence” may refer to a polymeric form of nucleotides of any length and any three-dimensional structure and single- or multi-stranded (e.g., single-stranded, double-stranded, triple-helical, etc.), which contain deoxyribonucleotides, ribonucleotides, and/or analogs or modified forms of deoxyribonucleotides or ribonucleotides, including modified nucleotides or bases or their analogs. Such polynucleotides or nucleic acid sequences may encode amino acids (e.g., polypeptides or proteins such as fusion proteins). Because the genetic code is degenerate, more than one codon may be used to encode a particular amino acid, and the present disclosure encompasses polynucleotides which encode a particular amino acid sequence. Any type of modified nucleotide or nucleotide analog may be used, so long as the polynucleotide retains the desired functionality under conditions of use, including modifications that increase nuclease resistance (e.g., deoxy, 2′-O-Me, phosphorothioates, etc.). Labels may also be incorporated for purposes of detection or capture, for example, radioactive or nonradioactive labels or anchors, e.g., biotin. The term polynucleotide also includes peptide nucleic acids (PNA). Polynucleotides may be naturally occurring or non-naturally occurring. The terms polynucleotide, nucleic acid, and oligonucleotide are used herein interchangeably. Polynucleotides may contain RNA, DNA, or both, and/or modified forms and/or analogs thereof. A sequence of nucleotides may be interrupted by non-nucleotide components. One or more phosphodiester linkages may be replaced by alternative linking groups. These alternative linking groups include, but are not limited to, embodiments wherein phosphate is replaced by P(O)S (thioate), P(S)S (dithioate), (O)NR₂ (amidate), P(O)R, P(O)OR′, COCH₂ (formacetal), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. Polynucleotides may be linear or circular or comprise a combination of linear and circular portions.

As used herein, the term a “protein” or “polypeptide” may refer to a composition comprised of amino acids and recognized as a protein by those of skill in the art. The conventional one-letter or three-letter code for amino acid residues is used herein. The terms protein and polypeptide are used interchangeably herein to refer to polymers of amino acids of any length, including those comprising linked (e.g., fused) peptides/polypeptides (e.g., fusion proteins). The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, related proteins, polypeptides or peptides may encompass variant proteins, polypeptides or peptides. Variant proteins, polypeptides or peptides differ from a parent protein, polypeptide or peptide and/or from one another by a small number of amino acid residues. In some embodiments, the number of different amino acid residues is any of about 1, 2, 3, 4, 5, 10, 20, 25, 30, 35, 40, 45, or 50. In some embodiments, variants differ by about 1 to about 10 amino acids. Alternatively or additionally, variants may have a specified degree of sequence identity with a reference protein or nucleic acid, e.g., as determined using a sequence alignment tool, such as BLAST, ALIGN, and CLUSTAL (see, infra). For example, variant proteins or nucleic acid may have at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% amino acid sequence identity with a reference sequence.

As used herein, the term “recovered,” “isolated,” “purified,” and “separated” may refer to a material (e.g., a protein, peptide, nucleic acid, polynucleotide or cell) that is removed from at least one component with which it is naturally associated. For example, these terms may refer to a material which is substantially or essentially free from components which normally accompany it as found in its native state, such as, for example, an intact biological system.

As used herein, the term “recombinant” may refer to nucleic acid sequences or polynucleotides, polypeptides or proteins, and cells based thereon, that have been manipulated by man such that they are not the same as nucleic acids, polypeptides, and cells as found in nature. Recombinant may also refer to genetic material (e.g., nucleic acid sequences or polynucleotides, the polypeptides or proteins they encode, and vectors and cells comprising such nucleic acid sequences or polynucleotides) that has been modified to alter its sequence or expression characteristics, such as by mutating the coding sequence to produce an altered polypeptide, fusing the coding sequence to that of another coding sequence or gene, placing a gene under the control of a different promoter, expressing a gene in a heterologous organism, expressing a gene at decreased or elevated levels, expressing a gene conditionally or constitutively in manners different from its natural expression profile, and the like.

As used herein, the term “selective marker” or “selectable marker” may refer to a gene capable of expression in a host cell that allows for ease of selection of those hosts containing an introduced nucleic acid sequence, polynucleotide or vector. Examples of selectable markers include but are not limited to antimicrobial substances (e.g., hygromycin, bleomycin, or chloramphenicol) and/or genes that confer a metabolic advantage, such as a nutritional advantage, on the host cell.

As used herein, the term “substantially similar” and “substantially identical” in the context of at least two nucleic acids, polynucleotides, proteins or polypeptides may mean that a nucleic acid, polynucleotide, protein or polypeptide comprises a sequence that has at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% sequence identity, in comparison with a reference (e.g., wild-type) nucleic acid, polynucleotide, protein or polypeptide. Sequence identity may be determined using known programs such as BLAST, ALIGN, and CLUSTAL using standard parameters. (See, e.g., Altshul et al. (1990) J. Mol. Biol. 215:403-410; Henikoff et al. (1989) Proc. Natl. Acad. Sci. 89:10915; Karin et al. (1993) Proc. Natl. Acad. Sci. 90:5873; and Higgins et al. (1988) Gene 73:237). Software for performing BLAST analyses is publicly available through the National Center for Biotechnology Information. Also, databases may be searched using FASTA (Person et al. (1988) Proc. Natl. Acad. Sci. 85:2444-2448.) In some embodiments, substantially identical polypeptides differ only by one or more conservative amino acid substitutions. In some embodiments, substantially identical polypeptides are immunologically cross-reactive. In some embodiments, substantially identical nucleic acid molecules hybridize to each other under stringent conditions (e.g., within a range of medium to high stringency).

As used herein, the term “succinic acid” (butanedioic acid, 1,2-ethanedicarboxylic acid, HO2CCH2CH2CO2H, CAS110-15-6) is intended to mean a C4 dicarboxylic acid, molecular weight 1108.09 g/mol.

As used herein, the term “terpene” refers to a product having the formula (C₅H₈)ₙ. where n is 1 (i.e., isoprene), 2, 3, 4, 5, 6, 7, 8, 9, 10, or more. Terpenes may be classified by the number of isoprene units in the molecule; a prefix in the name indicates the number of terpene units needed to assemble the molecule.

Hemiterpenes consist of a single isoprene unit. Isoprene itself is considered the only hemiterpene, but oxygen-containing derivatives such as prenol and isovalericacid arehemiterpenoids.

Monoterpenes consist of two isoprene units and have the molecular formula C₁₀H₁₆. Examples of monoterpenes are: geraniol, limonene and terpeneol.

Sesquiterpenes consist of three isoprene units and have the molecular formula C₁₅H₂₄. Examples of sesquiterpenes are: humulene, farnesenes, farnesol.

Diterpenes are composed of four isoprene units and have the molecular formula C₂₀H₃₂. They derive from geranylgeranyl pyrophosphate. Examples of diterpenes are cafestol, kahweol, cembrene and taxadiene (precursor of taxol). Diterpenes also form the basis for biologically important compounds such as retinol, retinal, and phytol. They are known to be antimicrobial and antiinflammatory.

Sesterterpenes, terpenes having 25 carbons and five isoprene units, are rare relative to the other sizes. (The sester-prefix means half to three, i.e. two and a half.) An example of a sesterterpene is geranylfarnesol.

Triterpenes consist of six isoprene units and have the molecular formula C₃₀H₄₈. The linear triterpenesqualene, the major constituent of shark liver oil, is derived from the reductive coupling of two molecules of farnesyl pyrophosphate. Squalene is then processed biosynthetically to generate either lanosterol or cycloartenol, the structural precursors to all the steroids.

Sesquarterpenes are composed of seven isoprene units and have the molecular formula C₃₅H₅₆. Sesquarterpenes are typically microbial in their origin. Examples of sesquarterpenes are ferrugicadiol and tetraprenylcurcumene.

Tetraterpenes contain eight isoprene units and have the molecular formula C₄₀H₆₄. Biologically important tetraterpenes include the acyclic lycopene, the monocyclic gamma-carotene, and the bicyclic alpha- and beta-carotenes.

Polyterpenes consist of long chains of many isoprene units. Natural rubber consists of polyisoprene in which the double bonds are cis. Some plants produce a polyisoprene with trans double bonds, known as gutta-percha.

Norisoprenoids, such as the C₁₃-norisoprenoids 3-oxo-α-ionol present in Muscat of Alexandria leaves and 7,8-dihydroionone derivatives, such as megastigmane-3,9-diol and 3-oxo-7,8-dihydro-α-ionol found in Shiraz leaves (both grapes in the species Vitisvinifera) or wine (responsible for some of the spice notes in Chardonnay), can be produced by fungal peroxydase or glycosidases.

As used herein, the term “transfection” or “transformation” may refer to the insertion of an exogenous nucleic acid or polynucleotide into a host cell. The exogenous nucleic acid or polynucleotide may be maintained as a non-integrated vector, for example, a plasmid, or alternatively, may be integrated into the host cell genome. The term transfecting or transfection is intended to encompass all conventional techniques for introducing nucleic acid or polynucleotide into host cells. Examples of transfection techniques include, but are not limited to, calcium phosphate precipitation, DEAE-dextran-mediated transfection, lipofection, electroporation, and microinjection.

As used herein, the term “transformed,” “stably transformed,” and “transgenic” may refer to a cell that has a non-native (e.g., heterologous) nucleic acid sequence or polynucleotide sequence integrated into its genome or as an episomal plasmid that is maintained through multiple generations.

As used herein, the term “vector” may refer to a polynucleotide sequence designed to introduce nucleic acids into one or more cell types. Vectors include cloning vectors, expression vectors, shuttle vectors, plasmids, phage particles, single and double stranded cassettes and the like.

As used herein, the term “wild-type,” “native,” or “naturally-occurring” proteins may refer to those proteins found in nature. The terms wild-type sequence refers to an amino acid or nucleic acid sequence that is found in nature or naturally occurring. In some embodiments, a wild-type sequence is the starting point of a protein engineering project, for example, production of variant proteins.

Unless defined otherwise herein, all technical and scientific terms used herein have the same meaning as commonly understood by one of ordinary skill in the art to which this disclosure belongs. Singleton, et al., Dictionary of Microbiology and Molecular Biology, second ed., John Wiley and Sons, New York (1994), and Hale & Markham, The Harper Collins Dictionary of Biology, Harper Perennial, N.Y. (1991) provide one of skill with a general dictionary of many of the terms used in this disclosure.

Numeric ranges provided herein are inclusive of the numbers defining the range.

Unless otherwise indicated, nucleic acids sequences are written left to right in 5′ to 3′ orientation; amino acid sequences are written left to right in amino to carboxy orientation, respectively.

While the present disclosure is capable of being embodied in various forms, the description below of several embodiments is made with the understanding that the present disclosure is to be considered as an exemplification of the disclosure, and is not intended to limit the disclosure to the specific embodiments illustrated. Headings are provided for convenience only and are not to be construed to limit the disclosure in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present disclosure.

### Modification of Microorganism

A microorganism may be modified (e.g., genetically engineered) by any method known in the art to comprise and/or express one or more polynucleotides coding for enzymes in a pathway that catalyze a conversion of a fermentable carbon source to one or more intermediates in a pathway for the co-production of a terpene such as isoprene, succinate, and hydrogen. Such enzymes may include any of those enzymes as are forth in Table 1. For example, the microorganism may be modified to comprise one or more polynucleotides coding for enzymes that catalyze a conversion of one or more intermediates of the mevalonate pathway to one or more terpenes such as isoprene and/or farnesene. Optionally, the microorganism may be further modified to comprise one or more polynucleotides coding for enzymes that catalyze a conversion of an intermediate of Table 1 such as oxaloacetate to succinate.

In some embodiments, the one or more polynucleotides coding for enzymes in a pathway that catalyze a conversion of one or more intermediates of the mevalonate pathway to one or more terpenes including, for example, isoprene and/or farnesene and a conversion of oxaloacetate to succinate include:

one or more polynucleotides coding for enzymes in a pathway that catalyze a conversion of phophoenolpyruvate, ADP, and CO₂ to oxaloacetate (e.g., a PEP carboxykinase),

one or more polynucleotides coding for enzymes in a pathway that catalyze a conversion of oxaloacetate and NADH to malate and NAD+ (e.g., a malate dehydrogenase);

one or more polynucleotides coding for enzymes in a pathway that catalyze a conversion of malate to fumarate and H₂O (e.g., a fumarate hydratase);

one or more polynucleotides coding for enzymes in a pathway that catalyze a conversion of fumarate and a reduced acceptor to succinate and an acceptor (e.g., a fumarate dehydrogenase or succinate dehydrogenase);

one or more polynucleotides coding for enzymes in a pathway that catalyze a conversion of pyruvate to acetyl-CoA and formate (e.g., a pyruvate formate lyase);

one or more polynucleotides coding for enzymes in a pathway that catalyze a conversion of formate and NAD+ to CO₂ and NADH, or formate to CO₂ and H₂ (e.g., a formate dehydrogenase or a formate hydrogen lyase);

one or more polynucleotides coding for enzymes in a pathway that catalyze a conversion of NAD(P)H to NAD(P)⁺ and H₂, or a reduced acceptor to CO₂ and H₂ (e.g., a hydrogen dehydrogenase or hydrogen dehydrogenase (NADP+);

one or more polynucleotides coding for enzymes in a pathway that catalyze a conversion of acetoacetyl-CoA, acetyl-CoA, and H₂O to 3-hydroxy-3-methylglutaryl-CoA (e.g., a hydroxymethylglutaryl-CoA synthase);

one or more polynucleotides coding for enzymes in a pathway that catalyze a conversion of 3-hydroxy-3-methylglutaryl-CoA and two NAD(P)H to mevalonate, CoA, two NADP+, and two H+(e.g., a hydroxymethylglutaryl-CoA reductase);

one or more polynucleotides coding for enzymes in a pathway that catalyze a conversion of mevalonate and ATP to phosphomevalonate and ADP (e.g., a mevalonate kinase);

one or more polynucleotides coding for enzymes in a pathway that catalyze a conversion of phosphomevalonate to diphosphomevalonate (e.g., a phosphomevalonate kinase);

one or more polynucleotides coding for enzymes in a pathway that catalyze a conversion of diphosphomevalonate and ATP to isopentenyl diphosphate, CO₂ and ADP (e.g., a diphosphomevalonate decarboxylase);

one or more polynucleotides coding for enzymes in a pathway that catalyze a conversion of isopentenyl diphosphate to dimethylallyl diphosphate (e.g., an isopentenyl diphosphate delta-isomerase);

one or more polynucleotides coding for enzymes in a pathway that catalyze a conversion of dimethylallyl diphosphate to isoprene (e.g., an isoprene synthase);

one or more polynucleotides coding for enzymes in a pathway that catalyze a conversion of dimethylallyl diphosphate and isopentenyl diphosphate to diphosphate and geranyl diphosphate (e.g., a geranyl-diphosphate synthase);

one or more polynucleotides coding for enzymes in a pathway that catalyze a conversion of geranyl diphosphate and isopentenyl diphosphate to diphosphate and farnesyl diphosphate (e.g., a farnesyl pyrophosphate synthase); and/or

one or more polynucleotides coding for enzymes in a pathway that catalyze a conversion of farnesyl diphosphate to farnesene (e.g., a farnesene synthase).

Exemplary enzymes (e.g., those that convert oxaloacetate to succinate, and one or more intermediates of the mevalonate pathway to one or more terpenes such as isoprene and/or farnesene, enzyme substrates) and enzyme reaction products, are presented in Table 1 below. The enzyme reference identifier listed in Table 1 correlates with the enzyme numbering used in FIG. 1, which schematically represents the enzymatic conversion of a fermentable carbon source to one or more terpenes and succinate.

TABLE 1
Co-production of one or more terpenes via mevalonate pathway intermediates, succinate
via an oxaloacetate intermediate and hydrogen via a reduced acceptor.

| Enzyme | | E.C. | |
|---|---|---|---|
| Ref. | Enzyme Name | number | Mediated Conversion |
| A. | PEP carboxykinase | 4.1.1.49 | phosphoenolpyruvate + ADP + CO₂ → |
| | | | oxaloacetate + ATP |
| B. | malate dehydrogenase | 1.1.1.37 | oxaloacetate + NADH → malate + |
| | | | NAD+ |
| C. | fumarate hydratase | 4.2.1.2 | malate → fumarate + H₂O |
| D. | fumarate dehydrogenase or | 1.3.99.1 or | fumarate + reduced acceptor→ |
| | succinate dehydrogenase or | 1.3.5.1 or | succinate + acceptor |
| | Fumarate reductase | 1.3.1.6 | |
| E | pyruvate formate lyase | 2.3.1.54 | Pyruvate → Acetyl-CoA + formate |
| F. | Formate dehydrogenase or | 1.2.1.2 | Formate + NAD+ → CO₂ + NADH |
| | Formate hydrogen lyase | 1.1.99.33 | Formate → CO₂ + reduced acceptor + |
| | | | H₂ |
| G. | Hydrogen Dehydrogenase | 1.12.1.2 or | NAD(P)H → NAD(P)⁺ + H₂ |
| | or Hydrogen | 1.12.1.3 or | reduced acceptor → CO₂ + H₂ |
| | Dehydrogenase (NADP+) | 1.12.7 or | |
| | Other acceptors as iron | 1.12.98 | |
| | sulfur hydrogenase | 1.12.99 | |
| H. | hydroxymethylglutaryl-CoA | 2.3.3.10 | acetoacetyl-CoA + acetyl-CoA + H₂O → |
| | synthase | | 3-hydroxy-3-methylglutaryl-CoA |
| I. | hydroxymethylglutaryl-CoA | 1.1.1.88 or | 3-hydroxy-3-methylglutaryl-CoA + 2 |
| | reductase | 1.1.1.34 | NAD(P)H → mevalonate + CoA + 2 |
| | | | NADP + 2 H+ |
| J. | mevalonate kinase | 2.7.1.36 | mevalonate + ATP → |
| | | | phosphomevalonate + ADP |
| K. | phophomevalonate kinase | 2.7.4.2 | phosphomevaloante + ATP → |
| | | | diphosphomevalonate + ADP |
| L. | diphosphomevalonate | 4.1.1.33 | diphosphomevalonate + ATP → |
| | decarboxylase | | isopentenyl-diphosphate + CO₂ + ADP |
| M. | isopentenyl-diphosphate | 5.3.3.2 | isopentenyl diphosphate → |
| | delta-isomerase | | dimethylallyl diphosphate |
| N. | isoprene synthase | 4.2.3.27 | dimethylallyl diphosphate → isoprene + |
| | | | diphosphate |
| O. | geranyl-diphosphate | 2.5.1.1 | dimethylallyl diphosphate + isopentenyl |
| | synthase | | diphosphate → diphosphate + geranyl |
| | | | diphosphate |
| P. | farnesyl pyrophosphate | 2.5.1.10 | geranyl diphosphate + Isopentenyl |
| | synthase | | diphosphate → diphosphate + farnesyl |
| | | | diphosphate |
| Q. | farnesene synthase | 4.2.3.46 or | farnesyl diphosphate → farnesene + |
| | | 4.2.3.47 | diphosphate |

In some embodiments, the disclosure contemplates the modification (e.g., engineering) of one or more of the enzymes provided herein. Such modification may be performed to redesign the substrate specificity of the enzyme and/or to modify (e.g., reduce) its activity against others substrates in order to increase its selectivity for a given substrate. Additionally or alternatively, one or more enzymes as provided herein may be engineered to alter (e.g., enhance including, for example, increase its catalytic activity or its substrate specificity) one or more of its properties.

The one or more enzymes are expressed in a microorganism selected from an archea, bacteria, or eukaryote. In some embodiments, the bacteria is a Propionibacterium, Propionispira, Clostridium, Bacillus, Escherichia, Pelobacter, or Lactobacillus including, for example, Pelobacter propionicus, Clostridium propionicum, Clostridium acetobutylicum, Lactobacillus, Propionibacterium acidipropionici or Propionibacterium freudenreichii. In some embodiments, the eukaryote is a yeast, filamentous fungi, protozoa, or algae. In some embodiments, the yeast is Saccharomyces cerevisiae or Pichia pastoris.

In some embodiments, sequence alignment and comparative modeling of proteins may be used to alter one or more of the enzymes disclosed herein. Homology modeling or comparative modeling refers to building an atomic-resolution model of the desired protein from its primary amino acid sequence and an experimental three-dimensional structure of a similar protein. This model may allow for the enzyme substrate binding site to be defined, and the identification of specific amino acid positions that may be replaced to other natural amino acid in order to redesign its substrate specificity.

Variants or sequences having substantial identity or homology with the polynucleotides encoding enzymes as disclosed herein may be utilized in the practice of the disclosure. Such sequences can be referred to as variants or modified sequences. That is, a polynucleotide sequence may be modified yet still retain the ability to encode a polypeptide exhibiting the desired activity. Such variants or modified sequences are thus equivalents. Generally, the variant or modified sequence may comprise at least about 40%-60%, preferably about 60%-80%, more preferably about 80%-90%, and even more preferably about 90%-95% sequence identity with the native sequence.

The microorganism may be modified by genetic engineering techniques (i.e., recombinant technology), classical microbiological techniques, or a combination of such techniques and can also include naturally occurring genetic variants to produce a genetically modified microorganism. Some of such techniques are generally disclosed, for example, in Sambrook et al., 1989, Molecular Cloning: A Laboratory Manual, Cold Spring Harbor Labs Press.

A genetically modified microorganism may include a microorganism in which a polynucleotide has been inserted, deleted or modified (i.e., mutated; e.g., by insertion, deletion, substitution, and/or inversion of nucleotides), in such a manner that such modifications provide the desired effect of expression (e.g., over-expression) of one or more enzymes as provided herein within the microorganism. Genetic modifications which result in an increase in gene expression or function can be referred to as amplification, overproduction, overexpression, activation, enhancement, addition, or up-regulation of a gene. Addition of cloned genes to increase gene expression can include maintaining the cloned gene(s) on replicating plasmids or integrating the cloned gene(s) into the genome of the production organism. Furthermore, increasing the expression of desired cloned genes can include operatively linking the cloned gene(s) to native or heterologous transcriptional control elements.

Where desired, the expression of one or more of the enzymes provided herein is under the control of a regulatory sequence that controls directly or indirectly the enzyme expression in a time-dependent fashion during the fermentation.

In some embodiments, a microorganism is transformed or transfected with a genetic vehicle, such as an expression vector comprising an exogenous polynucleotide sequence coding for the enzymes provided herein.

Polynucleotide constructs prepared for introduction into a prokaryotic or eukaryotic host may typically, but not always, comprise a replication system (i.e. vector) recognized by the host, including the intended polynucleotide fragment encoding the desired polypeptide, and may preferably, but not necessarily, also include transcription and translational initiation regulatory sequences operably linked to the polypeptide-encoding segment. Expression systems (expression vectors) may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, mRNA stabilizing sequences, nucleotide sequences homologous to host chromosomal DNA, and/or a multiple cloning site. Signal peptides may also be included where appropriate, preferably from secreted polypeptides of the same or related species, which allow the protein to cross and/or lodge in cell membranes or be secreted from the cell.

The vectors can be constructed using standard methods (see, e.g., Sambrook et al., Molecular Biology: A Laboratory Manual, Cold Spring Harbor, N.Y. 1989; and Ausubel, et al., Current Protocols in Molecular Biology, Greene Publishing, Co. N.Y., 1995).

The manipulation of polynucleotides that encode the enzymes disclosed herein is typically carried out in recombinant vectors. Numerous vectors are publicly available, including bacterial plasmids, bacteriophage, artificial chromosomes, episomal vectors and gene expression vectors, which can all be employed. A vector may be selected to accommodate a polynucleotide encoding a protein of a desired size. Following recombinant modification of a selected vector, a suitable host cell is transfected or transformed with the vector. Host cells may be prokaryotic, such as any of a number of bacterial strains, or may be eukaryotic, such as yeast or other fungal cells, insect or amphibian cells, or mammalian cells including, for example, rodent, simian or human cells. Each vector contains various functional components, which generally include a cloning site, an origin of replication and at least one selectable marker gene. A vector may additionally possess one or more of the following elements: an enhancer, promoter, and transcription termination and/or other signal sequences. Such sequence elements may be optimized for the selected host species (e.g. humanized) Such sequence elements may be positioned in the vicinity of the cloning site, such that they are operatively linked to the gene encoding a preselected enzyme.

Vectors, including cloning and expression vectors, may contain nucleic acid sequences that enable the vector to replicate in one or more selected host cells. For example, the sequence may be one that enables the vector to replicate independently of the host chromosomal DNA and may include origins of replication or autonomously replicating sequences. Such sequences are well known for a variety of bacteria, yeast and viruses. For example, the origin of replication from the plasmid pBR322 is suitable for most Gram-negative bacteria, the 2 micron plasmid origin is suitable for yeast, and various viral origins (e.g. SV 40, adenovirus) are useful for cloning vectors in mammalian cells. Generally, the origin of replication is not needed for mammalian expression vectors unless these are used in mammalian cells able to replicate high levels of DNA, such as COS cells.

A cloning or expression vector may contain a selection gene (also referred to as a selectable marker). This gene encodes a protein necessary for the survival or growth of transformed host cells in a selective culture medium. Host cells not transformed with the vector containing the selection gene will therefore not survive in the culture medium. Typical selection genes encode proteins that confer resistance to antibiotics and other toxins, e.g. ampicillin, neomycin, methotrexate, hygromycin, thiostrepton, apramycin or tetracycline, complement auxotrophic deficiencies, or supply critical nutrients not available in the growth media.

The replication of vectors may be performed in E. coli (e.g., strain TB1 or TG1, DH5α, DH10β, JM110). An E. coli-selectable marker, for example, the β-lactamase gene that confers resistance to the antibiotic ampicillin, may be of use. These selectable markers can be obtained from E. coli plasmids, such as pBR322 or a pUC plasmid such as pUC18 or pUC19, or pUC119.

Expression vectors may contain a promoter that is recognized by the host organism. The promoter may be operably linked to a coding sequence of interest. Such a promoter may be inducible or constitutive. Polynucleotides are operably linked when the polynucleotides are in a relationship permitting them to function in their intended manner.

Promoters suitable for use with prokaryotic hosts may include, for example, the α-lactamase and lactose promoter systems, alkaline phosphatase, the tryptophan (trp) promoter system, the erythromycin promoter, apramycin promoter, hygromycin promoter, methylenomycin promoter and hybrid promoters such as the tac promoter. Moreover, host constitutive or inducible promoters may be used. Promoters for use in bacterial systems will also generally contain a Shine-Dalgarno sequence operably linked to the coding sequence.

Viral promoters obtained from the genomes of viruses include promoters from polyoma virus, fowlpox virus, adenovirus (e.g., Adenovirus 2 or 5), herpes simplex virus (thymidine kinase promoter), bovine papilloma virus, avian sarcoma virus, cytomegalovirus, retrovirus (e.g., MoMLV, or RSV LTR), Hepatitis-B virus, Myeloproliferative sarcoma virus promoter (MPSV), VISNA, and Simian Virus 40 (SV40). Heterologous mammalian promoters include, e.g., the actin promoter, immunoglobulin promoter, heat-shock protein promoters.

The early and late promoters of the SV40 virus are conveniently obtained as a restriction fragment that also contains the SV40 viral origin of replication (see, e.g., Fiers et al., Nature, 273:113 (1978); Mulligan and Berg, Science, 209:1422-1427 (1980); and Pavlakis et al., Proc. Natl. Acad. Sci. USA, 78:7398-7402 (1981)). The immediate early promoter of the human cytomegalovirus (CMV) is conveniently obtained as a Hind III E restriction fragment (see, e.g., Greenaway et al., Gene, 18:355-360 (1982)). A broad host range promoter, such as the SV40 early promoter or the Rous sarcoma virus LTR, is suitable for use in the present expression vectors.

Generally, a strong promoter may be employed to provide for high level transcription and expression of the desired product. Among the eukaryotic promoters that have been identified as strong promoters for high-level expression are the SV40 early promoter, adenovirus major late promoter, mouse metallothionein-I promoter, Rous sarcoma virus long terminal repeat, and human cytomegalovirus immediate early promoter (CMV or CMV IE). In an embodiment, the promoter is a SV40 or a CMV early promoter.

The promoters employed may be constitutive or regulatable, e.g., inducible. Exemplary inducible promoters include jun, fos and metallothionein and heat shock promoters. One or both promoters of the transcription units can be an inducible promoter. In an embodiment, the GFP is expressed from a constitutive promoter while an inducible promoter drives transcription of the gene coding for one or more enzymes as disclosed herein and/or the amplifiable selectable marker.

The transcriptional regulatory region in higher eukaryotes may comprise an enhancer sequence. Many enhancer sequences from mammalian genes are known e.g., from globin, elastase, albumin, α-fetoprotein and insulin genes. A suitable enhancer is an enhancer from a eukaryotic cell virus. Examples include the SV40 enhancer on the late side of the replication origin (bp 100-270), the enhancer of the cytomegalovirus immediate early promoter (Boshartet aL Cell 41:521 (1985)), the polyoma enhancer on the late side of the replication origin, and adenovirus enhancers (see also, e.g., Yaniv, Nature, 297:17-18 (1982) on enhancing elements for activation of eukaryotic promoters). The enhancer sequences may be introduced into the vector at a position 5′ or 3′ to the gene of interest, but is preferably located at a site 5′ to the promoter.

Yeast and mammalian expression vectors may contain prokaryotic sequences that facilitate the propagation of the vector in bacteria. Therefore, the vector may have other components such as an origin of replication (e.g., a nucleic acid sequence that enables the vector to replicate in one or more selected host cells), antibiotic resistance genes for selection in bacteria, and/or an amber stop codon which can permit translation to read through the codon. Additional eukaryotic selectable gene(s) may be incorporated. Generally, in cloning vectors the origin of replication is one that enables the vector to replicate independently of the host chromosomal DNA, and includes origins of replication or autonomously replicating sequences. Such sequences are well known, e.g., the ColE1 origin of replication in bacteria. Various viral origins (e.g., SV40, polyoma, adenovirus, VSV or BPV) are useful for cloning vectors in mammalian cells. Generally, a eukaryotic replicon is not needed for expression in mammalian cells unless extrachromosomal (episomal) replication is intended (e.g., the SV40 origin may typically be used only because it contains the early promoter).

To facilitate insertion and expression of different genes coding for the enzymes as disclosed herein from the constructs and expression vectors, the constructs may be designed with at least one cloning site for insertion of any gene coding for any enzyme disclosed herein. The cloning site may be a multiple cloning site, e.g., containing multiple restriction sites.

The plasmids may be propagated in bacterial host cells to prepare DNA stocks for subcloning steps or for introduction into eukaryotic host cells. Transfection of eukaryotic host cells can be any performed by any method well known in the art. Transfection methods include lipofection, electroporation, calcium phosphate co-precipitation, rubidium chloride or polycation mediated transfection, protoplast fusion and microinjection. Preferably, the transfection is a stable transfection. The transfection method that provides optimal transfection frequency and expression of the construct in the particular host cell line and type is favored. Suitable methods can be determined by routine procedures. For stable transfectants, the constructs are integrated so as to be stably maintained within the host chromosome.

Vectors may be introduced to selected host cells by any of a number of suitable methods known to those skilled in the art. For example, vector constructs may be introduced to appropriate cells by any of a number of transformation methods for plasmid vectors. For example, standard calcium-chloride-mediated bacterial transformation is still commonly used to introduce naked DNA to bacteria (see, e.g., Sambrook et al., 1989, Molecular Cloning, A Laboratory Manual, Cold Spring Harbor Laboratory Press, Cold Spring Harbor, N.Y.), but electroporation and conjugation may also be used (see, e.g., Ausubel et al., 1988, Current Protocols in Molecular Biology, John Wiley & Sons, Inc., NY, N.Y.).

For the introduction of vector constructs to yeast or other fungal cells, chemical transformation methods may be used (e.g., Rose et al., 1990, Methods in Yeast Genetics, Cold Spring Harbor Laboratory Press, Cold Spring Harbor, N.Y.). Transformed cells may be isolated on selective media appropriate to the selectable marker used. Alternatively, or in addition, plates or filters lifted from plates may be scanned for GFP fluorescence to identify transformed clones.

For the introduction of vectors comprising differentially expressed sequences to mammalian cells, the method used may depend upon the form of the vector. Plasmid vectors may be introduced by any of a number of transfection methods, including, for example, lipid-mediated transfection (“lipofection”), DEAE-dextran-mediated transfection, electroporation or calcium phosphate precipitation (see, e.g., Ausubel et al., 1988, Current Protocols in Molecular Biology, John Wiley & Sons, Inc., NY, N.Y.).

Lipofection reagents and methods suitable for transient transfection of a wide variety of transformed and non-transformed or primary cells are widely available, making lipofection an attractive method of introducing constructs to eukaryotic, and particularly mammalian cells in culture. For example, LipofectAMINE™ (Life Technologies) or LipoTaxi™ (Stratagene) kits are available. Other companies offering reagents and methods for lipofection include Bio-Rad Laboratories, CLONTECH, Glen Research, InVitrogen, JBL Scientific, MBIFermentas, PanVera, Promega, Quantum Biotechnologies, Sigma-Aldrich, and Wako Chemicals USA.

The host cell may be capable of expressing the construct encoding the desired protein, processing the protein and transporting a secreted protein to the cell surface for secretion. Processing includes co- and post-translational modification such as leader peptide cleavage, GPI attachment, glycosylation, ubiquitination, and disulfide bond formation. Immortalized host cell cultures amenable to transfection and in vitro cell culture and of the kind typically employed in genetic engineering are preferred. Examples of useful mammalian host cell lines are monkey kidney CV1 line transformed by SV40 (COS-7, ATCCCRL 1651); human embryonic kidney line (293 or 293 derivatives adapted for growth in suspension culture, Graham et al., J. Gen Virol., 36:59 (1977); baby hamster kidney cells (BHK, ATCC CCL 10); DHFR-Chinese hamster ovary cells (ATCC CRL-9096); dp12.CHO cells, a derivative of CHO/DHFR-(EP 307,247 published 15 Mar. 1989); mouse sertoli cells (TM4, Mather, Biol. Reprod., 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCCCRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 (1982)); PEER human acute lymphoblastic cell line (Ravid et al. Int. J. Cancer 25:705-710 (1980)); MRC 5 cells; FS4 cells; human hepatoma cell line (Hep G2), human HT1080 cells, KB cells, JW-2 cells, Detroit 6 cells, NIH-3T3 cells, hybridoma and myeloma cells. Embryonic cells used for generating transgenic animals are also suitable (e.g., zygotes and embryonic stem cells).

Suitable host cells for cloning or expressing polynucleotides (e.g., DNA) in vectors may include, for example, prokaryote, yeast, or higher eukaryote cells. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratiamarcescans, and Shigella, as well as Bacilli such as B. subtilis and B. licheniformis (e.g., B. licheniformis 41 P disclosed in DD 266,710 published Apr. 12, 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), E. coli JM110 (ATCC 47,013) and E. coli W3110 (ATCC 27,325) are suitable.

In addition to prokaryotes, eukaryotic microbes such as filamentous fungi or yeast may be suitable cloning or expression hosts for vectors comprising polynucleotides coding for one or more enzymes. Saccharomyces cerevisiae, or common baker's yeast, is the most commonly used among lower eukaryotic host microorganisms. However, a number of other genera, species, and strains are commonly available and useful herein, such as Schizosaccharomycespombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; Yarrowia (EP 402,226); Pichia pastors (EP 183,070); Candida; Trichodermareesia (EP 244,234); Neurosporacrassa; Schwanniomycessuch as Schwanniomycesoccidentalis; and filamentous fungi such as, e.g., Neurospora, Penicillium, Tolypocladium, and Aspergillus hosts such as A. nidulans and A. niger.

When the enzyme is glycosylated, suitable host cells for expression may be derived from multicellular organisms. Examples of invertebrate cells include plant and insect cells. Numerous baculoviral strains and variants and corresponding permissive insect host cells from hosts such as Spodopterafrugiperda (caterpillar), Aedesaegypti (mosquito), Aedesalbopictus (mosquito), Drosophila melanogaster (fruitfly), and Bombyxmori (silk moth) have been identified. A variety of viral strains for transfection are publicly available, e.g., the L-1 variant of Autographacalifornica NPV and the Bm-5 strain of Bombyxmori NPV, and such viruses may be used as the virus herein according to the present disclosure, particularly for transfection of Spodopterafrugiperda cells.

Plant cell cultures of cotton, corn, potato, soybean, petunia, tomato, tobacco, lemna, and other plant cells can also be utilized as host cells.

Examples of useful mammalian host cells are Chinese hamster ovary cells, including CHOK1 cells (ATCC CCL61), DXB-11, DG-44, and Chinese hamster ovary cells/−DHFR (CHO, Urlaub et al., Proc. Natl. Acad. Sci. USA 77: 4216 (1980)); monkey kidney CV1 line transformed by SV40 (COS-7, ATCCCRL 1651); human embryonic kidney line (293 or 293 cells subcloned for growth in suspension culture, (Graham et al., J. Gen Virol. 36: 59, 1977); baby hamster kidney cells (BHK, ATCC CCL 10); mouse sertoli cells (TM4, Mather, (Biol. Reprod. 23: 243-251, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCCCRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383: 44-68 (1982)); MRC 5 cells; FS4 cells; and a human hepatoma line (Hep G2).

Host cells are transformed or transfected with the above-described expression or cloning vectors for production of one or more enzymes as disclosed herein or with polynucleotides coding for one or more enzymes as disclosed herein and cultured in conventional nutrient media modified as appropriate for inducing promoters, selecting transformants, or amplifying the genes encoding the desired sequences.

Host cells containing desired nucleic acid sequences coding for the disclosed enzymes may be cultured in a variety of media. Commercially available media such as Ham's F10 (Sigma), Minimal Essential Medium ((MEM), Sigma), RPMI-1640 (Sigma), and Dulbecco's Modified Eagle's Medium ((DMEM), Sigma) are suitable for culturing the host cells. In addition, any of the media described in Ham et al., Meth. Enz. 58: 44, (1979); Barnes et al., Anal. Biochem. 102: 255 (1980); U.S. Pat. Nos. 4,767,704; 4,657,866; 4,927,762; 4,560,655; or 5,122,469; WO90103430; WO 87/00195; or U.S. Pat. Re. No. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

### Polynucleotides and Encoded Enzymes

Any known polynucleotide (e.g., gene) that codes for an enzyme or variant thereof that is capable of catalyzing an enzymatic conversion including, for example, an enzyme as set forth in Table 1 or FIG. 1, is contemplated for use by the present disclosure. Such polynucleotides may be modified (e.g., genetically engineered) to modulate (e.g., increase or decrease) the substrate specificity of an encoded enzyme, or the polynucleotides may be modified to change the substrate specificity of the encoded enzyme (e.g., a polynucleotide that codes for an enzyme with specificity for a substrate may be modified such that the enzyme has specificity for an alternative substrate). Preferred microorganisms may comprise polynucleotides coding for one or more of the enzymes as set forth in Table 1 and FIG. 1.

Enzymes and polynucleotides encoding same, for catalyzing the conversions in Table 1 and FIG. 1 are categorized in Table 2, by Enzyme Commission (EC) number, function, and the step in Table 1 and FIG. 1 in which they catalyze a conversion.

TABLE 2
Exemplary genes coding for enzymes in Table 1 and FIG. 1

| | | | | Gene ID (GI) | SEQ |
|---|---|---|---|---|---|
| Enzyme | | | | or Accession | ID |
| No. | EC No. | Gene | Organism | Number (AN) | NO: |
| A | 4.1.1.49 | pckA | Escherichia coli | 12933187 | 1 |
| A | 4.1.1.49 | pckA | Actinobacillus | 5349670 | 2 |
| | | | succinogenes | | |
| B | 1.1.1.37 | mdh1 | Escherichia coli | 12931785 | 3 |
| B | 1.1.1.37 | mdh2 | Saccharomyces | 853994 | 4 |
| | | | cerevisiae | | |
| B | 1.1.1.37 | Mqo | Escherichia coli | 12933223 | 5 |
| C | 4.2.1.2 | fum1 | Saccharomyces | 855866 | 6 |
| | | | cerevisiae | | |
| C | 4.2.1.2 | fumA | Escherichia coli | 12934128 | 7 |
| C | 4.2.1.2 | fumB | Escherichia coli | 12933156 | 8 |
| C | 4.2.1.2 | fumC | Escherichia coli | 12934129 | 9 |
| D | 1.3.99.1 | frdA | Escherichia coli | 12931889 | 10 |
| D | 1.3.99.1 | frdB | Escherichia coli | 12933707 | 11 |
| D | 1.3.99.1 | frdC | Escherichia coli | 12933706 | 12 |
| D | 1.3.99.1 | frdD | Escherichia coli | 12933705 | 13 |
| D | 1.3.5.1 | sdhA | Escherichia coli | 12933913 | 14 |
| D | 1.3.5.1 | sdhB | Escherichia coli | 12932956 | 15 |
| D | 1.3.5.1 | sdhC | Escherichia coli | 12930948 | 16 |
| D | 1.3.5.1 | sdhD | Escherichia coli | 12930949 | 17 |
| E | 2.3.1.54 | pflB | Escherichia coli | 12931841 | 18 |
| F | 1.1.99.33 | fdhF | Escherichia coli | 12933956 | 19 |
| F | 1.1.99.33 | hycD | Escherichia | 12953200 | 20 |
| | | | blattae | | |
| F | 1.1.99.33 | hycC | Escherichia | 12953199 | 21 |
| | | | blattae | | |
| F | 1.1.99.33 | hycE | Escherichia | 12953201 | 22 |
| | | | blattae | | |
| F | 1.1.99.33 | hycF | Escherichia | 12953202 | 23 |
| | | | blattae | | |
| F | 1.1.99.33 | hycG | Escherichia | 12953203 | 24 |
| | | | blattae | | |
| F | 1.1.99.33 | hycB | Escherichia | 12956669 | 25 |
| | | | blattae | | |
| G | 1.12.1.2 | hoxH | Cupriavidus | 2656817 | 26 |
| | | | necator | | |
| G | 1.12.1.2 | hoxY | Cupriavidus | 2656816 | 27 |
| | | | necator | | |
| G | 1.12.1.2 | hoxF | Cupriavidus | 2656814 | 28 |
| | | | necator | | |
| G | 1.12.1.2 | hoxU | Cupriavidus | 2656815 | 29 |
| | | | necator | | |
| H | 2.3.3.10 | erg13 | Saccharomyces | 854913 | 30 |
| | | | cerevisiae | | |
| I | 1.1.1.88 | mvaA | Staphylococcus | 2861328 | 31 |
| | | | aureus | | |
| I | 1.1.1.34 | mvaA | Staphylococcus | 3794135 | 32 |
| | | | aureus | | |
| I | 1.1.1.34 | mvaA | Saccharomyces | 854900 | 33 |
| | | | cerevisiae | | |
| J | 2.7.1.36 | erg12 | Saccharomyces | 855248 | 34 |
| | | | cerevisiae | | |
| K | 2.7.4.2 | erg8 | Saccharomyces | 855260 | 35 |
| | | | cerevisiae | | |
| L | 4.1.1.32 | mvd1 | Saccharomyces | 855779 | 36 |
| | | | cerevisiae | | |
| M | 5.3.3.2 | ldi | Escherichia coli | 12930440 | 37 |
| N | 4.2.3.27 | ispS | Populus alba | 63108309 | 38 |
| O | 2.5.1.1 | erg20 | Saccharomyces | 853272 | 39 |
| | | | cerevisiae | | |
| P | 2.5.1.10 | ispA | Escherichia coli | 12930843 | 40 |
| Q | 4.2.3.46 | afs1 | Malus | 32265057 | 41 |
| | | | domestica | | |

### Methods for the Co-Production of One or More Terpenes and Co-Products

One or more terpenes (e.g., isoprene and/or farnesene) and succinate may be produced by contacting any of the disclosed genetically modified microorganisms with a fermentable carbon source. Such methods may preferably comprise contacting a fermentable carbon source with a microorganism comprising one or more polynucleotides coding for enzymes in a pathway that catalyze the conversion of the fermentable carbon source into any of the intermediates provided in Table 1 or FIG. 1 and one or more polynucleotides coding for enzymes in a pathway that catalyze a conversion one or more of the intermediates provided in FIG. 1 (table 1) into one or more terpenes and succinate in a fermentation media; and expressing the one or more polynucleotides coding for the enzymes in the pathway that catalyzes the conversion of the fermentable carbon source into one or more of the intermediates provided in FIG. 1 (table 1) and one or more polynucleotides coding for enzymes in a pathway that catalyzes the conversion of one or more intermediates provided in FIG. 1 (table 1) into one or more terpenes and succinate. For example, the microorganism may be modified to comprise one or more polynucleotides coding for enzymes that catalyze a conversion of one or more intermediates of the mevalonate pathway to one or more terpenes such as isoprene and/or farnesene. Optionally, the microorganism may be further modified to comprise one or more polynucleotides coding for enzymes that catalyze a conversion of an intermediate of Table 1 such as oxaloacetate to succinate.

The metabolic pathways that lead to the production of industrially important compounds involve oxidation-reduction (redox) reactions. For example, during fermentation, glucose is oxidized in a series of enzymatic reactions into smaller molecules with the concomitant release of energy. The electrons released are transferred from one reaction to another through universal electron carriers, such Nicotinamide Adenine Dinucleotide (NAD) and Nicotinamide Adenine Dinucleotide Phosphate (NAD(P)), which act as cofactors for oxidoreductase enzymes. In microbial catabolism, glucose is oxidized by enzymes using the oxidized form of the cofactors (NAD(P)+ and/or NAD+) as cofactor thus generating reducing equivalents in the form of the reduced cofactor (NAD(P)H and NADH). In order for fermentation to continue, redox-balanced metabolism is required, i.e., the cofactors must be regenerated by the reduction of microbial cell metabolic compounds.

Microorganism-catalyzed fermentation for the production of natural products is a widely known application of biocatalysis. Industrial microorganisms can affect multistep conversions of renewable feedstocks to high value chemical products in a single reactor. Products of microorganism-catalyzed fermentation processes range from chemicals such as ethanol, lactic acid, amino acids and vitamins, to high value small molecule pharmaceuticals, protein pharmaceuticals, and industrial enzymes. In many of these processes, the biocatalysts are whole-cell microorganisms, including microorganisms that have been genetically modified to express heterologous genes.

Some key parameters for efficient microorganism-catalyzed fermentation processes include the ability to grow microorganisms to a greater cell density, increased yield of desired products, increased amount of volumetric productivity, removal of unwanted co-metabolites, improved utilization of inexpensive carbon and nitrogen sources, adaptation to varying fermenter conditions, increased production of a primary metabolite, increased production of a secondary metabolite, increased tolerance to acidic conditions, increased tolerance to basic conditions, increased tolerance to organic solvents, increased tolerance to high salt conditions and increased tolerance to high or low temperatures. Inefficiencies in any of these parameters can result in high manufacturing costs, inability to capture or maintain market share, and/or failure to bring fermented end-products to market.

The methods and compositions of the present disclosure can be adapted to conventional fermentation bioreactors (e.g., batch, fed-batch, cell recycle, and continuous fermentation).

In some embodiments, a microorganism (e.g., a genetically modified microorganism) as provided herein is cultivated in liquid fermentation media (i.e., a submerged culture) which leads to excretion of the fermented product(s) into the fermentation media. In one embodiment, the fermented end product(s) can be isolated from the fermentation media using any suitable method known in the art.

In some embodiments, formation of the fermented product occurs during an initial, fast growth period of the microorganism. In one embodiment, formation of the fermented product occurs during a second period in which the culture is maintained in a slow-growing or quiescent state. In one embodiment, formation of the fermented product occurs during more than one growth period of the microorganism. In such embodiments, the amount of fermented product formed per unit of time is generally a function of the metabolic activity of the microorganism, the physiological culture conditions (e.g., pH, temperature, medium composition), and the amount of microorganisms present in the fermentation process.

In some embodiments, the fermentation product is recovered from the periplasm or culture medium as a secreted metabolite. In one embodiment, the fermentation product is extracted from the microorganism, for example when the microorganism lacks a secretory signal corresponding to the fermentation product. In one embodiment, the microorganisms are ruptured and the culture medium or lysate is centrifuged to remove particulate cell debris. The membrane and soluble protein fractions may then be separated if necessary. The fermentation product of interest may then be purified from the remaining supernatant solution or suspension by, for example, distillation, fractionation, chromatography, precipitation, filtration, and the like. In one embodiment, the microorganism cells (or portions thereof) may be used as biocatalysts or for other functions in a subsequent process without substantial purification.

Several types of metabolic conversions involve redox reactions. Electron transfer usually requires participation of redox cofactors such as NADH, NADPH and ferredoxin. Amounts of cofactors in the cell are limited: typical concentrations of NADH are 20-70 μmol/g cell dry weight (CDW) in Escherichia coli and 3.4 μmol/g CDW in Saccharomyces cerevisiae. To allow the continuation of metabolic processes, cofactors may have to be regenerated. Oxygen plays an important role in cofactor regeneration because it is the terminal acceptor of electrons in the electron transfer system. Three different modes of cofactor utilization can be distinguished with respect to the conversion of substrates into products: (i) conversions that do not result in net cofactor oxidation or reduction; (ii) conversions that results in cofactor reduction; and (iii) conversions that result in cofactor oxidation. The latter two require additional pathways to regenerate the cofactors, which strongly influence the overall yield and productivity.

The degree of reduction of a product as well as the ATP requirement of its synthesis determines whether a production process is able to proceed aerobically or anaerobically. To produce the products disclosed in FIG. 1 via anaerobic microbial conversion, or at least by using a process with reduced oxygen consumption, redox imbalances must be avoided. As discussed below, alternative ways of cofactor regeneration can be engineered, and in some cases, additional sources of ATP may have to be provided. Another option is to separate oxidizing and reducing processes spatially in bioelectrochemical systems.

In the methods of the present disclosure, if the utilization of sugar results in a deficit or surplus of electrons, the oxygen requirement can be circumvented by using more reduced or oxidized substrates, respectively. For example, galacturonic acid—the major constituent of pectin, abundantly available in agricultural side streams.—is more oxidized than glucose, with a difference of two electron pairs (Grohmann, K. et al. (1998) Biotechnol. Lett. 20, 195-200). Galacturonic acid is a suitable substrate for the production of oxidized products, such citric acid. Compared to glucose, conversion of galacturonic acid into citric acid results in a threefold decrease in NADH production, and thus a threefold lower oxygen requirement.

Glycerol is a major byproduct of biodiesel production, and is an attractive fermentation substrate due to its low price (Yazdani, S. S. and Gonzalez, R. (2007) Curr. Opin. Biotechnol. 18, 213-219) and is contemplated as a fermentable substrate in the methods of the present disclosure. It is more reduced than sugars, and, therefore, suitable for the synthesis of compounds, such as succinic acid, which production from sugar results in cofactor oxidation. This approach has been used for the anaerobic production of succinic acid. If the conversion reaction results in electron deficit, co-substrates can be added that function as electron donors (Babel, W. (2009) Eng. Life Sci. 9, 285-290).

Besides using a redox-neutral substrate or substrate mixture to avoid oxygen requirement, it is possible to convert the substrate into a mixture of products. E. coli, for example, converts sugars under anaerobic conditions into a redox-neutral mixture of products including acetate, ethanol, succinate, lactate, formate, CO₂ and/or hydrogen gas; some of which result in net cofactor oxidation or reduction (Clark, D. P. (1989) Micobiol. Rev. 63, 223-234). The bulk chemical succinic acid, whose formation results in cofactor oxidation, can therefore be produced under anaerobic conditions. Nevertheless, using a synthetic biology approach, it is possible to combine the production of succinate with that of another valuable compound whose formation results in cofactor reduction, and knock out routes to other byproducts. Such approach has been used to compare maximum theoretical yields of L-lysine production, which results in cofactor oxidation, and L-glutamic acid, which results in cofactor reduction, as the only products under aerobic conditions, with the simultaneous redox neutral production of both compounds. The L-lysine yield increases from 0.41 mol C/mol C, as the only product under aerobic conditions, to 0.58 mol C/mol C in the combined process. The total product yield on glucose (L-lysine and L-glutamate) increased to 0.89 mol C/mol C. By generating both products simultaneously, the product yield on oxygen for L-lysine and L-glutamate (1.97 and 2.97 mol C/mol O₂, respectively) decreases to zero, which indicates that the process is not limited by oxygen availability. This model calculation shows that costs for raw materials and capital during most aerobic bioconversions can be reduced significantly. The risk of such approach is that the costs for product recovery might increase; however, cost reduction can be achieved by choosing a product pair that can be easily separated from each other. In that sense, this disclosure provides a method to avoid the separation drawbacks of co-production by choosing a pair of coupled pathways that result in an anaerobic process and the products are physically separated in the off-reactor streams: a gaseous terpene (e.g. isoprene) upper stream and liquid co-product stream that will have different downstream processes, or a water immiscible liquid terpene stream and a water soluble co-product stream that will have different downstream processes.

Methods for the Production of Polyisoprene and Other Compounds from Isoprene and their Applications

Host cells are cultivated in a bioreactor and the isoprene produced by cells vaporizes and forms a gaseous isoprene composition. At room temperature or under fermentative conditions (20-45° C.), isoprene gas can accumulate in the headspace of a fermentation tank. Gaseous isoprene can be siphoned and concentrated. Gaseous Isoprene has poor solubility in the aqueous phase of the fermentation broth, and poses little or no toxicity to the producing organisms or contaminants. Isoprene can be purified from fermentation of gases, including gaseous alcohol, CO₂ and other compounds by solvent extraction, cryogenic processes, distillation, fractionation, chromatography, precipitation, filtration, and the like.

Isoprene produced via any of the disclosed processes or methods may be converted to polyisoprene, lattices of polyisoprene, Styrene-Isoprene-Styrene (SIS) Block Copolymer and styrene/isoprene/butadiene rubber (SIBR). Those polymers applications include productions of tires, mechanical goods, footwear, healthcare and adhesives.

### Methods for the Production of Succinate and its Applications

After fermentation, succinate can be separated from microbial cells by centrifugation. In further downstream processes, succinate can be purified from broth by distillation, use of adsorption columns, liquid-liquid extraction and/or esterification and distillation (reactive distillation).

Succinate is a moderately high value chemical. It is a key compound to produce more than 30 commercially important products such as tetrahydrofuran (THF), 1,4-butanediol, succindiamide, succinonitrile, dimethylsuccinate, N-methyl-pyrrolidone, 2-pyrrolidone, and 1,4-diaminobutane. It has applications in industries such as food, pharmaceutical, polymers, paints, cosmetics, and inks. It is also used as a surfactant, detergent extender, antifoam, and ion-chelator.

### EXAMPLES

### Example 1

### Modification of Microorganism for Co-Production of One or More Terpenes and Succinate

A microorganism such as a bacterium is genetically modified to co-produce a terpene (e.g. isoprene, farnesene and/or squalene) and succinate from a fermentable carbon source including, for example, glucose.

In an exemplary method, a microorganism is genetically engineered by any methods known in the art to comprise one or more enzymes in a pathway that catalyze a conversion of the fermentable carbon source to one or more intermediates in a pathway for production of terpene, succinate, and hydrogen; and one or more polynucleotides in a microorganism that encode one or more enzymes in a pathway that catalyze a conversion of one or more intermediates to terpene, succinate, and hydrogen, wherein the one or more intermediates in the pathway for the production of terpene include one or more intermediates in a mevalonate pathway, and wherein the one or more intermediates in the pathway for the production of succinate include oxaloacetate.

### Example 2

### Fermentation of Glucose by Genetically Modified Microorganism to Produce One or More Terpenes, Succinate and Hydrogen

A genetically modified microorganism, as produced in Example 1 above, may be used to ferment a carbon source producing a terpene (e.g. isoprene), succinate, and hydrogen.

In an exemplary method, a previously-sterilized culture medium comprising a fermentable carbon source (e.g., 9 g/L glucose, 1 g/L KH₂PO₄, 2 g/L (NH₄)₂HPO₄, 5 mg/L FeSO₄.7H₂O, 10 mg/L MgSO₄.7H₂O, 2.5 mg/L MnSO₄.H₂O, 10 mg/L CaCl₂.6H₂O, 10 mg/L CoCl₂.6H₂O, and 10 g/L yeast extract) is charged in a bioreactor.

During fermentation, anaerobic conditions are maintained by, for example, sparging nitrogen through the culture medium. A suitable temperature for fermentation (e.g., about 30° C.) is maintained using any method known in the art. A near physiological pH (e.g., about 6.5) is maintained by, for example, automatic addition of sodium hydroxide. The bioreactor is agitated at, for example, about 50 rpm. Fermentation is allowed to run to completion.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein can be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary, only, with the true scope and spirit of the disclosure being indicated by the following claims. All references, patents, and patent applications referred to in this application are herein incorporated by reference in their entirety.

What is claimed is:

1. A method of co-producing a terpene, succinate, and hydrogen from a fermentable carbon source comprising:
a.) providing a fermentable carbon source;
b.) expressing one or more exogenous polynucleotides in a microorganism that encode one or more enzymes in a pathway that catalyze a conversion of the fermentable carbon source to one or more intermediates in a pathway for production of terpene, succinate, and hydrogen;
c.) expressing one or more polynucleotides in a microorganism that encode one or more enzymes in a pathway that catalyze a conversion of one or more intermediates to terpene, succinate, and hydrogen; and
d.) contacting the fermentable carbon source with the microorganism,
wherein the one or more intermediates in the pathway for the production of terpene include one or more intermediates in a mevalonate pathway, wherein the one or more intermediates in the pathway for the production of succinate include oxaloacetate, and wherein the co-production method is anaerobic.

2. The method of claim 1, wherein the terpene is isoprene, farnesene, squalene, and/or bisabolene.

3. The method of claim 1, wherein the one or more enzymes that catalyze the conversion of the fermentable carbon source to one or more intermediates in the pathway for the production of terpene, succinate, and hydrogen are set forth in Table 1.

4. The method of claim 1, wherein the one or more enzymes that catalyze the conversion of the one or more intermediates to terpene, succinate, and hydrogen are set forth in Table 1.

5. The method of claim 1, wherein the microorganism is a bacteria selected from the genera consisting essentially of: Propionibacterium, Propionispira, Clostridium, Bacillus, Escherichia, Pelobacter, or Lactobacillus.

6. The method of claim 1, wherein the microorganism is a eukaryote selected from the group consisting essentially of a yeast, filamentous fungi, protozoa, or algae.

7. The method of claim 1, wherein the microorganism is from a genus selected from the group consisting of: Saccharomyces, Yarrowia, Hansenula, Pichia, Ashbya, and Candida.

8. The method of claim 1, wherein the fermentable carbon source is comprises sugarcane juice, sugarcane molasses, hydrolyzed starch, hydrolyzed lignocellulosic materials, glucose, sucrose, fructose, lactate, lactose, xylose, pyruvate, or glycerol in any form or mixture thereof.

9. The method of claim 1, wherein the fermentable carbon source is a monosaccharide, oligosaccharide, or polysaccharide.

10. The method of claim 1, wherein terpene, succinate, and hydrogen are secreted by the microorganism into the fermentation media.

11. The method of claim 10, comprising recovering terpene, succinate, and hydrogen from the fermentation media.

12. The method of claim 1, wherein the fermentable carbon source is contacted with the microorganism prior to expressing in the microorganism the one or more polynucleotides that encode one or more enzymes in a pathway that catalyze a conversion of the fermentable carbon source to one or more intermediates in a pathway for the production of isoprene, succinate, and hydrogen and the one or more polynucleotides that encode one or more enzymes in a pathway that catalyze a conversion of the one or more intermediates to isoprene, succinate, and hydrogen.

13. The method of claim 1, wherein the fermentable carbon source is contacted with the microorganism after expressing in the microorganism the one or more polynucleotides that encode one or more enzymes in a pathway that catalyze a conversion of the fermentable carbon source to one or more intermediates in a pathway for the production of isoprene, succinate, and hydrogen and the one or more polynucleotides that encode one or more enzymes in a pathway that catalyze a conversion of the one or more intermediates to isoprene, succinate, and hydrogen.

14. A microorganism comprising one or more polynucleotides that encode one or more enzymes in a pathway that catalyze a conversion of the fermentable carbon source to one or more intermediates in a pathway for the production of isoprene, succinate, and hydrogen and one or more polynucleotides that encode one or more enzymes in a pathway that catalyze a conversion of the one or more intermediates to isoprene, succinate, and hydrogen, wherein the one or more intermediates in the pathway for the production of terpene include one or more intermediates in a mevalonate pathway, and wherein the one or more intermediates in the pathway for the production of succinate include oxaloacetate.

15. The microorganism of claim 14, wherein the one or more enzymes that catalyze the conversion of the fermentable carbon source to one or more intermediates in the pathway for the production of terpene, succinate, and hydrogen are set forth in Table 1.

16. The microorganism of claim 14, wherein the one or more enzymes that catalyze the conversion of the one or more intermediates to terpene, succinate, and hydrogen are set forth in Table 1.

17. The microorganism of claim 14, wherein the microorganism is a bacteria selected from the genera consisting of: Burkholderia, Propionibacterium, Propionispira, Clostridium, Bacillus, Escherichia, Pelobacter, or Lactobacillus.

18. The microorganism of claim 14, wherein the microorganism is a eukaryote selected from the group consisting of yeast, filamentous fungi, protozoa, and algae.

19. The microorganism of claim 18, wherein the yeast is Saccharomyces cerevisiae, Zymomonas mobilis, or Pichia pastoris.

20. The microorganism of claim 14, wherein the microorganism has been genetically modified to express the one or more polynucleotides coding for enzymes in a pathway that catalyze a conversion of the fermentable carbon source to one or more intermediates in a pathway for the production of isoprene, succinate, and hydrogen and one or more polynucleotides coding for enzymes in a pathway that catalyze a conversion of the one or more intermediates to isoprene, succinate, and hydrogen.

21. A method of co-producing farnesene, succinate, and hydrogen from a glucose source comprising:
a.) providing a glucose source;
b.) expressing one or more exogenous polynucleotides in a microorganism that encode one or more enzymes in a pathway that catalyze a conversion of glucose to phosphoenolpyruvate, one or more polynucleotides coding for enzymes in a pathway that catalyze a conversion of phosphoenolpyruvate to oxaloacetate, one or more polynucleotides coding for enzymes in a pathway that catalyze a conversion of oxaloacetate to malate, one or more polynucleotides coding for enzymes in a pathway that catalyze a conversion of malate to fumarate, one or more polynucleotides coding for enzymes in a pathway that catalyze a conversion of fumarate to succinate, one or more polynucleotides coding for enzymes in a pathway that catalyze a conversion of pyruvate to acetyl-CoA and formate, one or more polynucleotides coding for enzymes in a pathway that catalyze a conversion of formate to CO₂ or formate to CO₂ and hydrogen, one or more polynucleotides coding for enzymes in a pathway that catalyze a conversion of NAD(P)H to NAD(P)⁺ and hydrogen, or a conversion of a reduced acceptor to CO₂ and hydrogen, one or more polynucleotides coding for enzymes in a pathway that catalyze a conversion of acetoacetyl-CoA and acetyl-CoA to 3-hydroxy-3-methylglutaryl-CoA, one or more polynucleotides coding for enzymes in a pathway that catalyze a conversion of 3-hydroxy-3-methylglutaryl-CoA to mevalonate, one or more polynucleotides coding for enzymes in a pathway that catalyze a conversion of mevalonate to phosphomevalonate, one or more polynucleotides coding for enzymes in a pathway that catalyze a conversion of phosphomevalonate to diphosphomevalonate, one or more polynucleotides coding for enzymes in a pathway that catalyze a conversion of diphosphomevalonate to isopentenyl diphosphate, one or more polynucleotides coding for enzymes in a pathway that catalyze a conversion of isopentenyl diphosphate to dimethylallyl diphosphate, one or more polynucleotides coding for enzymes in a pathway that catalyze a conversion of dimethylallyl diphosphate to isoprene, one or more polynucleotides coding for enzymes in a pathway that catalyze a conversion of dimethylallyl diphosphate and isopentenyl diphosphate to diphosphate and geranyl diphosphate, one or more polynucleotides coding for enzymes in a pathway that catalyze a conversion of geranyl diphosphate and isopentenyl diphosphate to diphosphate and farnesyl diphosphate, and one or more polynucleotides coding for enzymes in a pathway that catalyze a conversion of farnesyl diphosphate to farnesene; and
c.) contacting the glucose source with the microorganism,
wherein the co-production method is anaerobic.

22. The method of claim 21, wherein the enzymes in the pathway that catalyze a conversion of phosphoenolpyruvate to oxaloacetate include a PEP carboxykinase, wherein the enzymes in the pathway that catalyze a conversion of oxaloacetate to malate include a malate dehydrogenase, wherein the enzymes in the pathway that catalyze a conversion of malate to fumarate include a fumarate hydratase; wherein the enzymes in the pathway that catalyze a conversion of fumarate and a reduced acceptor to succinate and an acceptor include a fumarate dehydrogenase or a succinate dehydrogenase, wherein the enzymes in the pathway that catalyze a conversion of pyruvate to acetyl-CoA and formate include a pyruvate formate lyase; wherein the enzymes in the pathway that catalyze a conversion of formate to CO₂, or formate to CO₂ and H₂ include a formate dehydrogenase or a formate hydrogen lyase; wherein the enzymes in the pathway that catalyze a conversion of NAD(P)H to NAD(P)⁺ and hydrogen, or a conversion of a reduced acceptor to CO₂ and hydrogen include a hydrogen dehydrogenase or a hydrogen dehydrogenase (NADP+); wherein the enzymes in the pathway that catalyze a conversion of acetoacetyl-CoA and acetyl-CoA to 3-hydroxy-3-methylglutaryl-CoA include a hydroxymethylglutaryl-CoA synthase, wherein the one or more polynucleotides coding for enzymes in the pathway that catalyze a conversion of 3-hydroxy-3-methylglutaryl-CoA to mevalonate include a hydroxymethylglutaryl-CoA reductase; wherein the enzymes in the pathway that catalyze a conversion of mevalonate to phosphomevalonate include a mevalonate kinase; wherein the enzymes in the pathway that catalyze a conversion of phosphomevalonate to diphosphomevalonate include a phosphomevalonate kinase; wherein the enzymes in the pathway that catalyze a conversion of diphosphomevalonate to isopentenyl diphosphate include a diphosphomevalonate decarboxylase; wherein the enzymes in the pathway that catalyze a conversion of isopentenyl diphosphate to dimethylallyl diphosphate include an isopentenyl diphosphate delta-isomerase; wherein the enzymes in the pathway that catalyze a conversion of dimethylallyl diphosphate to isoprene include an isoprene synthase; wherein the enzymes in the pathway that catalyze a conversion of dimethylallyl diphosphate and isopentenyl diphosphate to diphosphate and geranyl diphosphate include a geranyl-diphosphate synthase; wherein the enzymes in the pathway that catalyze a conversion of geranyl diphosphate and isopentenyl diphosphate to diphosphate and farnesyl diphosphate include a farnesyl pyrophosphate synthase; and wherein the enzymes in the pathway that catalyze a conversion of farnesyl diphosphate to farnesene include a farnesene synthase.

23. The method of claim 21, wherein the microorganism is a bacteria selected from the genera consisting essentially of: Propionibacterium, Propionispira, Clostridium, Bacillus, Escherichia, Pelobacter, or Lactobacillus.

24. The method of claim 21, wherein the microorganism is a eukaryote selected from the group consisting essentially of a yeast, filamentous fungi, protozoa, or algae.

25. The method of claim 21, wherein the microorganism is from a genus selected from the group consisting of: Saccharomyces, Yarrowia, Hansenula, Pichia, Ashbya, and Candida.

26. The method of claim 21, wherein farnesene, succinate, and hydrogen are secreted by the microorganism into the fermentation media.

27. The method of claim 26, comprising recovering farnesene, succinate, and hydrogen from the fermentation media.

28. The method of claim 21, wherein the glucose source is contacted with the microorganism prior to expressing in the microorganism the one or more exogenous polynucleotides in a microorganism that encode one or more enzymes in a pathway that catalyze a conversion of glucose to phosphoenolpyruvate, one or more polynucleotides coding for enzymes in a pathway that catalyze a conversion of phosphoenolpyruvate to oxaloacetate, one or more polynucleotides coding for enzymes in a pathway that catalyze a conversion of oxaloacetate to malate, one or more polynucleotides coding for enzymes in a pathway that catalyze a conversion of malate to fumarate, one or more polynucleotides coding for enzymes in a pathway that catalyze a conversion of fumarate to succinate, one or more polynucleotides coding for enzymes in a pathway that catalyze a conversion of pyruvate to acetyl-CoA and formate, one or more polynucleotides coding for enzymes in a pathway that catalyze a conversion of formate to CO₂ or formate to CO₂ and hydrogen, one or more polynucleotides coding for enzymes in a pathway that catalyze a conversion of NAD(P)H to NAD(P)⁺ and hydrogen, or a conversion of a reduced acceptor to CO₂ and hydrogen, one or more polynucleotides coding for enzymes in a pathway that catalyze a conversion of acetoacetyl-CoA and acetyl-CoA to 3-hydroxy-3-methylglutaryl-CoA, one or more polynucleotides coding for enzymes in a pathway that catalyze a conversion of 3-hydroxy-3-methylglutaryl-CoA to mevalonate, one or more polynucleotides coding for enzymes in a pathway that catalyze a conversion of mevalonate to phosphomevalonate, one or more polynucleotides coding for enzymes in a pathway that catalyze a conversion of phosphomevalonate to diphosphomevalonate, one or more polynucleotides coding for enzymes in a pathway that catalyze a conversion of diphosphomevalonate to isopentenyl diphosphate, one or more polynucleotides coding for enzymes in a pathway that catalyze a conversion of isopentenyl diphosphate to dimethylallyl diphosphate, one or more polynucleotides coding for enzymes in a pathway that catalyze a conversion of dimethylallyl diphosphate to isoprene, one or more polynucleotides coding for enzymes in a pathway that catalyze a conversion of dimethylallyl diphosphate and isopentenyl diphosphate to diphosphate and geranyl diphosphate, one or more polynucleotides coding for enzymes in a pathway that catalyze a conversion of geranyl diphosphate and isopentenyl diphosphate to diphosphate and farnesyl diphosphate, and one or more polynucleotides coding for enzymes in a pathway that catalyze a conversion of farnesyl diphosphate to farnesene

29. The method of claim 21, wherein the glucose source is contacted with the microorganism after expressing in the microorganism the one or more exogenous polynucleotides in a microorganism that encode one or more enzymes in a pathway that catalyze a conversion of glucose to phosphoenolpyruvate, one or more polynucleotides coding for enzymes in a pathway that catalyze a conversion of phosphoenolpyruvate to oxaloacetate, one or more polynucleotides coding for enzymes in a pathway that catalyze a conversion of oxaloacetate to malate, one or more polynucleotides coding for enzymes in a pathway that catalyze a conversion of malate to fumarate, one or more polynucleotides coding for enzymes in a pathway that catalyze a conversion of fumarate to succinate, one or more polynucleotides coding for enzymes in a pathway that catalyze a conversion of pyruvate to acetyl-CoA and formate, one or more polynucleotides coding for enzymes in a pathway that catalyze a conversion of formate to CO₂ or formate to CO₂ and hydrogen, one or more polynucleotides coding for enzymes in a pathway that catalyze a conversion of NAD(P)H to NAD(P)⁺ and hydrogen, or a conversion of a reduced acceptor to CO₂ and hydrogen, one or more polynucleotides coding for enzymes in a pathway that catalyze a conversion of acetoacetyl-CoA and acetyl-CoA to 3-hydroxy-3-methylglutaryl-CoA, one or more polynucleotides coding for enzymes in a pathway that catalyze a conversion of 3-hydroxy-3-methylglutaryl-CoA to mevalonate, one or more polynucleotides coding for enzymes in a pathway that catalyze a conversion of mevalonate to phosphomevalonate, one or more polynucleotides coding for enzymes in a pathway that catalyze a conversion of phosphomevalonate to diphosphomevalonate, one or more polynucleotides coding for enzymes in a pathway that catalyze a conversion of diphosphomevalonate to isopentenyl diphosphate, one or more polynucleotides coding for enzymes in a pathway that catalyze a conversion of isopentenyl diphosphate to dimethylallyl diphosphate, one or more polynucleotides coding for enzymes in a pathway that catalyze a conversion of dimethylallyl diphosphate to isoprene, one or more polynucleotides coding for enzymes in a pathway that catalyze a conversion of dimethylallyl diphosphate and isopentenyl diphosphate to diphosphate and geranyl diphosphate, one or more polynucleotides coding for enzymes in a pathway that catalyze a conversion of geranyl diphosphate and isopentenyl diphosphate to diphosphate and farnesyl diphosphate, and one or more polynucleotides coding for enzymes in a pathway that catalyze a conversion of farnesyl diphosphate to farnesene.

30. A microorganism comprising one or more polynucleotides coding for enzymes in a pathway that catalyze a conversion of phosphoenolpyruvate to oxaloacetate, one or more polynucleotides coding for enzymes in a pathway that catalyze a conversion of oxaloacetate to malate, one or more polynucleotides coding for enzymes in a pathway that catalyze a conversion of malate to fumarate, one or more polynucleotides coding for enzymes in a pathway that catalyze a conversion of fumarate to succinate, one or more polynucleotides coding for enzymes in a pathway that catalyze a conversion of pyruvate to acetyl-CoA and formate, one or more polynucleotides coding for enzymes in a pathway that catalyze a conversion of formate to CO₂ or formate to CO₂ and hydrogen, one or more polynucleotides coding for enzymes in a pathway that catalyze a conversion of NAD(P)H to NAD(P)⁺ and hydrogen, or a conversion of a reduced acceptor to CO₂ and hydrogen, one or more polynucleotides coding for enzymes in a pathway that catalyze a conversion of acetoacetyl-CoA and acetyl-CoA to 3-hydroxy-3-methylglutaryl-CoA, one or more polynucleotides coding for enzymes in a pathway that catalyze a conversion of 3-hydroxy-3-methylglutaryl-CoA to mevalonate, one or more polynucleotides coding for enzymes in a pathway that catalyze a conversion of mevalonate to phosphomevalonate, one or more polynucleotides coding for enzymes in a pathway that catalyze a conversion of phosphomevalonate to diphosphomevalonate, one or more polynucleotides coding for enzymes in a pathway that catalyze a conversion of diphosphomevalonate to isopentenyl diphosphate, one or more polynucleotides coding for enzymes in a pathway that catalyze a conversion of isopentenyl diphosphate to dimethylallyl diphosphate, one or more polynucleotides coding for enzymes in a pathway that catalyze a conversion of dimethylallyl diphosphate to isoprene, one or more polynucleotides coding for enzymes in a pathway that catalyze a conversion of dimethylallyl diphosphate and isopentenyl diphosphate to diphosphate and geranyl diphosphate, one or more polynucleotides coding for enzymes in a pathway that catalyze a conversion of geranyl diphosphate and isopentenyl diphosphate to diphosphate and farnesyl diphosphate, and one or more polynucleotides coding for enzymes in a pathway that catalyze a conversion of farnesyl diphosphate to farnesene.

31. The method of claim 30, wherein the microorganism is a bacteria selected from the genera consisting essentially of: Propionibacterium, Propionispira, Clostridium, Bacillus, Escherichia, Pelobacter, or Lactobacillus.

32. The method of claim 30, wherein the microorganism is a eukaryote selected from the group consisting essentially of a yeast, filamentous fungi, protozoa, or algae.

33. The method of claim 30, wherein the microorganism is from a genus selected from the group consisting of: Saccharomyces, Yarrowia, Hansenula, Pichia, Ashbya, and Candida.


SEQUENCE LISTING (41 sequences)

SEQ ID NO: 1 - DNA, 2109 bp, Artificial sequence
  Synthesized pckA
aggaatgcgattccactcacaatattcccgccatataaaccaagatttaaccttttgagaacattttccacacctaaaatgctatttctgcgataatagcaaccgtttcgtgacaggaatcacggagttttttgtcaaatatgaatttctccagatacgtaaatctatgagccttgtcgcggttaacacccccaaaaagactttactattcaggcaatacatattggctaaggagcagtgaaatgcgcgttaacaatggtttgaccccgcaagaactcgaggcttatggtatcagtgacgtacatgatatcgtttacaacccaagctacgacctgctgtatcaggaagagctcgatccgagcctgacaggttatgagcgcggggtgttaactaatctgggtgccgttgccgtcgataccgggatcttcaccggtcgttcaccaaaagataagtatatcgtccgtgacgataccactcgcgatactttctggtgggcagacaaaggcaaaggtaagaacgacaacaaacctctctctccggaaacctggcagcatctgaaaggcctggtgaccaggcagctttccggcaaacgtctgttcgttgtcgacgctttctgtggtgcgaacccggatactcgtctttccgtccgtttcatcaccgaagtggcctggcaggcgcattttgtcaaaaacatgtttattcgcccgagcgatgaagaactggcaggtttcaaaccagactttatcgttatgaacggcgcgaagtgcactaacccgcagtggaaagaacagggtctcaactccgaaaacttcgtggcgtttaacctgaccgagcgcatgcagctgattggcggcacctggtacggcggcgaaatgaagaaagggatgttctcgatgatgaactacctgctgccgctgaaaggtatcgcttctatgcactgctccgccaacgttggtgagaaaggcgatgttgcggtgttcttcggcctttccggcaccggtaaaaccaccctttccaccgacccgaaacgtcgcctgattggcgatgacgaacacggctgggacgatgacggcgtgtttaacttcgaaggcggctgctacgcaaaaactatcaagctgtcgaaagaagcggaacctgaaatctacaacgctatccgtcgtgatgcgttgctggaaaacgtcaccgtgcgtgaagatggcactatcgactttgatgatggttcaaaaaccgagaacacccgcgtttcttatccgatctatcacatcgataacattgttaagccggtttccaaagcgggccacgcgactaaggttatcttcctgactgctgatgctttcggcgtgttgccgccggtttctcgcctgactgccgatcaaacccagtatcacttcctctctggcttcaccgccaaactggccggtactgagcgtggcatcaccgaaccgacgccaaccttctccgcttgcttcggcgcggcattcctgtcgctgcacccgactcagtacgcagaagtgctggtgaaacgtatgcaggcggcgggcgcgcaggcttatctggttaacactggctggaacggcactggcaaacgtatctcgattaaagatacccgcgccattatcgacgccatcctcaacggttcgctggataatgcagaaaccttcactctgccgatgtttaacctggcgatcccaaccgaactgccgggcgtagacacgaagattctcgatccgcgtaacacctacgcttctccggaacagtggcaggaaaaagccgaaaccctggcgaaactgtttatcgacaacttcgataaatacaccgacacccctgcgggtgccgcgctggtagcggctggtccgaaactgtaatgatttgaagctggagaatatctatccagtatcttatagaaagcaaaacgggaggcaccttcgcctcccgtttatttacccttcttttgtcgtgccctgcgcccgcgttaccggcactggcagccaggcgcgaatggaaagcccgccccgctcgctggtgccaagctccagcatcccgttatggttatccacgatacgctgcacaattgccagccctaatcccgtgccgctaatggtgcgcgca

SEQ ID NO: 2 - DNA, 2101 bp, Artificial sequence
  Synthesized pckA
gacgctatcgataaattgaaaatgcagcaatagaggaaacacggtttgtttgagtgaaaacagccgtgttttttcatttaccgccataaaaatttgaaacggatcacaaatcatgaaaaaaatacgttcaaattagaactaattatcgaaaatttgatctagttaacattttttaggtataaatagttttaaaatagatctagtttggatttttaattttaaattatcaatgaggtgaagtatgactgacttaaacaaactcgttaaagaacttaatgacttagggcttaccgatgttaaggaaattgtgtataacccgagttatgaacaacttttcgaggaagaaaccaaaccgggtttggagggtttcgataaagggacgttaaccacgcttggcgcggttgccgtcgatacggggatttttaccggtcgttcaccgaaagataaatatatcgtttgcgatgaaactacgaaagacaccgtttggtggaacagcgaagcggcgaaaaacgataacaaaccgatgacgcaagaaacttggaaaagtttgagagaattagtggcgaaacaactttccggtaaacgtttattcgtggtagaaggttactgcggcgccagtgaaaaacaccgtatcggtgtgcgtatggttactgaagtggcatggcaggcgcattttgtgaaaaacatgtttatccgaccgaccgatgaagagttgaaaaatttcaaagcggattttaccgtgttaaacggtgctaaatgtactaatccgaactggaaagaacaaggtttgaacagtgaaaactttgtcgctttcaatattaccgaaggtattcagcttatcggcggtacttggtacggcggtgaaatgaaaaaaggtatgttctcaatgatgaactacttcctgccgttaaaaggtgtggcttccatgcactgttccgccaacgtaggtaaagacggtgacgtggctattttcttcggtttatccggtacgggtaaaacaacgctttcgaccgatcctaaacgccaattaatcggtgatgacgaacacggttgggatgaatccggcgtatttaactttgaaggcggttgttacgcgaaaaccattaacttatctcaagaaaacgaaccggatatttacggcgcaatccgtcgtgacgcattattagaaaacgtcgtggttcgtgcagacggttccgttgactttgacgacggttcaaaaacagaaaatacccgtgtttcatatccgatttaccacatcgacaacatcgttcgtccggtatcgaaagccggtcatgcaaccaaagtgattttcttaaccgcggacgcattcggcgtattgccgccggtttcaaaactgactccggaacaaaccgaatactacttcttatccggctttactgcaaaattagcgggtacggaacgcggcgtaaccgaaccgactccgacattctcggcctgtttcggtgcggcattcttaagcctgcatccgattcaatatgcggacgtgttggtcgaacgcatgaaagcctccggtgcggaagcttatttggtgaacaccggttggaacggcacgggtaaacgtatttcaatcaaagatacccgcggtattatcgatgcgattttggacggttcaatcgaaaaagcggaaatgggcgaattgccaatctttaatttagcgattcctaaagcattaccgggtgttgatcctgctattttggatccgcgcgatacttacgcagacaaagcgcaatggcaagttaaagcggaagatttggcaaaccgtttcgtgaaaaactttgtgaaatatacggcgaatccggaagcggctaaattagttggcgccggtccaaaagcataaaactgtaaaagcatagtatgtgcatgattcggtaaactaccgaataaaatctgaaaaataaaggctgagtatttccactcagccttttgttttggaaattagaagttgtttagaaagataaacggcgcggcttattcgtccgtattatttggggacataaaaaacccgttgataaacaacgggtttgtcatcttaacaggttattaggccgccggcacaaatcctactgcgtcataaaccgcc

SEQ ID NO: 3 - DNA, 1221 bp, Artificial sequence
  Synthesized mdh1
cggcaattaaatgcataaacgctaaacttgcgtgactacacattcttgagatgtggtcattgtaaacggcaattttgtggattaaggtcgcggcagcggagcaacatatcttagtttatcaatataataaggagtttaggatgaaagtcgcagtcctcggcgctgctggcggtattggccaggcgcttgcactactgttaaaaacccaactgccttcaggttcagaactctctctgtatgatatcgctccagtgactcccggtgtggctgtcgatctgagccatatccctactgctgtgaaaatcaaaggtttttctggtgaagatgcgactccggcgctggaaggcgcagatgtcgttcttatctctgcaggcgtagcgcgtaaaccgggtatggatcgttccgacctgtttaacgttaacgccggcatcgtgaaaaacctggtacagcaagttgcgaaaacctgcccgaaagcgtgcattggtattatcactaacccggttaacaccacagttgcaattgctgctgaagtgctgaaaaaagccggtgtttatgacaaaaacaaactgttcggcgttaccacgctggatatcattcgttccaacacctttgttgcggaactgaaaggcaaacagccaggcgaagttgaagtgccggttattggcggtcactctggtgttaccattctgccgctgctgtcacaggttcctggcgttagttttaccgagcaggaagtggctgatctgaccaaacgcatccagaacgcgggtactgaagtggttgaagcgaaggccggtggcgggtctgcaaccctgtctatgggccaggcagctgcacgttttggtctgtctctggttcgtgcactgcagggcgaacaaggcgttgtcgaatgtgcctacgttgaaggcgacggtcagtacgcccgtttcttctctcaaccgctgctgctgggtaaaaacggcgtggaagagcgtaaatctatcggtaccctgagcgcatttgaacagaacgcgctggaaggtatgctggatacgctgaagaaagatatcgccctgggcgaagagttcgttaataagtaattgattagcggataataaaaaaccggagcacagactccggttttttgttttgagcgcacgacttaattggttgccggatattcctgaatggtgacctgcagcgttaactgcttatcatcacgcatcactactacagggatca

SEQ ID NO: 4 - DNA, 1474 bp, Artificial sequence
  Synthesized mdh2
agatttatcgatatgagataaagattgctgcatgattctccttctgattctttttccctgtatatattttctccccttctgtataaatcgtacagtcagaagtagtccagaatatagtgctgcagactattacaaaagttcaatacaatatcataaaagttatagtaacatgcctcactcagttacaccatccatagaacaagattcgttaaaaattgccattttaggtgctgccggtggtatcgggcagtcgttatcgctgcttttgaaagctcagttgcaataccagttaaaggagagcaaccggagcgttacccacattcatctggctctttacgatgtcaaccaagaagccatcaacggtgttaccgccgacttgtctcatatagacacccccatttccgtgtcgagccactctcctgcaggtggcattgagaactgtttgcataacgcttctattgttgtcattcctgcaggtgttccaagaaaacctggcatgactcgtgatgacttatttaacgtgaatgctggtatcattagccagctcggtgattctattgcagaatgttgtgatctttccaaggtcttcgttcttgtcatttccaaccctgttaattctttagtcccagtgatggtttctaacattcttaagaaccatcctcagtctagaaattccggcattgaaagaaggatcatgggtgtcaccaagctcgacattgtcagagcgtccacttttctacgtgagataaacattgagtcagggctaactcctcgtgttaactccatgcctgacgtccctgtaattggcgggcattctggcgagactattattccgttgttttcacagtcaaacttcctatcgagattaaatgaggatcaattgaaatatttaatacatagagtccaatacggtggtgatgaagtggtcaaggccaagaacggtaaaggtagtgctaccttatcgatggcccatgccggttataagtgtgttgtccaatttgtttctttgttattgggtaacattgagcagatccatggaacctactatgtgccattaaaagatgcgaacaacttccccattgctcctggggcagatcaattattgcctctggtggacggtgcagactactttgccataccattaactattactacaaagggtgtttcctatgtggattatgacatcgttaataggatgaacgacatggaacgcaaccaaatgttgccaatttgcgtctcccagttaaagaaaaatatcgataagggcttggaattcgttgcatcgagatctgcatcatcttaatgagcatcggaccgaagcataagaatgcagcaaattgataatattcccgttaagccagtcgtaggaaaaggaaaagaatgagtagcaaatgtcacacaacaaacgcgcaccatccctacatacacaaacacacacacacacacacatatatatatattctaccccatataa

SEQ ID NO: 5 - DNA, 2141 bp, Artificial sequence
  Synthesized mqo
cgcgtgatgccgttgcccggacggcataatattacgacgctgacctggcggactgctgccgtcaggtcaatatgctaaaaatcccttcatttctaataaccctataattaatcaacgaaattataatgtttctaaaattagaatataatttataaacattatttaaatgttgttacttaagtgttaccgttgatgccgcgcaaatctcacctgcaataaagcgactaaaagtaaggcattaacaagatgaaaaaagtgactgccatgctcttctcgatggccgtggggcttaatgccgtttcgatggcggcaaaagcgaaagcgtccgaggagcaggaaactgatgtactgttgattggcggcggcattatgagcgccacgttggggacctatttacgcgagctggagcctgaatggtcgatgaccatggtggagcgcctggagggtgtcgcgcaggagagttcgaacggctggaataacgccggaaccgggcattctgcactgatggaactgaactacaccccgcaaaacgccgatggcagcatcagtattgaaaaagcagtcgccattaacgaagcatttcagatttcccgccagttctgggcgcaccaggttgagcgcggcgtgctgcgtactccgcgttcatttatcaataccgttccgcatatgagctttgtctggggcgaggataacgtcaatttcctgcgcgcccgttacgccgcgttgcaacaaagctcgctgtttcgcggtatgcgttactctgaagatcacgcgcagatcaaagagtgggcaccgttagtgatggaagggcgcgatccgcaacagaaagtggcagccacgcgtacggaaattggtaccgatgtgaactacggcgagatcacccgccagttaattgcttccttgcagaagaaatctaacttctcgctgcaactcagcagcgaagtccgcgccctaaagcgtaatgacgataacacctggaccgttaccgttgccgatctgaaaaatggcactgcacagaacattcgtgccaaatttgtctttatcggcgcgggcggtgcggcgctgaagctgttacaggaatcggggattccggaagcgaaagactacgccggtttcccggtgggcggacagttccttgtttcggaaaacccggacgtggttaatcaccatctggcgaaggtttacggtaaagcatccgttggcgcaccaccgatgtcggttccgcatatcgatacccgcgttctggacggtaaacgcgtagtgctgtttgggccatttgccaccttctcaaccaaattcctcaaaaacggttcattgtgggatctaatgagttccaccaccacctctaacgtgatgccgatgatgcacgtcgggctggataatttcgatctggtgaaatatctggtgagtcaggtgatgttgagtgaagaggatcgttttgaagcgttgaaagagtactatccgcaggcgaaaaaagaggactggcgtttgtggcaagcggggcagcgcgtgcagattatcaagcgtgatgccgagaaaggtggcgtactgcgtctgggtactgaagtcgtcagtgaccagcaaggaaccattgccgcgctcctgggggcatcgccaggggcgtcaaccgccgcgccgattatgttgaatctgctggaaaaagtatttggcgatcgtgtttccagcccgcaatggcaggctacgttgaaagcgatcgttccgtcttatggacgcaagctgaacggtgatgtagcggcaacagaacgcgagttgcagtacaccagcgaagtgctggggttgaactacgacaagccgcaggcagcagatagtacgccgaaaccgcagttgaaaccgcaacccgttcaaaaagaagtggcggatattgcgttgtaatgatacgccacatccggcatggtatgccggatgtggcgtatgctgataagacgcgccagcgtcgcatcaggcaaccggctcgggcgttagatgttggatgcggcgtaaccgccttatccaacctacggggtggagcacttcacagcggtgtaggcctgataagacgcgccagcgtcgcatcaggcaaccggcttgggcgttagatgtcggatgcggcgtaaccgccttatccgacctacggggtggag

SEQ ID NO: 6 - DNA, 1907 bp, Artificial sequence
  Synthesized fum1
cactgttgtagacgttaaacaagcatccaacaggaggccggaccgaacacacgggggagtgactctagctctttcttctgggatattcatatcgtttctccattacatacagtcatttttttgcatttttaaattcgtgacttttagtactgcagctgttttttttctcacgtcaagaaattccataaagtctaactattaaacggataagagatacaatcatgttgagatttaccaattgtagttgcaagactttcgtaaaatcgtcatataagcttaatataagaagaatgaactcctcgttcagaactgaaaccgatgcatttggtgagatacacgtgcctgctgataagtactggggtgcccaaactcaaagatcctttcaaaacttcaagattggtggcgctcgtgaaagaatgccattgcctttggtgcatgcatttggtgttttgaagaaatctgccgcgattgtgaatgagtctctggggggattggatcccaagatctccaaggctattcaacaggccgctgacgaagtggcttctggtaagctagacgaccactttccattggttgttttccaaacgggttccggtacccagtctaacatgaatgctaatgaagttatttccaatcgtgccattgagatcctaggaggcaagattggatctaaacaagtccatccaaacaatcattgcaaccaatctcaatcatccaatgatactttccccactgtcatgcatatcgctgccagtttgcaaattcaaaacgagttgatacctgagttgaccaatttaaagaacgcccttgaagccaaatccaaggaatttgaccatattgtgaagatcggtagaacacacttgcaagatgccacgcctttgacactaggtcaagagttttccggttacgtgcaacaagttgagaacggtatccaaagagtggcacattctttgaaaacattgagtttcctggcacaaggtggtactgccgttggtacagggttgaacaccaagcccggattcgatgtcaagatagccgagcaaatttccaaagaaactggactaaagtttcaaaccgcccctaataagttcgaggctttggctgctcacgacgccattgtcgaatgtagcggtgctctaaacacccttgcttgttctcttttcaaaatagcgcaagatataagatacttagggtccgggccacgttgcggctaccatgaactaatgctgccagagaatgaaccaggttcctcaatcatgcctggtaaagttaacccaacccaaaacgaggcattgactcaagtgtgtgtgcaagtcatgggtaacaatgcagctatcacgtttgccggctctcaaggtcaattcgagttaaatgtcttcaagccagtcatgatcgccaatctattgaattcgatcaggttaattactgatgccgcatattcatttagagtgcactgtgttgaaggtatcaaagccaatgagcctcgtattcatgagttgttgactaaatctttaatgttagtcaccgctttgaaccctaagatcggttatgatgccgcttccaaggtcgccaagaatgctcataagaagggcattaccttgaaggaaagtgcattggaattgggtgtattgactgaaaaggaatttgatgaatgggttgttcctgaacacatgctaggtcctaaataaacgagctaaatacctaataatatacaagttttttatgtcttattatatgaaggaaaataaggaagtggagagaatttgtgattcagcaatggtccctccgctaaggtcccgcctctggccagttatgtcagaagaggagcataggcatacattccatttttgatggcttaatggggcccataaatttacatcactacattattattcctatacaagtta

SEQ ID NO: 7 - DNA, 2141 bp, Artificial sequence
  Synthesized fumA
tttgctgccccaggcatagttttgcactgagttaatgagtttttgcatgatcaatccctgttttaatgtggaaattaatcccactattaaagcaagaatcctacgggaagtaacctggagccgcaaaaagtcgtactagtctcagtttttgttaaaaaagtgtgtaggatattgttactcgcttttaacagggcaacggaacacccgcccagagcataaccaaaccaggcagtaagtgagagaacaatgtcaaacaaaccctttcattatcaggctccttttccactcaaaaaagatgatactgagtattacctgctaaccagcgaacacgttagcgtatctgaatttgaagggcaggagattttgaaagtcgcacccgaagcgttaactctgttggcgcgccaggcgtttcatgatgcgtcgttcatgctgcgtccggcgcaccaacaacaggtggccgacattctgcgtgacccggaggccagcgaaaatgataaatatgtggcgctgcaattcctgcgtaactccgacatcgcggcgaaaggcgttctgccaacctgtcaggataccggcaccgcgattattgttggtaaaaaagggcagcgtgtatggaccggtggtggtgatgaagcggcgctggcgcgcggtgtctataacacttatatcgaagataatctgcgctactcgcaaaacgcgccgctggatatgtataaagaagtgaataccggcaccaatctgccagcgcagatcgatctttatgccgttgatggcgacgaatacaaattcctctgtatcgccaaaggtggtggttcggcaaacaagacgtatctctatcaggaaaccaaagcgttactgacgccggggaaactgaaaaattacctggttgagaagatgcgcacgctgggtacggcggcctgtcctccgtatcatattgcgttcgttattggtggaacttctgcagaaacgaaccttaaaacggtgaaactggcttccgcgaaatactatgatgaactgccaacggaagggaatgagcacggtcaggcgttccgcgatgtggaactggaaaaagaattgctgatcgaagcgcaaaatcttggtctgggtgcgcagtttggtggtaaatacttcgctcacgacatccgcgtgattcgcctgccacgtcacggcgcatcctgcccggtcggtatgggcgtctcctgctctgctgaccgtaatatcaaagcgaagatcaaccgtcaggggatctggatcgaaaaactggaacataatccaggcaaatatatcccggaagagctgcgcaaagcgggagaaggcgaagcggtgcgcgttgaccttaaccgtccgatgaaagagatcctcgcacagttgtcgcagtatcccgtttctacacgcttatcgcttaacggcacgattatcgtcggtcgtgatattgctcacgccaaactgaaagagcggatggataacggtgaagggctgccgcagtacatcaaagatcatccgatttactacgcgggtccggccaaaacgccggaaggttatgcctccggttctcttggcccaacgaccgccggacggatggattcttatgtcgatcaactgcaagcgcagggcggaagtatgatcatgctggcgaaaggcaaccgcagccagcaggtgacggatgcctgtaaaaaacacggcggcttctaccttggcagtatcggtggtccggccgctgtattggcgcagggaagtattaagagcctggaatgtgttgaatatccggaactgggaatggaagccatctggaaaattgaagtggaagatttcccggcgtttatccttgtggatgataaaggaaatgacttcttccagcagatacaactcacacaatgcacccgctgtgtgaaataaacagagccgcccttcggggcggtttttttacatggcacgaaagaccaaacatttgttatcaaatggtaaataataagtgagctaaaagttgcttaacgaaagcaaaacagaaagaaaaaattaatcaggtgaggagcaggtcatgaatacagtacgcagcgaaaaagattcgatgggggcgattgatgtcccggcagataagctgtggggcgcacaaactcaacgctcgctggagcatttccgcattt

SEQ ID NO: 8 - DNA, 2141 bp, Artificial sequence
  Synthesized fumB
cctgccgacttatccgagcgatctggcagcgattcagtttgaccgttccggcaccacccacatcggtcgcttcgtcatcaaccacagctttattctgccggggttgattggtgtgagcgtatcgtgcgtcttcggctggatcttcgccgcgatgtacgggttcttataaatgcactttgcgtgccgcccgtgactacgcggcacgccattttcgaataacaaatacagagttacaggctggaagctatgtcaaacaaaccctttatctaccaggcacctttcccgatggggaaagacaataccgaatactatctactcacttccgattacgttagcgttgccgacttcgacggcgaaaccatcctgaaagtggaaccagaagccctgaccctgctggcgcagcaagcctttcacgacgcttcttttatgctccgcccggcacaccagaaacaggttgcggctattcttcacgatccagaagccagcgaaaacgacaagtacgtggcgctgcaattcttaagaaactccgaaatcgccgccaaaggcgtgctgccgacctgccaggataccggcaccgcgatcatcgtcggtaaaaaaggccagcgcgtgtggaccggcggcggtgatgaagaaacgctgtcgaaaggcgtctataacacctatatcgaagataacctgcgctattcacagaatgcggcgctggacatgtacaaagaggtcaacaccggcactaacctgcctgcgcaaatcgacctgtacgcggtagatggcgatgagtacaaattcctttgcgttgcgaaaggcggcggctctgccaacaaaacgtatctctaccaggaaaccaaagccctgctgactcccggcaaactgaaaaacttcctcgtcgagaaaatgcgtaccctcggtactgcagcctgcccgccgtaccatatcgcgtttgtgattggcggtacgtctgcggaaaccaacctgaaaaccgtcaagttagcaagcgctcactattacgatgaactgccgacggaagggaacgaacatggtcaggcgttccgcgatgtccagctggaacaggaactgctggaagaggcccagaaactcggtcttggcgcgcagtttggcggtaaatacttcgcgcacgacattcgcgttatccgtctgccacgtcacggcgcatcctgcccggtcggcatgggcgtctcctgctccgctgaccgtaacattaaagcgaaaatcaaccgcgaaggtatctggatcgaaaaactggaacacaacccaggccagtacattccacaagaactgcgccaggccggtgaaggcgaagcggtgaaagttgaccttaaccgcccgatgaaagagatcctcgcccagctttcgcaatacccggtatccactcgtttgtcgctcaccggcaccattatcgtgggccgagatattgcacacgccaagctgaaagagctgattgacgccggtaaagaacttccgcagtacatcaaagatcacccgatctactacgcgggtccggcgaaaacccctgccggttatccatcaggttcacttggcccaaccaccgcaggccgtatggactcctacgtggatctgctgcaatcccacggcggcagcatgatcatgctggcgaaaggtaaccgcagtcagcaggttaccgacgcgtgtcataaacacggcggcttctacctcggtagcatcggcggtccggcggcggtactggcgcagcagagcatcaagcatctggagtgcgtcgcttatccggagctgggtatggaagctatctggaaaatcgaagtagaagatttcccggcgtttatcctggtcgatgacaaaggtaacgacttcttccagcaaatcgtcaacaaacagtgcgcgaactgcactaagtaacctcttcggcccagcgcctggcagcatgctgccaggtgatccccctggccacctcttttgcgattgtaatttcacgcttgctggtgaatagtcagtattttccctgatttgcgaactcaccatgacccgcacactcaagccgttaattcttaacaccagcgcactgacgctaacgttaatcctgatttataccggcatttcggcccatgacaaactcacctggctgatggaagtgacaccggtgatta

SEQ ID NO: 9 - DNA, 1824 bp, Artificial sequence
  Synthesized fumC
ggatgataaaggaaatgacttcttccagcagatacaactcacacaatgcacccgctgtgtgaaataaacagagccgcccttcggggcggtttttttacatggcacgaaagaccaaacatttgttatcaaatggtaaataataagtgagctaaaagttgcttaacgaaagcaaaacagaaagaaaaaattaatcaggtgaggagcaggtcatgaatacagtacgcagcgaaaaagattcgatgggggcgattgatgtcccggcagataagctgtggggcgcacaaactcaacgctcgctggagcatttccgcatttcgacggagaaaatgcccacctcactgattcatgcgctggcgctaaccaagcgtgcagcggcaaaagttaatgaagatttaggcttgttgtctgaagagaaagcgagcgccattcgtcaggcggcggatgaagtactggcaggacagcatgacgacgaattcccgctggctatctggcagaccggctccggcacgcaaagtaacatgaacatgaacgaagtgctggctaaccgggccagtgaattactcggcggtgtgcgcgggatggaacgtaaagttcaccctaacgacgacgtgaacaaaagccaaagttccaacgatgtctttccgacggcgatgcacgttgcggcgctgctggcgctgcgcaagcaactcattcctcagcttaaaaccctgacacagacactgaatgagaaatcccgtgcttttgccgatatcgtcaaaattggtcgtactcacttgcaggatgccacgccgttaacgctggggcaggagatttccggctgggtagcgatgctcgagcataatctcaaacatatcgaatacagcctgcctcacgtagcggaactggctcttggcggtacagcggtgggtactggactaaatacccatccggagtatgcgcgtcgcgtagcagatgaactggcagtcattacctgtgcaccgtttgttaccgcgccgaacaaatttgaagcgctggcgacctgtgatgccctggttcaggcgcacggcgcgttgaaagggttggctgcgtcactgatgaaaatcgccaatgatgtccgctggctggcctctggcccgcgctgcggaattggtgaaatctcaatcccggaaaatgagccgggcagctcaatcatgccggggaaagtgaacccaacacagtgtgaggcattaaccatgctctgctgtcaggtgatggggaacgacgtggcgatcaacatggggggcgcttccggtaactttgaactgaacgtcttccgtccaatggtgatccacaatttcctgcaatcggtgcgcttgctggcagatggcatggaaagttttaacaaacactgcgcagtgggtattgaaccgaatcgtgagcgaatcaatcaattactcaatgaatcgctgatgctggtgactgcgcttaacacccacattggttatgacaaagccgccgagatcgccaaaaaagcgcataaagaagggctgaccttaaaagctgcggcccttgcgctggggtatcttagcgaagccgagtttgacagctgggtacggccagaacagatggtcggcagtatgaaagccgggcgttaatctgcaacatacaggtgcagccgtggaatgatcaaacgaagcggctgcgcctgaggtttataacggtgctgcacattgtcggcatcgtaatttaacaactcaccaacgtccggcacgcccgcgccattatcccgattaatcagtgcaatcagtggtgtagggcaggcgtgttggacttgtttttgatctcctttgtaccagacgcgggcaa

SEQ ID NO: 10 - DNA, 2351 bp, Artificial sequence
  Synthesized frdA
gagacaaattttacgcaggaatcaaacagcggtgggcagtgactaaaaaaagcacgatctgatggtttagtaattaaattaatcatcttcagtgataatttagccctcttgcgcactaaaaaaatcgatctcgtcaaatttcagacttatccatcagactatactgttgtacctataaaggagcagtggaatagcgttcgcagaccgtaactttcaggtacttaccctgaagtacgtggctgtgggataaaaacaatctggaggaatgtcgtgcaaacctttcaagccgatcttgccattgtaggcgccggtggcgcgggattacgtgctgcaattgctgccgcgcaggcaaatccgaatgcaaaaatcgcactaatctcaaaagtatacccgatgcgtagccataccgttgctgcagaagggggctccgccgctgtcgcgcaggatcatgacagcttcgaatatcactttcacgatacagtagcgggtggcgactggttgtgtgagcaggatgtcgtggattatttcgtccaccactgcccaaccgaaatgacccaactggaactgtggggatgcccatggagccgtcgcccggatggtagcgtcaacgtacgtcgcttcggcggcatgaaaatcgagcgcacctggttcgccgccgataagaccggcttccatatgctgcacacgctgttccagacctctctgcaattcccgcagatccagcgttttgacgaacatttcgtgctggatattctggttgatgatggtcatgttcgcggcctggtagcaatgaacatgatggaaggcacgctggtgcagatccgtgctaacgcggtcgttatggctactggcggtgcgggtcgcgtttatcgttacaacaccaacggcggcatcgttaccggtgacggtatgggtatggcgctaagccacggcgttccgctgcgtgacatggaattcgttcagtatcacccaaccggtctgccaggttccggtatcctgatgaccgaaggttgccgcggtgaaggcggtattctggtcaacaaaaatggctaccgttatctgcaagattacggcatgggcccggaaactccgctgggcgagccgaaaaacaaatatatggaactgggtccacgcgacaaagtctctcaggccttctggcacgaatggcgtaaaggcaacaccatctccacgccgcgtggcgatgtggtttatctcgacttgcgtcacctcggcgagaaaaaactgcatgaacgtctgccgttcatctgcgaactggcgaaagcgtacgttggcgtcgatccggttaaagaaccgattccggtacgtccgaccgcacactacaccatgggcggtatcgaaaccgatcagaactgtgaaacccgcattaaaggtctgttcgccgtgggtgaatgttcctctgttggtctgcacggtgcaaaccgtctgggttctaactccctggcggaactggtggtcttcggccgtctggccggtgaacaagcgacagagcgtgcagcaactgccggtaatggcaacgaagcggcaattgaagcgcaggcagctggcgttgaacaacgtctgaaagatctggttaaccaggatggcggcgaaaactgggcgaagatccgcgacgaaatgggcctggctatggaagaaggctgcggtatctaccgtacgccggaactgatgcagaaaaccatcgacaagctggcagagctgcaggaacgcttcaagcgcgtgcgcatcaccgacacttccagcgtgttcaacaccgacctgctctacaccattgaactgggccacggtctgaacgttgctgaatgtatggcgcactccgcaatggcacgtaaagagtcccgcggcgcgcaccagcgtctggacgaaggttgcaccgagcgtgacgacgtcaacttcctcaaacacaccctcgccttccgcgatgctgatggcacgactcgcctggagtacagcgacgtgaagattactacgctgccgccagctaaacgcgtttacggtggcgaagcggatgcagccgataaggcggaagcagccaataagaaggagaaggcgaatggctgagatgaaaaacctgaaaattgaggtggtgcgctataacccggaagtcgataccgcaccgcatagcgcattctatgaagtgccttatgacgcaactacctcattactggatgcgctgggctacatcaaagacaacctggcaccggacctgagctaccgctggtcctgccgtatggcgatttgtggttcctgcggcatgatggttaacaacgtgccaaaactggcatgtaaaaccttcctgcgtgattacaccgacggtatgaaggttgaagcgt

SEQ ID NO: 11 - DNA, 955 bp, Artificial sequence
  Synthesized frdB
gagtacagcgacgtgaagattactacgctgccgccagctaaacgcgtttacggtggcgaagcggatgcagccgataaggcggaagcagccaataagaaggagaaggcgaatggctgagatgaaaaacctgaaaattgaggtggtgcgctataacccggaagtcgataccgcaccgcatagcgcattctatgaagtgccttatgacgcaactacctcattactggatgcgctgggctacatcaaagacaacctggcaccggacctgagctaccgctggtcctgccgtatggcgatttgtggttcctgcggcatgatggttaacaacgtgccaaaactggcatgtaaaaccttcctgcgtgattacaccgacggtatgaaggttgaagcgttagctaacttcccgattgaacgcgatctggtggtcgatatgacccacttcatcgaaagtctggaagcgatcaaaccgtacatcatcggcaactcccgcaccgcggatcagggtactaacatccagaccccggcgcagatggcgaagtatcaccagttctccggttgcatcaactgtggtttgtgctacgccgcgtgcccgcagtttggcctgaacccagagttcatcggtccggctgccattacgctggcgcatcgttataacgaagatagccgcgaccacggtaagaaggagcgtatggcgcagttgaacagccagaacggcgtatggagctgtactttcgtgggctactgctccgaagtctgcccgaaacacgtcgatccggctgcggccattcagcagggcaaagtagaaagttcgaaagactttcttatcgcgaccctgaaaccacgctaaggagtgcaacatgacgactaaacgtaaaccgtatgtacggccaatgacgtccacctggtggaaaaaattgccgttttatcgcttttacatgctgcgcgaaggcacggcggt

SEQ ID NO: 12 - DNA, 514 bp, Artificial sequence
  Synthesized frdC
gtagaaagttcgaaagactttcttatcgcgaccctgaaaccacgctaaggagtgcaacatgacgactaaacgtaaaccgtatgtacggccaatgacgtccacctggtggaaaaaattgccgttttatcgcttttacatgctgcgcgaaggcacggcggttccggctgtgtggttcagcattgaactgattttcgggctgtttgccctgaaaaatggcccggaagcctgggcgggattcgtcgactttttacaaaacccggttatcgtgatcattaacctgatcactctggcggcagctctgctgcacaccaaaacctggtttgaactggcaccgaaagcggccaatatcattgtaaaagacgaaaaaatgggaccagagccaattatcaaaagtctctgggcggtaactgtggttgccaccatcgtaatcctgtttgttgccctgtactggtaaggagcctgagatgattaatccaaatccaaagcgttctgacgaaccggtattctggggcct

SEQ ID NO: 13 - DNA, 468 bp, Artificial sequence
  Synthesized frdD
ggttgccaccatcgtaatcctgtttgttgccctgtactggtaaggagcctgagatgattaatccaaatccaaagcgttctgacgaaccggtattctggggcctcttcggggccggtggtatgtggagcgccatcattgcgccggtgatgatcctgctggtgggtattctgctgccactggggttgtttccgggtgatgcgctgagctacgagcgcgttctggcgttcgcgcagagcttcattggtcgcgtattcctgttcctgatgatcgttctgccgctgtggtgtggtttacaccgtatgcaccacgcgatgcacgatctgaaaatccacgtacctgcgggcaaatgggttttctacggtctggctgctatcctgacagttgtcacgctgattggtgtcgttacaatctaacgcatcgccaatgtaaatccggcccgcctatggcgggccgttttgtatggaaacc

SEQ ID NO: 14 - DNA, 2297 bp, Artificial sequence
  Synthesized sdhA
ctctacatcatttatatggtcggttttttcgctaccagtggcgagctgacatatgaagtctggatcggtttcttcgcctctgcgttcaccaaagtgttcaccctgctggcgctgttttctatcttgatccatgcctggatcggcatgtggcaggtgttgaccgactacgttaaaccgctggctttgcgcctgatgctgcaactggtgattgtcgttgcactggtggtttacgtgatttatggattcgttgtggtgtggggtgtgtgatgaaattgccagtcagagaatttgatgcagttgtgattggtgccggtggcgcaggtatgcgcgcggcgctgcaaatttcccagagcggccagacctgtgcgctgctctctaaagtcttcccgacccgttcccataccgtttctgcgcaaggcggcattaccgttgcgctgggtaatacccatgaagataactgggaatggcatatgtacgacaccgtgaaagggtcggactatatcggtgaccaggacgcgattgaatatatgtgtaaaaccgggccggaagcgattctggaactcgaacacatgggcctgccgttctcgcgtctcgatgatggtcgtatctatcaacgtccgtttggcggtcagtcgaaaaacttcggcggcgagcaggcggcacgcactgcggcagcagctgaccgtaccggtcacgcactgttgcacacgctttatcagcagaacctgaaaaaccacaccaccattttctccgagtggtatgcgctggatctggtgaaaaaccaggatggcgcggtggtgggttgtaccgcactgtgcatcgaaaccggtgaagtggtttatttcaaagcccgcgctaccgtgctggcgactggcggagcagggcgtatttatcagtccaccaccaacgcccacattaacaccggcgacggtgtcggcatggctatccgtgccggcgtaccggtgcaggatatggaaatgtggcagttccacccgaccggcattgccggtgcgggcgtactggtcaccgaaggttgccgtggtgaaggcggttatctgctgaacaaacatggcgaacgttttatggagcgttatgcgccgaacgccaaagacctggcgggccgtgacgtggttgcgcgttccatcatgatcgaaatccgtgaaggtcgcggctgtgatggtccgtgggggccacacgcgaaactgaaactcgatcacctgggtaaagaagttctcgaatcccgtctgccgggtatcctggagctttcccgtaccttcgctcacgtcgatccggtgaaagagccgattccggttatcccaacctgtcactacatgatgggcggtattccgaccaaagttaccggtcaggcactgactgtgaatgagaaaggcgaagatgtggttgttccgggactgtttgccgttggtgaaatcgcttgtgtatcggtacacggcgctaaccgtctgggcggcaactcgctgctggacctggtggtctttggtcgcgcggcaggtctgcatctgcaagagtctatcgccgagcagggcgcactgcgcgatgccagcgagtctgatgttgaagcgtctctggatcgcctgaaccgctggaacaataatcgtaacggtgaagatccggtggcgatccgtaaagcgctgcaagaatgtatgcagcataacttctcggtcttccgtgaaggtgatgcgatggcgaaagggcttgagcagttgaaagtgatccgcgagcgtctgaaaaatgcccgtctggatgacacttccagcgagttcaacacccagcgcgttgagtgcctggaactggataacctgatggaaacggcgtatgcaacggctgtttctgccaacttccgtaccgaaagccgtggcgcgcatagccgcttcgacttcccggatcgtgatgatgaaaactggctgtgccactccctgtatctgccagagtcggaatccatgacgcgccgaagcgtcaacatggaaccgaaactgcgcccggcattcccgccgaagattcgtacttactaatgcggagacaggaaaatgagactcgagttttcaatttatcgctataacccggatgttgatgatgctccgcgtatgcaggattacaccctggaagcggatgaaggtcgcgacatgatgctgctggatgcgcttatccagctaaaagagaaagatcccagcctgtcgttccgccgctcctgccgtgaaggtgtgtgcggttccgacggtctgaacatgaacggcaagaatggtctggcctgtattaccccgatttcggcactcaac

SEQ ID NO: 15 - DNA, 931 bp, Artificial sequence
  Synthesized sdhB
tatctgccagagtcggaatccatgacgcgccgaagcgtcaacatggaaccgaaactgcgcccggcattcccgccgaagattcgtacttactaatgcggagacaggaaaatgagactcgagttttcaatttatcgctataacccggatgttgatgatgctccgcgtatgcaggattacaccctggaagcggatgaaggtcgcgacatgatgctgctggatgcgcttatccagctaaaagagaaagatcccagcctgtcgttccgccgctcctgccgtgaaggtgtgtgcggttccgacggtctgaacatgaacggcaagaatggtctggcctgtattaccccgatttcggcactcaaccagccgggcaagaagattgtgattcgcccgctgccaggtttaccggtgatccgcgatttggtggtagacatgggacaattctatgcgcaatatgagaaaattaagccttacctgttgaataatggacaaaatccgccagctcgcgagcatttacagatgccagagcagcgcgaaaaactcgacgggctgtatgaatgtattctctgcgcatgttgttcaacctcttgtccgtctttctggtggaatcccgataagtttatcggcccggcaggcttgttagcggcatatcgtttcctgattgatagccgtgataccgagactgacagccgcctcgacggtttgagtgatgcattcagcgtattccgctgtcacagcatcatgaactgcgtcagtgtatgtccgaaggggctgaacccgacgcgcgccatcggccatatcaagtcgatgttgttgcaacgtaatgcgtaaaccgtaggcctgataagacgcgcaagcgtcgcatcaggcaaccagtgccggatgcggcgtgaacgccttatccggcctacaagtcattacccgtaggcctgataag

SEQ ID NO: 16 - DNA, 506 bp, Artificial sequence
  Synthesized sdhC
gttaacctctgtgcccgtagtccccagggaataataagaacagcatgtgggcgttattcatgataagaaatgtgaaaaaacaaagacctgttaatctggacctacagaccatccggttccccatcacggcgatagcgtccattctccatcgcgtttccggtgtgatcacctttgttgcagtgggcatcctgctgtggcttctgggtaccagcctctcttcccctgaaggtttcgagcaagcttccgcgattatgggcagcttcttcgtcaaatttatcatgtggggcatccttaccgctctggcgtatcacgtcgtcgtaggtattcgccacatgatgatggattttggctatctggaagaaacattcgaagcgggtaaacgctccgccaaaatctcctttgttattactgtcgtgctttcacttctcgcaggagtcctcgtatggtaagcaacgcctccgcattaggacgcaatggcgtacatgatttcatcctcgttcgcgcta

SEQ ID NO: 17 - DNA, 452 bp, Artificial sequence
  Synthesized sdhD
aaaatctcctttgttattactgtcgtgctttcacttctcgcaggagtcctcgtatggtaagcaacgcctccgcattaggacgcaatggcgtacatgatttcatcctcgttcgcgctaccgctatcgtcctgacgctctacatcatttatatggtcggttttttcgctaccagtggcgagctgacatatgaagtctggatcggtttcttcgcctctgcgttcaccaaagtgttcaccctgctggcgctgttttctatcttgatccatgcctggatcggcatgtggcaggtgttgaccgactacgttaaaccgctggctttgcgcctgatgctgcaactggtgattgtcgttgcactggtggtttacgtgatttatggattcgttgtggtgtggggtgtgtgatgaaattgccagtcagagaatttgatgcagttgtgattggtgccggtggcg

SEQ ID NO: 18 - DNA, 2967 bp, Artificial sequence
  Synthesized pflB
aagcgttcattatggtgctgccggtcgcgatgtttgttgccagcggttttgagcacagtatcgcaaacatgtttatgatcccgatgggtattgtaatccgcgacttcgcatccccggaattttggaccgcagtcggttctgcaccggaaaatttttctcacctgaccgtgatgaatttcatcactgataacctgattccggttacgatcggcaacattatcggtggtggtttgttggttgggttgacatactgggtcatttacctgcgtgaaaacgaccaccattaatggttgtcgaagtacgcagtaaataaaaaatccacttaagaaggtaggtgttacatgtccgagcttaatgaaaagttagccacagcctgggaaggttttaccaaaggtgactggcagaatgaagtaaacgtccgtgacttcattcagaaaaactacactccgtacgagggtgacgagtccttcctggctggcgctactgaagcgaccaccaccctgtgggacaaagtaatggaaggcgttaaactggaaaaccgcactcacgcgccagttgactttgacaccgctgttgcttccaccatcacctctcacgacgctggctacatcaacaagcagcttgagaaaatcgttggtctgcagactgaagctccgctgaaacgtgctcttatcccgttcggtggtatcaaaatgatcgaaggttcctgcaaagcgtacaaccgcgaactggatccgatgatcaaaaaaatcttcactgaataccgtaaaactcacaaccagggcgtgttcgacgtttacactccggacatcctgcgttgccgtaaatctggtgttctgaccggtctgccagatgcatatggccgtggccgtatcatcggtgactaccgtcgcgttgcgctgtacggtatcgactacctgatgaaagacaaactggcacagttcacttctctgcaggctgatctggaaaacggcgtaaacctggaacagactatccgtctgcgcgaagaaatcgctgaacagcaccgcgctctgggtcagatgaaagaaatggctgcgaaatacggctacgacatctctggtccggctaccaacgctcaggaagctatccagtggacttacttcggctacctggctgctgttaagtctcagaacggtgctgcaatgtccttcggtcgtacctccaccttcctggatgtgtacatcgaacgtgacctgaaagctggcaagatcaccgaacaagaagcgcaggaaatggttgaccacctggtcatgaaactgcgtatggttcgcttcctgcgtactccggaatacgatgaactgttctctggcgacccgatctgggcaaccgaatctatcggtggtatgggcctcgacggtcgtaccctggttaccaaaaacagcttccgtttcctgaacaccctgtacaccatgggtccgtctccggaaccgaacatgaccattctgtggtctgaaaaactgccgctgaacttcaagaaattcgccgctaaagtgtccatcgacacctcttctctgcagtatgagaacgatgacctgatgcgtccggacttcaacaacgatgactacgctattgcttgctgcgtaagcccgatgatcgttggtaaacaaatgcagttcttcggtgcgcgtgcaaacctggcgaaaaccatgctgtacgcaatcaacggcggcgttgacgaaaaactgaaaatgcaggttggtccgaagtctgaaccgatcaaaggcgatgtcctgaactatgatgaagtgatggagcgcatggatcacttcatggactggctggctaaacagtacatcactgcactgaacatcatccactacatgcacgacaagtacagctacgaagcctctctgatggcgctgcacgaccgtgacgttatccgcaccatggcgtgtggtatcgctggtctgtccgttgctgctgactccctgtctgcaatcaaatatgcgaaagttaaaccgattcgtgacgaagacggtctggctatcgacttcgaaatcgaaggcgaatacccgcagtttggtaacaatgatccgcgtgtagatgacctggctgttgacctggtagaacgtttcatgaagaaaattcagaaactgcacacctaccgtgacgctatcccgactcagtctgttctgaccatcacttctaacgttgtgtatggtaagaaaacgggtaacaccccagacggtcgtcgtgctggcgcgccgttcggaccgggtgctaacccgatgcacggtcgtgaccagaaaggtgcagtagcctctctgacttccgttgctaaactgccgtttgcttacgctaaagatggtatctcctacaccttctctatcgttccgaacgcactgggtaaagacgacgaagttcgtaagaccaacctggctggtctgatggatggttacttccaccacgaagcatccatcgaaggtggtcagcacctgaacgttaacgtgatgaaccgtgaaatgctgctcgacgcgatggaaaacccggaaaaatatccgcagctgaccatccgtgtatctggctacgcagtacgtttcaactcgctgactaaagaacagcagcaggacgttattactcgtaccttcactcaatctatgtaattagatttgactgaaatcgtacagtaaaaagcgtacaataaaggctccacgaaagtggggccttttttagcgcgagagccttttttgtcagctatctatactttaaggtgactgccaaaacagactcgacgtagccttcgagctgcgcaccaacacggcctcagatgggccacatctggagaaacaccgcaatgtcagttattggtcgcattcactcctttgaatcctgtggaaccgtagacggcccaggtattcgctttatcacctttttccagggctgcctgatgcgctgcctgtattgtcataaccgcgacacctgggacacgcatggcggtaaagaagt

SEQ ID NO: 19 - DNA, 2792 bp, Artificial sequence
  Synthesized fdhF
gccagtgcttgccgaagagatgtcattactgatgctggacagccgccgggtgatccaaagcattcagttgatgaaatcgctgggcggcgggtatcaggcaggtcccgtcgtcgagaaaaaataaaatgtctgccgcgtgatggctgtcacgcggtatttcgtttcgtcacgtcaaaactgacgacagcctgtttttcgtcagagttttgaataaatagtgcccgtaatatcagggaatgaccccacataaaatgtggcataaaagatgcatactgtagtcgagagcgcgtatgcgtgatttgattaactggagcgagaccgatgaaaaaagtcgtcacggtttgcccctattgcgcatcaggttgcaaaatcaacctggtcgtcgataacggcaaaatcgtccgggcggaggcagcgcaggggaaaaccaaccagggtaccctgtgtctgaagggttattatggctgggacttcattaacgatacccagatcctgaccccgcgcctgaaaacccccatgatccgtcgccagcgtggcggcaaactcgaacctgtttcctgggatgaggcactgaattacgttgccgagcgcctgagcgccatcaaagagaagtacggtccggatgccatccagacgaccggctcctcgcgtggtacgggtaacgaaaccaactatgtaatgcaaaaatttgcgcgcgccgttattggtaccaataacgttgactgctgcgctcgtgtctgacacggcccatcggttgcaggtctgcaccaatcggtcggtaatggcgcaatgagcaatgctattaacgaaattgataataccgatttagtgttcgttttcgggtacaacccggcggattcccacccaatcgtggcgaatcacgtaattaacgctaaacgtaacggggcgaaaattatcgtctgcgatccgcgcaaaattgaaaccgcgcgcattgctgacatgcacattgcactgaaaaacggctcgaacatcgcgctgttgaatgcgatgggccatgtcattattgaagaaaatctgtacgacaaagcgttcgtcgcttcacgtacagaaggctttgaagagtatcgtaaaatcgttgaaggctacacgccggagtcggttgaagatatcaccggcgtcagcgccagtgagattcgtcaggcggcacggatgtatgcccaggcgaaaagcgccgccatcctgtggggcatgggtgtaacccagttctaccagggcgtggaaaccgtgcgttctctgaccagcctcgcgatgctgaccggtaacctcggtaagccgcatgcgggtgttaacccggttcgtggtcagaacaacgttcagggtgcctgcgatatgggcgcgctgccggatacgtatccgggataccagtacgtgaaagatccggctaaccgcgagaaattcgccaaagcctggggcgtggaaagcctgccagcgcataccggctatcgcatcagcgagctgccgcaccgcgcagcgcatggcgaagtgcgtgccgcgtacattatgggcgaagatccgctacaaactgacgcggagctgtcggcagtacgtaaagcctttgaagatctggaactggttatcgttcaggacatctttatgaccaaaaccgcgtcggcggcggatgttattttaccgtcaacgtcgtggggcgagcatgaaggcgtgtttactgcggctgaccgtggcttccagcgtttcttcaaggcggttgaaccgaaatgggatctgaaaacggactggcaaatcatcagtgaaatcgccacccgtatgggttatccgatgcactacaacaacacccaggagatctgggatgagttgcgtcatctgtgcccggatttctacggtgcgacttacgagaaaatgggcgaactgggcttcattcagtggccttgccgcgatacttcagatgccgatcaggggacttcttatctgtttaaagagaagtttgataccccgaacggtctggcgcagttcttcacctgcgactgggtagcgccaatcgacaaactcaccgacgagtacccgatggtactgtcaacggtgcgtgaagttggtcactactcttgccgttcgatgaccggtaactgtgcggcactggcggcgctggctgatgaacctggctacgcacaaatcaataccgaagacgccaaacgtctgggtattgaagatgaggcattggtttgggtgcactcgcgtaaaggcaaaattatcacccgtgcgcaggtcagcgatcgtccgaacaaaggggcgatttacatgacctaccagtggtggattggtgcctgtaacgagctggttaccgaaaacttaagcccgattacgaaaacgccggagtacaaatactgcgccgttcgcgtcgagccgatcgccgatcagcgcgccgccgagcagtacgtgattgacgagtacaacaagttgaaaactcgcctgcgcgaagcggcactggcgtaataccgtcctttctacagcctcctttcggaggctgtttttttatccattcgaactctttatactggttactccctacccaatcgtattatcaaaatgaaaaaaattatcgcattgatgttgtttttgacattctttgcccacgccaacgactccgagcctggcagccagtatttaaaggcagcagaggccggggaccgacgcgcacaatattttcttgccgacagctggtttagctccggcgatttgagcaaagccgaatattgggcacagaaagccgccgacagcggtgatgctgatgcctgcgcgctgctggcgcagatcaa

SEQ ID NO: 20 - DNA, 1200 bp, Artificial sequence
  Synthesized hycD
acctgcgtaaccggctgaacccggtgcagtgggtgccgggctggcagagtgaatgcctggcgggctcggtgcgccgtctggcctttattgaactggcgattctgctggtgattgtggtttcccggggagcataaaaacaatgactgtgattcttggtttgattcaggcgctggtgctgtttgccattgcgccgctgatatccggcattacccgcgtagcccgcgcccggatgcataaccgtcaggggccgggggtgcttcaggagtaccgggatctgttcaagctgctgggccgccagagcgtggccccggcggcctctggctgggtcttccgcctgatgccgtttgtgatggtgggcgttatgctaaccatcgccagcgccctgccgctgattactgtcgccgctccggtggcggcactgggtgaccttatcaccctgatttacctgtttgccattgcccgcttcttcttcgccattgccggtctggatacgggcagcccgtttaccggtatcggcgccagccgtgaagcgatgctcggggtactggtggagcctatcctgctgctgggcctgtgggtggccgcgcaggtcgcgggctctacccatatcagcaatatcaccctgaccctgtaccactggccggcctcccagacgctgaccctgctgctggccatggcggcctgcgccttcgccacctttattgagatgggcaagctgcccttcgacctggcggaagcagagcaggagctgcaggaagggccgctgactgaatacagcggatctggttttgccatcctcaaatggggtatcagcctcaagcagctggtggtgttgcagatgtttgtcggggtgttcctgccctgggggcagatggtggatttttcagtcgcgggcctggtgctggcgattgtggtggccgtcatcaagctggtgctgggggtgctgattatcgccctgtttgaaaacagcatggcgcgcctgcggttcctgaaaacgtcccgggtgacctgggccggttttggcttcgcgtttttagctttcgtctccttcctggtggcgtaataagagagtgttttgtatgtctgaagaaaaaatcggtcaacagtatctgaacgcgctgcaccaggcgtttcccggtgtggttctggacgaagcgtggcagacccgcgaccagataaccatcaccgtgaaaaccaact

SEQ ID NO: 21 - DNA, 2375 bp, Artificial sequence
  Synthesized hycC
gtcaacagcccgaaagcccctccggcggcaccggccccggcccgggtcagcaccctgctggactgggtgcccggcattcgcgccatcgccgtgaaatgcgacctgtgcagctttgacgagcaggggcccgcctgtgtgcgcacctgcccgaccaaagcgctgcgcttagtggacaaccgggatatcgccctgtccagtaaacgtaaacgcgagatggcgatgaatatcgcttttgacgatttgtccatgttcaccggccagagcgaggcaaccaaatgaatgcactgacgctactttactgggcgctgtccgggtttgtcctgagtgcggtgctcgccggggttttctataaagccaaagccgtaagcggctttctggccgggcttggcggcgctgtcagtagcgcactggcgctgggcgcgggtgtacaggcgttgatgagcggtgccaccggtgaaatgggcctgcgcttttttcaccagaccttacaactgacccccatgaacggcctgtggctgattgccgtctgcctgccggcgctgtttgtcagcctgtttaacatcagctggcaccagcacgccggggtgaaggccaacggcctgctgatcaacctgctgctggcggcagcggtaatggcgctgaccgccggtaacatcgccaccctggtgctgatggcggaaatcatggccctgtgcgcggcgttcctgaccggttgtgctcagtccggcaaactgtggtttgccctgggccgcctgggcacagtgctgctggttatcgcctgcctgctactggaaaaacgctacggcaccctggactacgccgccctgcatgaggcgaccgcgttacagccgctgggctccggtatctggttactggggctggtgggctttggcctgctggccgggatcatcccgctgcatggctgggtgccccaggctcacgctaacgcctctgccccggcggcggcgctgttttcagccgtggtgctgaaagtgggtctgttcgggatcctcactttttcgcttatcgacgcggcgctgccgctgtggtggggcattgtggtgatggtgctggggatgctgaccgcgtttattggcggcctgtatgccctgatggagcacaacatcaaccgcctgctggcgtaccactccctggaaaacatcggcattattctgctcggcctcggggcgggcttaaccggcctttccctgaatgagccggggcttatcgcccttggcctgaccggtggcctgttccacctggtgaaccacagcctgtttaaatccaccctgttcctcggggccggggccgtgtggttccgtaccggctaccgcgacattgaaaagctgggcggtatcggcaaacgcatgccgcttatttcgctggcgatgctggtagggctgatgtccatggccgcgctgccaccgctgaacggctttgccggtgagtgggtgatttaccagtccttcttccgcctgggtgagcagccgctgtttgccgcccgcctgctggggccgctgctggcggtagggctggctatcaccggtgcgctggcagtgatgtgtatggcgaaagtctacggggtgaccttcctcggcgcgccgcgcacccgggaggccagtgaggccaaaccggccccgctgctgatgaacctcagcgtggcggcactggccctgtgctgtgtagcttccggtgtggctgccccctggatcctgccgctgctgcgtaacgtggtgccgctgccgctggtttctgcccacagtgtggtgtcccagccgctgatgacgctgctgctgattggctgcccgctgctgccgttcctggtgatggtgttcttcaaaggcgatcgcctggtgtcccgctctcgcggtgcggcctgggtgtgtggttacgaccacgagcagtcgatggttatcaccgccacaggttttgcccagccggtaaaagcggcgtttgccccgctgttaaacctgcgtaaccggctgaacccggtgcagtgggtgccgggctggcagagtgaatgcctggcgggctcggtgcgccgtctggcctttattgaactggcgattctgctggtgattgtggtttcccggggagcataaaaacaatgactgtgattcttggtttgattcaggcgctggtgctgtttgccattgcgccgctgatatccggcattacccgcgtagcccgcgcccggatgcataaccgtcaggggccgggggtgcttcaggagtaccgggatctgttcaagctgctgggccgccagagcgtggccccggcggcctctggctgggtcttccgcctgatgccgtttgtgatggtgggcgttatgctaaccatcgccagcgccctgccgctgattactgtcgccgctc

SEQ ID NO: 22 - DNA, 2222 bp, Artificial sequence
  Synthesized hycE
gctggtggtgttgcagatgtttgtcggggtgttcctgccctgggggcagatggtggatttttcagtcgcgggcctggtgctggcgattgtggtggccgtcatcaagctggtgctgggggtgctgattatcgccctgtttgaaaacagcatggcgcgcctgcggttcctgaaaacgtcccgggtgacctgggccggttttggcttcgcgtttttagctttcgtctccttcctggtggcgtaataagagagtgttttgtatgtctgaagaaaaaatcggtcaacagtatctgaacgcgctgcaccaggcgtttcccggtgtggttctggacgaagcgtggcagacccgcgaccagataaccatcaccgtgaaaaccaactacctgccggaagtggtggagtttctctactaccagcaggggggctggctgtcggtactgttcgggaacgacgaacgcccgctgtgcggccattatgcggtctactatgtgctctccatggagaagggcaccaaatgctggatcaccgtgcgtgttgaagtggacgccaacaccctggaatacccgtctgtcacgccgcgcgtaccggcggctgtctggggcgagcgcgaagtgcgcgacatgtacggcctgcgcgccgttggcctgccggatgagcgccgcctggtgctgcccgatgactggccggacgatctctacccgctgcgcaaagacagcatggactaccgccagcgcccggcaccgaccaccgacgcagaaacctacgagttcatcaacgagctgggcaccaaaaagaacaacgtggtgccgattggcccgctgcatgtgacctctgacgaaccgggccacttccgcctgtttgttgacggtgaaaacatcattgatgcggactaccggctgttttatgtccaccgcggcatggagaaactggcagaaacccgcatgggttacaacgaggtgaccttcctctctgaccgggtgtgcggtatttgcgggtttgcccacagcaccgcgtataccacctcggtggaaaatgccatgggcatccaggtgccggaacgggcccagatgatccgcgccattctgctggaagtggaacgcctgcacagccacctgctcaacctggggctggcctgccactttgtgggctttgactccggctttatgcagttcttccgggtgcgtgagcagtccatgaaaatggcggaaatcctcaccggtgcccgtaaaacctacggcctgaacctgattggcggtatccgccgcgatatgctcaaagacgacatggtccagacccgccagctggcccagcagatgcgcaaagaagtccaggagctggtggatattctgatgagcaccaccaatatcgagcagcgcaccgtgggcattggccgcctggatccgcaaatcgcccgtgacttcagcaacgtcggcccgatggtgcgcgccagcggccacgcccgcgacgcccgctacgatcatgagtttgtcggttacggcctgctgccgatggagatccacagcgaacagggctgtgacgttatctcgcgcctgaaagtgcgcattaacgaggtgttcacctccctgaatatgatcgactacggcctggataacctgccgggcggcccgctggcggtggaaggctttacctatattccgcaccgtttcgccctcggctttgccgaagcgccgcgcggcgacgatatccactggagcatgaccggcgacaaccagaagctgttccgctggcgctgccgtgcggccacctacgccaactggccgaccctgcgctatatgctgcgcggtaacaccgtgtctgacgcgccgctgattatcggcagtctggatccttgctactcctgtactgaccgtatgaccgtggtcgacgtgcgcaagaagaagagcaaagtggtgccgtacaaagagcttgagcgctacagcattgaacgcaagaactcgccgctgaaataagccgttacccccccgccgggcggcggggggttgaggaagagcaatgtttacttttattaaaaaagtgctgaaaaccggggtagtcactgaaaactacccgctgacccccattgcggtggataaaaatttccgcggcaagccggagcataaccccagccagtgtgtgggctgtgcggcctgcgtgaacgcctgcccgtctaacgcgctgagcgtggaaaccaacctcgccgggcaaaccctcgactggcagtttaa

SEQ ID NO: 23 - DNA, 705 bp, Artificial sequence
  Synthesized hycF
cgctacagcattgaacgcaagaactcgccgctgaaataagccgttacccccccgccgggcggcggggggttgaggaagagcaatgtttacttttattaaaaaagtgctgaaaaccggggtagtcactgaaaactacccgctgacccccattgcggtggataaaaatttccgcggcaagccggagcataaccccagccagtgtgtgggctgtgcggcctgcgtgaacgcctgcccgtctaacgcgctgagcgtggaaaccaacctcgccgggcaaaccctcgactggcagtttaacctcgggcgctgcatcttctgcggccgctgtgaggaagtctgcccgacggcggccattaaactctctcagcagtatgagctggcggtgtggaagaaagaggatttcctgcagaaatccagcttccccctgtgccactgccgcagctgccagcgcccgtttgcggtgcaaaaagagattgattacgccatcgcgattttgcagcacaacggcgaccagcgcgccgaacagcaccgggaaagttttgagacctgcccggagtgcaaacgccagcaggggctggtctcctctgaccgtattgatttaacccgccatatgaaagaggccagctgatgagcaacttaatcggcccgcgcgacgccaacgggatccccgtacctatgacggtggatgaatccatcgccagcatgaaa

SEQ ID NO: 24 - DNA, 995 bp, Artificial sequence
  Synthesized hycG
cagcgcgccgaacagcaccgggaaagttttgagacctgcccggagtgcaaacgccagcaggggctggtctcctctgaccgtattgatttaacccgccatatgaaagaggccagctgatgagcaacttaatcggcccgcgcgacgccaacgggatccccgtacctatgacggtggatgaatccatcgccagcatgaaagcatcgctgctgaaaaatattaaacgctccgcctatgtctaccgggtggactgcggcggctgtaacggctgcgaaattgaaatcttcgccaccctgtcgccgctgtttgacgccgagcgcttcgggatcaaggtggtgccctctccgcgccatgcggatattttgctgtttaccggtgcggtgacccgcgccatgcgctccccggcactgcgtgcctggcagtctgcgccggatccgaagatctgcatctcttacggtgcctgcggcaacagcggcggtatcttccacgacctgtactgcgtgtggggcggtacggacaagatagtcccggtggatgtctatattccgggttgcccgccaaccccggccgccaccctgtatggctttgccatggctctgggcctgctggagcagaaaatccacgcccgggacgccagcgaactggatacccagcctgcgcagatcctgcacccggatatggtgcagcccctgcgggtgcgcattgaccgcacggcgcgcaaactggcgggctaccgctatggccgccagattgccgatgactatatgaaccatctggcccagggggaagccggtattgcccgctggctggaggcggaaaacgacccgcggctgagcgaaattgtcaccaatcttaaccagctggttgagcaggagcgtatctgatgagcgagtcggtggtgttcagccagctgagccgtaaatttgtcgataatgataacaaggcgccggatcaggcccagcaagttatctactatagtctggcgattggtcaccacc

SEQ ID NO: 25 - DNA, 791 bp, Artificial sequence
  Synthesized hycB
ggcctgatcctgattcgtgtgtcgaaccctgtcgaaaatgacactgtccggcatgttttcgtcaaaaatgactattcctgaggtaatacccggtgaaccgttttgtaattgccgacccgaagctctgtatggggtgccacacctgtgaggccgcctgctcagagactcaccggcagcatgggttgcagtccatgccgcgtttacgcgtgatgctcaatgcccatgagtccgcaccgcagctctgccaccattgtgaagatgcgccctgtgccactgtctgcccggtgaacgccattaaccgtgtggatggcgccgtacagctgaatgaaagcctgtgcgtaagctgcaaactgtgcggcatcgcctgcccgtttggcgctatcgaattttccggcagccgcccgctgaatattccggccaacgtcaacagcccgaaagcccctccggcggcaccggccccggcccgggtcagcaccctgctggactgggtgcccggcattcgcgccatcgccgtgaaatgcgacctgtgcagctttgacgagcaggggcccgcctgtgtgcgcacctgcccgaccaaagcgctgcgcttagtggacaaccgggatatcgccctgtccagtaaacgtaaacgcgagatggcgatgaatatcgcttttgacgatttgtccatgttcaccggccagagcgaggcaaccaaatgaatgcactgacgctactttactgggcgctgtccgggtttgtcctgagtgcggtgctcgccggggttttctataaagccaaagccgtaagcg

SEQ ID NO: 26 - DNA, 1907 bp, Artificial sequence
  Synthesized hoxH
gtcccgggcgccaaggccgtcgttccattccatcccgatatcccgaggatcaccaccaaggtctacccttgtcatgaggtggtcaagatggattatttcattccgggttgtcccccggatggagatgccatcttcaaggtgctggacgatctggtgaatggacggccattcgatttgccgagctcgatcaatcgctacgattgattaacggagggtttgttatgagcagaaaactggttatcgacccggtgacccgcatcgagggtcacggcaaggtggtggtgcacctggatgacgacaacaaggtcgtcgacgcaaagctgcacgtcgtggagttccggggctttgaaaagttcgttcagggccatcccttctgggaggcgccgatgttcctgcagcgcatctgcggcatctgtttcgtcagtcaccatctgtgcggggccaaggcgctggatgacatggtcggcgtcggcttgaagtccggcatccatgtcaccccgacggcggagaaaatgcgccggctcggccactacgcgcagatgctccagtcccatacgaccgcctatttctacctgatcgtgccggagatgctgtttggcatggacgcgccgcccgcacagcgcaacgtgctcggcctgatcgaggccaatcccgacttggtgaagcgcgtcgtgatgttgcgcaaatggggacaggaagtcatcaaggcggtgttcggcaagaagatgcacggcatcaattcggtgccgggaggcgtcaacaacaacctgagcatcgccgagcgcgaccgtttcctgaacggggaggagggccttctgtcggtggatcaggtcatcgattacgcgcaggatggcctgcgcctgttctacgacttccatcagaaacaccgggcgcaggtcgatagtttcgcggacgtgcccgcgctcagcatgtgcctggtcggcgacgacgacaacgtggactactaccacggcaggctgaggatcatcgacgacgacaagcacatcgtccgtgaattcgactatcacgactatctggatcatttctccgaagcggtggaggaatggagctacatgaagttcccctacctcaaggagcttgggagagagcagggatcggtgcgcgtggggccgcttggccgcatgaacgtgacgaagtcgctcccgacaccgctcgcgcaggaggcgctggaacggttccacgcctacacgaaggggcggacgaacaacatgacgctgcatactaactgggcacgggccatcgagatcctccacgccgcggaggtggtcaaagaactgctgcatgacccggacctgcagaaggatcagctggtgttgacaccgccccccaatgcgtggacaggtgaaggcgtcggcgtggtcgaagcgccacgcggtaccttgcttcaccattatcgtgccgatgagcgcggcaatatcacgttcgccaacctggtcgtcgccaccacccagaacaaccaggtcatgaatcgcacggtgcgcagcgtcgccgaggactacctgggcggacatggcgaaatcaccgagggcatgatgaatgccatcgaggtgggtattcgcgcctacgatccatgcctgagctgcgcgacacacgccctggggcagatgccgctggtggtctcggtctttgacgcggcggggcgcctgatcgatgaacgcgcccgctgagtttccctatgtgacccttgccgatttcgatgatccgtcgacgctgatctacggaatcggcaacgtgggtcggcaggacgacggattggggtgggctttcatcgaccgactggaagcggagtccctctgctccggggcggaggttcagcggcattatcagcttcacctcgaggatgccgacctgatcagccgcaagagaaaggtgttgttcatcgatgc

SEQ ID NO: 27 - DNA, 818 bp, Artificial sequence
  Synthesized hoxY
attctcgagaaacgggtcggttatgacgatcccatcggacgacgcaagtacgaaatccagtcggtgcgcgcacgcgcgctggaaggagaagacaaatgagagccccccacaaagacgagattgcgtcgcacgaattgcctgcgacgccgatggatccggcgctggccgcgaaccgtgaaggcaagatcaaggtggccacgatcggtctgtgcggctgctgggggtgtaccttgtcctttctcgacatggacgagcggctcctgccgctgttggagaaagtcaccctcctccgctcgtcgctgaccgatatcaaacgaattccggagcgctgtgcgatcggcttcgtggaaggtggcgtctcgagcgaggagaacatcgaaacgctggagcactttcgcgagaactgcgacatcctgatttcggtcggggcgtgcgcggtgtggggcggtgtgccggcgatgcgcaacgtcttcgagctgaaagattgtctggcagaggcttatgtcaactcggcgactgccgtcccgggcgccaaggccgtcgttccattccatcccgatatcccgaggatcaccaccaaggtctacccttgtcatgaggtggtcaagatggattatttcattccgggttgtcccccggatggagatgccatcttcaaggtgctggacgatctggtgaatggacggccattcgatttgccgagctcgatcaatcgctacgattgattaacggagggtttgttatgagcagaaaactggttatcgacccggtgacccgcatcgagggtcacggcaaggtggtggtgcacctggatgacg

SEQ ID NO: 28 - DNA, 2351 bp, Artificial sequence
  Synthesized hoxF
ggcgctggcacaagcttgcacagacttgcccgccaagcggaagcagccttgccacatcggccgacccaatggcaatgccgctgccacccgccggatggccgttctggaaacggcttgagcgacgtcaagaatttcctttctcgacaagcacttagccgggcctcctggtggtttcccttaggccctgcgaaattggcgcacatcctgcgttccacctgcgcatcgaagtgacgcaccaagcaaggggcgaacattagtaaggaggagacaacatggatagtcgtatcacgacaatactcgagcgctaccgctcagaccgtacacggctgatcgacatactttgggatgttcagcatgagtatgggcacattcccgatgcggtactgccgcaactgggggctgggttgaagctgtccccgctggacattcgcgaaacggcgtcgttctaccactttttccttgacaagccgtcgggcaagtatcggatttacttgtgcaattccgtgattgccaagatcaacggctatcaggcggtgcgtgaggcgctcgaacgcgagactgggattcgcttcggcgaaaccgacccgaatgggatgtttggcctgttcgacaccccctgtatcggactcagcgatcaggaaccggcgatgctgatcgataaggtggtattcacccgcctgcgacccggaaagatcacggacatcatcgcgcagttgaaacaaggacgatcgccggccgagatcgcgaacccggccggtttgcccagtcaggacatcgcctatgtcgatgccatggtcgagtccaatgtccgcaccaaggggccggtgttcttccgtggccggacggatttgagatctttgctcgaccaatgcctgctgctcaagcccgaacaagtgattgagaccatcgtcgactccaggctgcgcggacgtggcggcgcagggttctcgaccgggctgaagtggcggctgtgtcgggatgccgaaagcgagcagaagtatgtaatctgcaacgccgacgaaggtgagcccggcacgttcaaggatagggtcctcctgacacgcgctcccaagaaggttttcgtcggaatggttatcgccgcgtatgcgatcggctgccgcaagggtatcgtctatctgcggggggaatacttctacctcaaggattatctggagcgacagcttcaggaacttcgggaggacgggttgctggggcgcgctatcggtggccgggcgggctttgatttcgatatccgtattcagatgggggccggcgcttatatctgcggcgacgaatcggcgctcatcgagtcctgcgaggggaaacggggcacgccacgggtgaaacctccgttcccggtgcagcaagggtatctgggcaagcccaccagcgtcaacaacgttgagacctttgccgccgtgtcgcggatcatggaggaaggcgcggactggttccgggcgatgggaacgccagactcggccggcacccggctgctgagcgtggctggcgattgcagcaagcctggcatctacgaggtggaatggggggtcaccctcaacgaagtgctggcgatggtcggagcgcgggacgcgcgggccgtccagatcagcggtccttccggtgaatgcgtgtcggtggcaaaggacggtgagcgcaagctcgcgtacgaagatctttcgtgcaatggcgccttcaccattttcaactgcaagcgcgacctgctggaaatcgtgcgtgaccacatgcagttcttcgtcgaagagtcctgcggcatttgtgtgccatgtcgcgccggcaacgttgatctgcaccggaaggtcgaatgggtcatcgcgggcaaggcctgccagaaggatctggacgatatggtcagttggggagcgctggtgcggaggaccagtcgatgtggccttggggccacatcgcccaagcccatcctgacgacgctggagaaattccccgagatctatcagaacaagctggtgaggcacgagggcccgctgctgccatcgttcgatctcgataccgccttgggcgggtatgagaaggcgctgaaggatctggaagaggtgacaagatgagcattcaaattacgatcgacggcaagacgctcacgaccgaggaaggacgaacgctggtggatgttgccgcagagaacggcgtttacatcccgacgctgtgctacctcaaggacaagccctgcctcggcacctgccgggtgtgttcggtcaaggtgaatggcaatgtcgccgcggcatgtacggtgcgggtctcgaagggcctgaatgtcgaggtcaacgaccccgaattggtcgacatgcgcaaggcgctggtcgaattcctgttcgcgg

SEQ ID NO: 29 - DNA, 917 bp, Artificial sequenence
  Synthesized hoxU
cagaacaagctggtgaggcacgagggcccgctgctgccatcgttcgatctcgataccgccttgggcgggtatgagaaggcgctgaaggatctggaagaggtgacaagatgagcattcaaattacgatcgacggcaagacgctcacgaccgaggaaggacgaacgctggtggatgttgccgcagagaacggcgtttacatcccgacgctgtgctacctcaaggacaagccctgcctcggcacctgccgggtgtgttcggtcaaggtgaatggcaatgtcgccgcggcatgtacggtgcgggtctcgaagggcctgaatgtcgaggtcaacgaccccgaattggtcgacatgcgcaaggcgctggtcgaattcctgttcgcggaaggcaaccacaactgcccgagttgcgagaagagcggccgttgccagttgcaggcggtcggctacgaggtggacatgatggtctcgcgctttccgtaccggttcccggtccgcgtggtggaccacgcgtccgaaaagatctggctcgagcgggatcggtgcatcttctgtcagcgctgtgtcgagttcatccgcgacaaggcaagcggccggaagatcttcagcatcagccatcggggtcccgagtcgcgcatcgagatcgatgccgaactggcgaacgccatgccgccggagcaagtcaaagaggcggttgcgatctgcccggtgggcaccattctcgagaaacgggtcggttatgacgatcccatcggacgacgcaagtacgaaatccagtcggtgcgcgcacgcgcgctggaaggagaagacaaatgagagccccccacaaagacgagattgcgtcgcacgaattgcctgcgacgccgatggatccggcgctggccgcgaaccgtgaaggcaagatcaaggtggccacgatcg

SEQ ID NO: 30 - DNA, 1918 bp, Artificial sequence
  Synthesized erg13
taagcttcgtatatacaatagaaaaattttccatacaatattacataatactatagagtaatcatacccacaatctccatggctgcggtatcgtgtcgaaaatgaaccgggctattaattatacgagtgtgttgaaagtaggctccattcggcctcatcgggcggaactgtttgggtgcctcgccaattttcgtagccaaattactataaaaggtgcagcatgaaactctcaactaaactttgttggtgtggtattaaaggaagacttaggccgcaaaagcaacaacaattacacaatacaaacttgcaaatgactgaactaaaaaaacaaaagaccgctgaacaaaaaaccagacctcaaaatgtcggtattaaaggtatccaaatttacatcccaactcaatgtgtcaaccaatctgagctagagaaatttgatggcgtttctcaaggtaaatacacaattggtctgggccaaaccaacatgtcttttgtcaatgacagagaagatatctactcgatgtccctaactgttttgtctaagttgatcaagagttacaacatcgacaccaacaaaattggtagattagaagtcggtactgaaactctgattgacaagtccaagtctgtcaagtctgtcttgatgcaattgtttggtgaaaacactgacgtcgaaggtattgacacgcttaatgcctgttacggtggtaccaacgcgttgttcaactctttgaactggattgaatctaacgcatgggatggtagagacgccattgtagtttgcggtgatattgccatctacgataagggtgccgcaagaccaaccggtggtgccggtactgttgctatgtggatcggtcctgatgctccaattgtatttgactctgtaagagcttcttacatggaacacgcctacgatttttacaagccagatttcaccagcgaatatccttacgtcgatggtcatttttcattaacttgttacgtcaaggctcttgatcaagtttacaagagttattccaagaaggctatttctaaagggttggttagcgatcccgctggttcggatgctttgaacgttttgaaatatttcgactacaacgttttccatgttccaacctgtaaattggtcacaaaatcatacggtagattactatataacgatttcagagccaatcctcaattgttcccagaagttgacgccgaattagctactcgcgattatgacgaatctttaaccgataagaacattgaaaaaacttttgttaatgttgctaagccattccacaaagagagagttgcccaatctttgattgttccaacaaacacaggtaacatgtacaccgcatctgtttatgccgcctttgcatctctattaaactatgttggatctgacgacttacaaggcaagcgtgttggtttattttcttacggttccggtttagctgcatctctatattcttgcaaaattgttggtgacgtccaacatattatcaaggaattagatattactaacaaattagccaagagaatcaccgaaactccaaaggattacgaagctgccatcgaattgagagaaaatgcccatttgaagaagaacttcaaacctcaaggttccattgagcatttgcaaagtggtgtttactacttgaccaacatcgatgacaaatttagaagatcttacgatgttaaaaaataatcttcccccatcgattgcatcttgctgaacccccttcataaatgctttatttttttggcagcctgctttttttagctctcatttaatagagtagttttttaatctatatactaggaaaactctttatttaataacaatgatatatatatatattttttttataaagaattgtatatctatatttataacacaataaatctaatctcaacttttttctttaaa

SEQ ID NO: 31 - DNA, 1665 bp, Artificial sequence
  Synthesized mvaA
ttatacaaagaaatacactaaatttaaaaatgatttgacgtttcttgtaagcgttttaatttaccttgctttattttaaagtcaaaacgacaattattcaattgaattttaagacgaataacgttattttatggtaaaaatagtagtgattgaaatgttaaaaaattatgtgaagtaaaaggagtatgtccatgcaaagtttagataagaatttccgacatttatctcgtcaacaaaagttacaacaattggtagataagcaatggttatcagaagagcaattcaacattttattgaatcatccattaattgatgaggaagtagcaaatagtttaattgaaaatgtcatcgcgcaaggtgcattacccgttggattattaccgaatatcattgtggacgataaagcatatgttgtacctatgatggtggaagagccttcagttgtcgctgcagctagttatggtgcaaagctagtgaatcagactggtggatttaaaacggtatcttctgaacgtattatgataggtcaaatcgtctttgatggcgttgacgatactgaaaaattatcagcagacattaaagctttagagaagcaaattcatcaaattgcggatgaggcatatccttctattaaagcgcgtggtggtggttaccaacgtatagctattgatacatttcctgagcaacagttactatctttaaaagtatttgttgatacgaaagatgctatgggcgctaatatgcttaatacgattttagaggctataactgcatttttaaaaaatgaatttccgcaaagcgacattttaatgagtattttatccaatcatgcaacagcgtccgttgttaaagttcaaggcgaaattgatgttaaagatttagcaaggggcgagagaactggagaagaggttgccaaacgaatggaacgtgcttctgtattggcacaagttgatattcatcgtgctgcaacacataataaaggtgttatgaatggcatacatgccgttgttttagcaacaggaaatgatacgcgtggtgcagaagcaagtgcgcatgcatacgcgagtaaagatggtcaatatcgtggtattgcaacatggagatacgatcaagaacgtcaacgtttaattggtaccatagaggtgcctatgacattggcaatcgttggcggtggtacaaaagtattaccaatagctaaagcatcattagagctactaaatgtagagtcagcgcaagaattaggtcatgtagttgctgccgttggtttagcacaaaactttgcagcatgtcgcgcgctcgtttctgaaggtatccagcaaggccatatgagcttgcaatataaatctttagctattgttgtaggtgcaaaaggtgatgaaattgcgcaagtagctgaagcattgaagcaagaacctcgtgcgaatacacaagttgctgaacgcattttacaagatttaagaagccaacaataatcaaatgcgaatagcgattttaaagaaaagtttaagtcagtccatcgtagcgcttaaataaattaatatgaacaaaaaataaaaaacttctatcacacgcatacaggaatttgaagcgttcgtgatagaagttttttgattagtaaatatagttaaagttacgtgctgcctttttgcttagtacacgtgtatt

SEQ ID NO: 32 - DNA, 1665 bp, Artificial sequence
  Synthesized mvaA
cttatacaaagaaatacactaaatttaaaaatgatttgacgtttcttgtaagcgttttaatttaccttgctttattttatagttaaaacaacaattattcaattgaattttaggacgaataacgttattttatggtaaaaatagtagtgattgaaatgttaaaaattatgtgaagtaaaaggagtatgtccatgcaaagtttagataagaatttccgacatttatctcgtcaacaaaagttacaacaattggtagataagcaatggttatcagaagagcaattcaacattttattgaatcatccattaattgatgaggaagtagcaaatagtttaattgaaaatgtcatcgcgcaaggtgcattacccgttggattattaccgaatatcattgtggacgataaggcatatgttgtacctatgatggtggaagagccttcagttgtcgctgcagctagttatggtgcaaagctagtgaatcagactggcggatttaaaacggtatcttctgaacgtattatgataggtcaaatcgtctttgatggcgttgacgatactgaaaaattatcagcagaaattaaagctttagaaaatcaaattcataaaattgcggatgaggcatatccttctattaaagcgcgtggtggtggttaccaacgtatagctattgatacatttcctgagcaacagttactatctttaaaagtatttgttgatacgaaagatgctatgggcgctaatatgcttaatacgattttagaggccataactgcatttttaaaaaatgaatttccgcaaagcgacattttaatgagtattttatccaatcatgcaacagcgtccgttgttaaagttcaaggcgaaattgacgttaaagatttagcaaggggtgagagaactggagaagaggttgccaaacgaatggaacgtgcttctgtattggcacaagttgatattcatcgtgctgcaacacataataaaggtgttatgaatggcatacatgccgttgttttagcaacaggaaatgatacgcgtggtgcagaagcaagtgcgcatgcatacgcaagtaaagatggtcaatatcgtggtattgcaacatggagatacgatcaagaacgtcaacgtttaattggtacaatagaagtgcctatgacattggcaatcgttggcggtggtacaaaagtattaccaattgctaaagcttctttagaattgctaaatgtagattcagcacaagaattaggtcatgtagttgctgccgttggtttagcgcaaaactttgcagcatgtcgcgcgctcgtttccgaaggtatccagcaaggccatatgagcttgcaatataaatctttagctattgttgtaggtgcaaaaggtgatgaaattgcgcaagtagctgaagcattgaagcaagaaccccgtgcgaatacacaagtagctgaacgcattttacaagatttaagaagccaacaataatcaaatgcgaatagcgattttaaagaaaagtttaagtcagtccatcgtagcgcttagataaattaatatgaacaaaaaataaaaacttctatcacacgcatacaggaatttgaagcgttcgtgatagaagtttttagattagtaaatatagttaaagttacgtgctgcctttttgcttagtatacgtgtattg

SEQ ID NO: 33 - DNA, 4115 bp, Artificial sequence
  Synthesized mvaA
ggactaagggaaagaaaagccttctggagaagagttggaagacctcagcgtagtgcgaagaaaacgtagtgcgagtctctacgcccgctcgtttaaaactctatagaacgtccgaaagaaaggtccgtactagctataggatctctactttacctattattatcgctaggtgatagcttataaaacaattcatcggtttcttcctcatttttcctgtatcttttcggctatgaaaagctgttgttcgataaatccaggcaaagatttttgaaagtttaaaagatctaattttcaaaggcattcctttaaaaaagacaacttgaaagagctatattcgtcttcggttttttgatttttattaacttatttttttcttctttctacccaattctagtcaggaaaagactaagggctggaacatagtgtatcattgtctaattgttgatacaaagtagataaatacataaaacaagcatgccgccgctattcaagggactgaaacagatggcaaagccaattgcctatgtttcaagattttcggcgaaacgaccaattcatataatacttttttctctaatcatatccgcattcgcttatctatccgtcattcagtattacttcaatggttggcaactagattcaaatagtgtttttgaaactgctccaaataaagactccaacactctatttcaagaatgttcccattactacagagattcctctctagatggttgggtatcaatcaccgcgcatgaagctagtgagttaccagccccacaccattactatctattaaacctgaacttcaatagtcctaatgaaactgactccattccagaactagctaacacggtttttgagaaagataatacaaaatatattctgcaagaagatctcagtgtttccaaagaaatttcttctactgatggaacgaaatggaggttaagaagtgacagaaaaagtcttttcgacgtaaagacgttagcatattctctctacgatgtattttcagaaaatgtaacccaagcagacccgtttgacgtccttattatggttactgcctacctaatgatgttctacaccatattcggcctcttcaatgacatgaggaagaccgggtcaaatttttggttgagcgcctctacagtggtcaattctgcatcatcacttttcttagcattgtatgtcacccaatgtattctaggcaaagaagtttccgcattaactctttttgaaggtttgcctttcattgtagttgttgttggtttcaagcacaaaatcaagattgcccagtatgccctggagaaatttgaaagagtcggtttatctaaaaggattactaccgatgaaatcgtttttgaatccgtgagcgaagagggtggtcgtttgattcaagaccatttgctttgtatttttgcctttatcggatgctctatgtatgctcaccaattgaagactttgacaaacttctgcatattatcagcatttatcctaatttttgaattgattttaactcctacattttattctgctatcttagcgcttagactggaaatgaatgttatccacagatctactattatcaagcaaacattagaagaagacggtgttgttccatctacagcaagaatcatttctaaagcagaaaagaaatccgtatcttctttcttaaatctcagtgtggttgtcattatcatgaaactctctgtcatactgttgtttgtcttcatcaacttttataactttggtgcaaattgggtcaatgatgccttcaattcattgtacttcgataaggaacgtgtttctctaccagattttattacctcgaatgcctctgaaaactttaaagagcaagctattgttagtgtcaccccattattatattacaaacccattaagtcctaccaacgcattgaggatatggttcttctattgcttcgtaatgtcagtgttgccattcgtgataggttcgtcagtaaattagttctttccgccttagtatgcagtgctgtcatcaatgtgtatttattgaatgctgctagaattcataccagttatactgcagaccaattggtgaaaactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaaaggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgttgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccgcatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaaacttagtcatacgtcattggtattctcttgaaaaagaagcacaacagcaccatgtgttacgtaaaatatttactttatagtttgtacgtcataatttcttccatattacaagttcgtgcatatatagaaagaattctgttgttgtaattgtcataactattgagctttacctgaaaattcaacgaaaaaaactcaaaaaccacatgcttctcttgagtcatgcggttcctttcccttatgagtgaaaatcttccttttttagctatgtgcgccatccgataaatgtaggagcaatgaagcggaaagtcaattttttcatacagtatacattaatatctagtgtgtattgactgtgatcgggaagatcttcagggttgaattacaacatggacacgcagattgcaataactggcgttgccgtcgggaaagaaatcaataatgataattcaaagaccgaccaaaaagtttctttgccg

SEQ ID NO: 34 - DNA, 1732 bp, Artificial sequence
  Synthesized erg12
gtgtattttatcggttgtaattgtactgacaattttcgggcctcgtttggctgtcgcactgaaatcttcgacagggtatcgaagaacgatacaacgatatgcctataaaggttagttagccatgatattacttcctggttagctggtgcgcttcttagtcctaacttgcaaatttatatctacgtatagaaaactgtcaatatgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcataagctaatttgcgataggcattatttattagttgtttttaatcttaactgtgtatgaagttttatgtaataaagatagaaagagaaacaaaaaaaaatttttcgtagtatcaattcagctttcgaagacagaatgaaatttaagcagaccatagtatccttgatacattgactcataatcagttaaatagccattcaatcc

SEQ ID NO: 35 - DNA, 1762 bp, Artificial sequence
  Synthesized erg8
aaattgtatacaggaatatgtagatagtaataattattgcgatgcgtttttctatacgagatctaaaaggaaaaaagttcggttttaaggcggagttaacgtcatgtcgaatggaaagaaagattatataaagaatggaaaaacaaaaaaggtgaaaaaacatcttggaaggcctcataataaatcaatcggctgcctcgagaaatgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaataacttaaggtagataatagtggtccatgtgacatctttataaatgtgaagtttgaagtgacagcgcttaacatctaaccattcatcttccgatagtacttgaaattgttcctttcggcggcatgataaaattcttttaatgggtacaagctatacatactaggatgaggatggtactgagaacgataaataaactttctagata

SEQ ID NO: 36 - DNA, 1549 bp, Artificial sequence
  Synthesized mvd1
cgcccttctgataaccttacttttaattttggcctgattcttgtgaaacttttccacgatgccgaaatcgccgaaatggctcgtttagccttttctcgaagattgatatataaagagcatccatcgtgctggtaatggtgatgcaagattgaaaattcccagacagtgagcaccagcacaatgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataagatcaattcgatatgtaacatttttcttttcttttcttttcctttttttacaatagctaatttacgtttccctacggtattggtcggaacgaccaagcttcaatttataaatatcttaattttaacagcagttaccacttgaatgagaaactttttgatgcctactcttttgcttttt

SEQ ID NO: 37 - DNA, 713 bp, Artificial sequence
  Synthesized idi
tgtcgataaacgctcacttggttaatcatttcactcttcaattatctataatgatgagtgatcagaattacatgtgagaaattatgcaaacggaacacgtcattttattgaatgcacagggagttcccacgggtacgctggaaaagtatgccgcacacacggcagacacccgcttacatctcgcgttctccagttggctgtttaatgccaaaggacaattattagttacccgccgcgcactgagcaaaaaagcatggcctggcgtgtggactaactcggtttgtgggcacccacaactgggagaaagcaacgaagacgcagtgatccgccgttgccgttatgagcttggcgtggaaattacgcctcctgaatctatctatcctgactttcgctaccgcgccaccgatccgagtggcattgtggaaaatgaagtgtgtccggtatttgccgcacgcaccactagtgcgttacagatcaatgatgatgaagtgatggattatcaatggtgtgatttagcagatgtattacacggtattgatgccacgccgtgggcgttcagtccgtggatggtgatgcaggcgacaaatcgcgaagccagaaaacgattatctgcatttacccagcttaaataaaaaaaccccgacatttgccggggttgtgagcataacgtaatgcttattttaccggacgcatcgccgggaacagaataacgt

SEQ ID NO: 38 - DNA, 1788 bp, Artificial sequence
  Synthesized ispS
atggcaactgaattattgtgcttgcaccgtccaatctcactgacacacaaacttttcagaaatcccttacctaaagtcatccaggccactcccttaactttgaaactcagatgttctgtaagcacagaaaacgtcagcttcacagaaacagaaacagaagccagacggtctgccaattatgaaccaaatagctgggattatgattatttgctgtcttcagacactgacgaatcgattgaggtatacaaagacaaggccaaaaagctggaggctgaggtgagaagagagattaacaatgaaaaggcagagtttttgactctgcttgaactgatagataatgtccaaaggttaggattgggttaccggttcgagagtgacataaggggagcccttgatagatttgtttcttcaggaggatttgatgctgttacaaaaactagccttcatggtactgctcttagcttcaggcttctcagacagcatggttttgaggtctctcaagaagcgttcagtggattcaaggatcaaaatggcaatttcttggaaaaccttaaggaggacatcaaggcaatactaagcctatatgaagcttcatttcttgcattagaaggagaaaatatcttggatgaggccaaggtgtttgcaatatcacatctaaaagagctcagcgaagaaaagattggaaaagagctggccgaacaggtgaatcatgcattggagcttccattgcatcgcaggacgcaaagactagaagctgtttggagcattgaagcataccgtaaaaaggaagatgcaaatcaagtactgctagaacttgctatattggactacaacatgattcaatcagtataccaaagagatcttcgcgagacatcaaggtggtggaggcgagtgggtcttgcaacaaagttgcattttgctagagacaggttaattgaaagcttttactgggcagttggagttgcgttcgagcctcaatacagtgattgccgtaattcagtagcaaaaatgttttcatttgtaacaatcattgatgatatctatgatgtttatggtactctggacgagttggagctatttacagatgctgttgagagatgggatgttaatgccatcaatgatcttccggattatatgaagctctgcttcctagctctctacaacactatcaatgagatagcttatgacaatctgaaggacaagggggaaaacattcttccatacctaacaaaagcgtgggcagatttatgcaatgcattcctacaagaagcaaaatggttgtacaataagtccacaccaacatttgatgactatttcggaaatgcatggaaatcatcctcagggcctcttcaactagtttttgcctactttgccgtggttcaaaacatcaagaaagaggaaattgaaaacttacaaaagtatcatgataccatcagtaggccttcccacatctttcgtctttgcaacgacctggcttcagcatcggctgagatagcgagaggtgaaacagcgaattctgtatcatgctacatgcgtacaaaaggcatttctgaggagcttgctactgaatccgtaatgaacttgatcgacgaaacctggaaaaagatgaacaaagaaaagcttggtggctctttgtttgcaaaaccttttgtcgaaacagctattaaccttgcacggcaatcccattgcacttatcataacggagatgcgcatacttcaccagacgagctaactaggaaacgtgtcctgtcagtaatcacagagcctattctaccctttgagagataa

SEQ ID NO: 39 - DNA, 1377 bp, Artificial sequence
  Synthesized erg20
aaatagaggaagcaacggcaggaaatatatataaacgcatgtcgaaactaatactttatgatagattgttcttctatcagttttcattttaactttaaaaactcaaccaacaggtattggactgacataggcacaataaactcaaaaatattacgtagaaatggcttcagaaaaagaaattaggagagagagattcttgaacgttttccctaaattagtagaggaattgaacgcatcgcttttggcttacggtatgcctaaggaagcatgtgactggtatgcccactcattgaactacaacactccaggcggtaagctaaatagaggtttgtccgttgtggacacgtatgctattctctccaacaagaccgttgaacaattggggcaagaagaatacgaaaaggttgccattctaggttggtgcattgagttgttgcaggcttacttcttggtcgccgatgatatgatggacaagtccattaccagaagaggccaaccatgttggtacaaggttcctgaagttggggaaattgccatcaatgacgcattcatgttagaggctgctatctacaagcttttgaaatctcacttcagaaacgaaaaatactacatagatatcaccgaattgttccatgaggtcaccttccaaaccgaattgggccaattgatggacttaatcactgcacctgaagacaaagtcgacttgagtaagttctccctaaagaagcactccttcatagttactttcaagactgcttactattctttctacttgcctgtcgcattggccatgtacgttgccggtatcacggatgaaaaggatttgaaacaagccagagatgtcttgattccattgggtgaatacttccaaattcaagatgactacttagactgcttcggtaccccagaacagatcggtaagatcggtacagatatccaagataacaaatgttcttgggtaatcaacaaggcattggaacttgcttccgcagaacaaagaaagactttagacgaaaattacggtaagaaggactcagtcgcagaagccaaatgcaaaaagattttcaatgacttgaaaattgaacagctataccacgaatatgaagagtctattgccaaggatttgaaggccaaaatttctcaggtcgatgagtctcgtggcttcaaagctgatgtcttaactgcgttcttgaacaaagtttacaagagaagcaaatagaactaacgctaatcgataaaacattagatttcaaactagataaggaccatgtataagaactatatacttccaatataatatagtataagctttaagatagtatctctcgatctaccgttccacgtgactagtccaaggattttttttaagccaatgaa

SEQ ID NO: 40 - DNA, 1170 bp, Artificial sequence
  Synthesized ispA
aacgagttcgaacgcggcgtgcagctggcacgtcaggggcaggccaaattacaacaagccgaacagcgcgtacaaattctgctgtctgacaatgaagacgcctctctaaccccttttacaccggacaatgagtaatggactttccgcagcaactcgaagcctgcgttaagcaggccaaccaggcgctgagccgttttatcgccccactgccctttcagaacactcccgtggtcgaaaccatgcagtatggcgcattattaggtggtaagcgcctgcgacctttcctggtttatgccaccggtcatatgttcggcgttagcacaaacacgctggacgcacccgctgccgccgttgagtgtatccacgcttactcattaattcatgatgatttaccggcaatggatgatgacgatctgcgtcgcggtttgccaacctgccatgtgaagtttggcgaagcaaacgcgattctcgctggcgacgctttacaaacgctggcgttctcgattttaagcgatgccgatatgccggaagtgtcggaccgcgacagaatttcgatgatttctgaactggcgagcgccagtggtattgccggaatgtgcggtggtcaggcattagatttagacgcggaaggcaaacacgtacctctggacgcgcttgagcgtattcatcgtcataaaaccggcgcattgattcgcgccgccgttcgccttggtgcattaagcgccggagataaaggacgtcgtgctctgccggtactcgacaagtatgcagagagcatcggccttgccttccaggttcaggatgacatcctggatgtggtgggagatactgcaacgttgggaaaacgccagggtgccgaccagcaacttggtaaaagtacctaccctgcacttctgggtcttgagcaagcccggaagaaagcccgggatctgatcgacgatgcccgtcagtcgctgaaacaactggctgaacagtcactcgatacctcggcactggaagcgctagcggactacatcatccagcgtaataaataaacaataagtattaataggcccctgatgagttttgatattgccaaatacccgaccctggcactggtcgactccacccaggagttacgactgttgccgaaagagagtttaccgaaactctgcgacgaactgcgccgct

SEQ ID NO: 41 - DNA, 1931 bp, Artificial sequence
  Synthesized afs1
ttcttgtatcccaaacatctcgagcttcttgtacaccaaattaggtattcactatggaattcagagttcacttgcaagctgataatgagcagaaaatttttcaaaaccagatgaaacccgaacctgaagcctcttacttgattaatcaaagacggtctgcaaattacaagccaaatatttggaagaacgatttcctagatcaatctcttatcagcaaatacgatggagatgagtatcggaagctgtctgagaagttaatagaagaagttaagatttatatatctgctgaaacaatggatttagtagctaagttggagctcattgacagcgtccgaaaactaggcctcgcgaacctcttcgaaaaggaaatcaaggaagccctagacagcattgcagctatcgaaagcgacaatctcggcacaagagacgatctctatggtactgcattacacttcaagatcctcaggcagcatggctataaagtttcacaagatatatttggtagattcatggatgaaaagggcacattagagaaccaccatttcgcgcatttaaaaggaatgctggaacttttcgaggcctcaaacctgggtttcgaaggtgaagatattttagatgaggcgaaagcttccttgacgctagctctcagagatagtggtcatatttgttatccagacagtaacctttccagggacgtagttcattccctggagcttccatcacaccgcagagtgcagtggtttgatgtcaaatggcaaatcaacgcctatgaaaaagacatttgtcgcgtcaacgccacgttactcgaattagcaaagcttaatttcaacgtagttcaggcccaactccaaaaaaacttaagggaagcatccaggtggtgggcaaacctgggcatcgcagacaacttgaaatttgcaagagatagactggttgaatgtttcgcatgtgctgtgggagtagcattcgagcctgagcactcatcttttagaatatgtcttaccaaagtcatcaacttagtactgatcatagacgacgtctatgatatttatggctcagaggaagagctaaagcacttcaccaatgctgttgataggtgggattctagggaaactgagcagcttccagagtgtatgaagatgtgtttccaagtactctacaacactacttgtgaaattgctcgtgaaattgaggaggagaatggttggaaccaagtattacctcaattgaccaaagtgtgggcagatttttgtaaagcattattggtggaggcagagtggtataataagagccatataccaacccttgaagagtacctaagaaacggatgcatttcatcatcagtttcagtgcttttggttcactcgtttttctctataactcatgagggaaccaaagagatggctgattttcttcacaagaatgaagatcttttgtataatatctctctcatcgttcgcctcaacaatgatttgggaacttccgcggctgaacaagagagaggggattctccttcatcaatcgtatgttacatgagagaagtgaatgcctctgaagaaacagctaggaagaacattaagggcatgatagacaatgcatggaagaaagtaaatggaaaatgcttcacaacaaaccaagtgccttttctgtcatcattcatgaacaatgccacaaacatggcacgtgtggcgcacagcctttacaaagatggagatgggtttggtgaccaagagaaagggcctcggacccacatcctgtctttactattccaacctcttgtaaactagtactcatatagtttgaaataaatagcagcaaaagtttgcggttcagttcgtcatggataaattaatctttacagtttgtaacgttgttgccaaagattatgaataaaaagttgtagtttgtcgtttaaaaaaaaaaaaaaaaaaaaa